(19) United States
(12) Patent Application Publication
(10) Pub. No.: US 2026/0201061 A1
(43) Pub. Date: Jul. 16, 2026

(54) COMBINATION FOR TREATMENT AND PREVENTION OF CANCER

(71) Applicant: Hummingbird Bioscience Pte. Ltd., Singapore (SG)

(72) Inventors: Piers Ingram, Singapore (SG); Jerome Boyd-kirup, Singapore (SG); Dipti Thakkar, Singapore (SG); Eric Rowinsky, Houston, TX (US); Weiyi Toy, Singapore (SG)

(21) Appl. No.: 19/136,255

(22) PCT Filed: Dec. 6, 2023
(86) PCT No.: PCT/EP2023/084512

§ 371 Date: Jun. 5, 2025

Related U.S. Application Data
(60) Provisional application No. 63/430,942, filed on Dec. 7, 2022.
(60) Provisional application No. 63/459,373, filed on Apr. 14, 2023.

(51) Int. Cl. C07K 16/32; A61K 31/337; A61K 39/00; A61P 35/00; C07K 16/28

(52) U.S. Cl. CPC C07K 16/32; A61K 31/337; A61P 35/00; C07K 16/2863; A61K 2039/507; C07K 2317/565

(57) ABSTRACT

The present disclosure provides methods for the treatment or prevention of cancers, comprising administration of antigen-binding molecules that bind to HER3 and antigen-binding molecules that bind to EGFR. Also provided are pharmaceutical combinations comprising said molecules, and therapeutic and prophylactic methods of using said pharmaceutical combinations.

This application claims priority from U.S. 63/430,942 filed 7 Dec. 2022, the contents and elements of which are herein incorporated by reference for all purposes.

### TECHNICAL FIELD

The present disclosure relates to medical treatment and prophylaxis, particularly of cancers.

### BACKGROUND

Forster et al., Eur J Cancer. (2019) 123:36-47 discloses the treatment of recurrent or metastatic squamous cell carcinoma of the head and neck (SCCHN) using the combination of anti-HER3 antibody patritumab and the anti-EGFR antibody cetuximab. Patritumab (also known as U-1287 and AMG-888) has been shown to inhibit HER3-mediated signaling by blocking binding of heregulin (HRG) to HER3 (see e.g. Shimizu et al. Cancer Chemother Pharmacol. (2017) 79(3):489-495).

Cleary et al., Investigational New Drugs (2017) 35:68-78 discloses administration of the combination of anti-HER3 antibody seribantumab and cetuximab for the treatment of EGFR-dependent cancers. Like patritumab, seribantumab (also known as MM-121) has been shown to inhibit HER3-mediated signaling by blocking binding of heregulin (HRG) to HER3 (Schoeberl et al., Sci. Signal. (2009) 2(77): ra31).

Meulendijks et al., Clin Cancer Res. (2017) 23(18):5406-5415 discloses administration of the combination of anti-HER3 antibody lumretuzumab and cetuximab for the treatment of advanced HER3-positive carcinomas. Only modest clinical activity was observed. Lumretuzumab (also known as RG7116 and RO-5479599) recognizes an epitope in subdomain I of the HER3 extracellular domain (see e.g. Mirschberger et al. Cancer Research (2013) 73(16) 5183-5194).

Bauman et al., Cancers (Basel) (2022) 14(10): 2355 reports the results of a phase II trial of anti-HER3 antibody CDX-3379 in combination with cetuximab, for the treatment of recurrent/metastatic, HPV-negative, cetuximab-resistant head and neck squamous cell carcinoma (HNSCC). The objective response rate was modest. CDX-3379 (also known as KTN3379) binds to HER3 through interaction with amino acid residues in subdomain III (corresponding to the following positions of SEQ ID NO:1: Gly476, Pro477, Arg481, Gly452, Arg475, Ser450, Gly420, Ala451, Gly419, Arg421, Thr394, Leu423, Arg426, Gly427, Lys356, Leu358, Leu358, Lys356, Ala330, Lys329 and Gly337), and Met310, Glu311 and Pro328 of subdomain II (see Lee et al., Proc Natl Acad Sci USA. 2015 Oct. 27; 112 (43): 13225).

Garner et al., Cancer Res. (2013) 73(19): 6024-6035 discloses the treatment of HNSCC using the combination of anti-HER3 antibody LJM-716 and cetuximab. LJM-716 binds to an epitope on subdomains II and IV of the HER3 extracellular domain, locking HER3 in the inactive conformation (Garner et al., Cancer Res (2013) 73:6024-6035).

Papadopoulos et al., Journal of Clinical Oncology (2014) 32(15_suppl): 2516-2516 discloses administration of the combination of anti-HER3 antibody REGN1400 and cetuximab for the treatment of advanced non-small cell lung cancer (NSCLC), colorectal cancer (CRC), or SCCHN. REGN1400 also inhibits binding of ligand to HER3 (see Zhang et al., Mol Cancer Ther (2014) 13:1345-1355).

Kim et al., Annals of Oncology (2020) 31(suppl_4): S599-S628 disclose the treatment of recurrent or metastatic HNSCC using the combination of anti-HER3 antibody ISU104 and cetuximab. ISU104 (also known as barecetamab) mainly binds to domain III of HER3, weakly interacts with domain I, and displays dose-dependent inhibition of binding of HRG to HER3 (see Kim et al., Cancer Research (2018) 78 (13 Supplement): 830-830).

### SUMMARY

In a first aspect, the present disclosure provides an antigen-binding molecule that binds to HER3 for use in a method of treating or preventing a cancer, wherein the method comprises administering an antigen-binding molecule that binds to EGFR, and wherein the antigen-binding molecule that binds to HER3 binds to the region of HER3 shown in SEQ ID NO:77. In some embodiments, the method of treating or preventing a cancer further comprises administering docetaxel.

The present disclosure also provides the use of an antigen-binding molecule that binds to HER3 in the manufacture of a medicament for use in a method of treating or preventing a cancer, wherein the method comprises administering an antigen-binding molecule that binds to EGFR, and wherein the antigen-binding molecule that binds to HER3 binds to the region of HER3 shown in SEQ ID NO:77. In some embodiments, the method of treating or preventing a cancer further comprises administering docetaxel.

The present disclosure also provides a method of treating or preventing a cancer, comprising administering to a subject a therapeutically- or prophylactically-effective amount of (i) an antigen-binding molecule that binds to HER3 and (ii) an antigen-binding molecule that binds to EGFR; wherein the antigen-binding molecule that binds to HER3 binds to the region of HER3 shown in SEQ ID NO:77. In some embodiments, the method of treating or preventing a cancer further comprises administering docetaxel.

The present disclosure also provides a pharmaceutical combination, comprising an antigen-binding molecule that binds to HER3 and an antigen-binding molecule that binds to EGFR; wherein the antigen-binding molecule that binds to HER3 binds to the region of HER3 shown in SEQ ID NO:77.

The present disclosure also provides a pharmaceutical combination according to the present disclosure, for use in a method of treating or preventing a cancer. In some embodiments, the method of treating or preventing a cancer further comprises administering docetaxel.

The present disclosure also provides the use of a pharmaceutical combination according to the present disclosure in the manufacture of a medicament for treating or preventing a cancer. In some embodiments, the method of treating or preventing a cancer further comprises administering docetaxel.

The present disclosure also provides a method of treating or preventing a cancer, comprising administering to a subject a therapeutically- or prophylactically-effective amount of a pharmaceutical combination according to the present disclosure. In some embodiments, the method of treating or preventing a cancer further comprises administering docetaxel.

In some embodiments, the antigen-binding molecule that binds to HER3 comprises:
(i) a heavy chain variable (VH) region incorporating the following CDRs:
   HC-CDR1 having the amino acid sequence of SEQ ID NO:40
   HC-CDR2 having the amino acid sequence of SEQ ID NO:43
   HC-CDR3 having the amino acid sequence of SEQ ID NO:48; and
(ii) a light chain variable (VL) region incorporating the following CDRs:
   LC-CDR1 having the amino acid sequence of SEQ ID NO:66
   LC-CDR2 having the amino acid sequence of SEQ ID NO:69
   LC-CDR3 having the amino acid sequence of SEQ ID NO:74.

In some embodiments, the antigen-binding molecule that binds to HER3 comprises:
(i) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45; and
(ii) a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70.

In some embodiments, the antigen-binding molecule that binds to HER3 comprises:
a VH region comprising an amino acid sequence having at least 70% sequence identity to the amino acid sequence of SEQ ID NO:33; and
a VL region comprising an amino acid sequence having at least 70% sequence identity to the amino acid sequence of SEQ ID NO:58.

In some embodiments, the antigen-binding molecule that binds to HER3 comprises:
a polypeptide comprising, or consisting of, an amino acid sequence having at least 70% sequence identity to the amino acid sequence of SEQ ID NO:75; and
a polypeptide comprising, or consisting of, an amino acid sequence having at least 70% sequence identity to the amino acid sequence of SEQ ID NO:76.

In some embodiments, the antigen-binding molecule that binds to EGFR comprises:
(i) a heavy chain variable (VH) region incorporating the following CDRs:
   HC-CDR1 having the amino acid sequence of SEQ ID NO:92
   HC-CDR2 having the amino acid sequence of SEQ ID NO:93
   HC-CDR3 having the amino acid sequence of SEQ ID NO:94; and
(ii) a light chain variable (VL) region incorporating the following CDRs:
   LC-CDR1 having the amino acid sequence of SEQ ID NO:96
   LC-CDR2 having the amino acid sequence of SEQ ID NO:97
   LC-CDR3 having the amino acid sequence of SEQ ID NO:98.

In some embodiments, the antigen-binding molecule that binds to EGFR comprises:
a VH region comprising an amino acid sequence having at least 70% sequence identity to the amino acid sequence of SEQ ID NO:91; and
a VL region comprising an amino acid sequence having at least 70% sequence identity to the amino acid sequence of SEQ ID NO:95.

In some embodiments, the antigen-binding molecule that binds to EGFR comprises:
a polypeptide comprising, or consisting of, an amino acid sequence having at least 70% sequence identity to the amino acid sequence of SEQ ID NO:99; and
a polypeptide comprising, or consisting of, an amino acid sequence having at least 70% sequence identity to the amino acid sequence of SEQ ID NO:100.

In some embodiments, the cancer is selected from: a cancer comprising cells expressing/overexpressing an EGFR family member, a cancer comprising cells expressing/overexpressing HER3, a cancer comprising cells expressing/overexpressing EGFR, a cancer comprising cells expressing/overexpressing HER3 and EGFR, a cancer comprising cells having a mutation resulting in increased expression of a ligand for HER3, a cancer comprising cells having a mutation resulting in increased expression of a ligand for EGFR, a cancer comprising cells having an NRG gene fusion, a solid tumor, a hematological cancer, a squamous cell cancer, an EGFR-amplified squamous cell carcinoma, breast cancer, breast carcinoma, breast invasive carcinoma, ductal carcinoma, metastatic breast cancer, triple-negative breast cancer, HER2-positive breast cancer, gastric cancer, gastric carcinoma, gastric adenocarcinoma, gastrointestinal adenocarcinoma, colorectal cancer, metastatic colorectal cancer, colon cancer, colorectal carcinoma, colorectal adenocarcinoma, colon adenocarcinoma, head and neck cancer, head and neck squamous cell carcinoma, lung cancer, non-small cell lung cancer, lung adenocarcinoma, invasive mucinous lung adenocarcinoma, lung squamous cell carcinoma, ovarian cancer, ovarian carcinoma, ovarian serous adenocarcinoma, ovarian serous cystadenocarcinoma, fallopian tube cancer, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, oral cavity cancer, oropharyngeal cancer, esophageal cancer, esophageal squamous cell carcinoma, esophageal adenocarcinoma, liver cancer, hepatocellular carcinoma, cholangiocarcinoma, gallbladder cancer, uterine cancer, endometrial cancer, uterine corpus endometrial carcinoma, uterine carcinosarcoma, thyroid cancer, thyroid cancer, thyroid carcinoma, pheochromocytoma, paraganglioma, bladder cancer, bladder urothelial carcinoma, prostate cancer, prostate adenocarcinoma, retinoblastoma, sarcoma, soft tissue sarcoma, peritoneal cancer, thymoma, neuroendocrine tumor, neuroendocrine tumor of the nasopharynx, squamous cell carcinoma of the skin, astrocytoma, low grade astrocytoma, high grade astrocytoma, anaplastic astrocytoma and glioblastoma multiforme.

In some embodiments, the cancer is selected from: a cancer comprising cells expressing/overexpressing HER3, a cancer comprising cells expressing/overexpressing EGFR, a cancer comprising cells expressing/overexpressing HER3 and EGFR, a squamous cell cancer, an EGFR-amplified squamous cell carcinoma, esophageal cancer, esophageal squamous cell carcinoma, head and neck cancer, head and neck squamous cell carcinoma, colorectal cancer, metastatic colorectal cancer, colon adenocarcinoma, pancreatic cancer, pancreatic adenocarcinoma, pancreatic ductal adenocarcinoma, lung cancer and lung squamous cell carcinoma.

### DESCRIPTION

The present disclosure relates to a combination treatment for cancer, comprising an antigen-binding molecule that binds to HER3, and an antigen-binding molecule that binds to EGFR. In preferred embodiments, the antigen-binding molecule that binds to HER3 is 10D1F, and the antigen-binding molecule that binds to EGFR is cetuximab.

10D1F binds to an epitope of HER3 which is different to the epitopes of anti-HER3 antibodies employed in combination treatments disclosed in the prior art comprising an anti-HER3 antibody and cetuximab. More specifically, 10D1F binds the dimerization interface of HER3, while other anti-HER3 antibodies bind on various sites of HER3 extracellular domain (e.g. the ligand binding domain, domains II/IV). As 10D1F inhibits HER3 dimerization with its receptor partners and blocks both ligand-dependent and ligand-independent activation of HER3, it provides more complete shutdown of HER3-mediated signaling.

In the experimental examples of the present application, the inventors demonstrate that the combination of 10D1F and cetuximab is provided with unexpected and advantageous properties over combination treatments comprising anti-HER3 antibody and cetuximab disclosed in the prior art. The combination of 10D1F and cetuximab is also provided with unexpected and advantageous effects as an intervention for the treatment/prevention of various different cancers, as compared to either constituent component of the combination employed as a monotherapy.

### HER3

HER3 (also known e.g. as ERBB3, LCCS2, MDA-BF-1) is the protein identified by UniProt P21860.

The structure and function of HER3 is described e.g. in Cho and Leahy Science (2002) 297 (5585): 1330-1333, Singer et al., Journal of Biological Chemistry (2001) 276, 44266-44274, Roskoski et al., Pharmacol. Res. (2014) 79:34-74, Bazley and Gullick Endocrine-Related Cancer (2005) S17-S27 and Mujoo et al., Oncotarget (2014) 5 (21): 10222-10236, each of which are hereby incorporated by reference in their entirety. HER3 is a single-pass transmembrane ErbB receptor tyrosine kinase having an N-terminal extracellular region (SEQ ID NO:9) comprising two leucine-rich subdomains (domains I and III, shown in SEQ ID NOs: 15 and 17, respectively) and two cysteine-rich subdomains (domains II and IV, shown in SEQ ID NOs: 16 and 18, respectively). Domain II comprises a β hairpin dimerization loop (SEQ ID NO:19) which is involved in intermolecular interactions with other HER receptor molecules. The extracellular region is linked via a transmembrane region (SEQ ID NO:10) to a cytoplasmic region (SEQ ID NO:11).

The cytoplasmic region comprises a juxtamembrane segment (SEQ ID NO:12), a protein kinase domain (SEQ ID NO:13), and a C-terminal segment (SEQ ID NO:14).

In this specification ‘HER3’ refers to HER3 from any species and includes HER3 isoforms, fragments, variants (including mutants) or homologues from any species.

As used herein, a ‘fragment’, ‘variant’ or ‘homologue’ of a protein may optionally be characterized as having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference protein (e.g. a reference isoform). In some embodiments, fragments, variants, isoforms and homologues of a reference protein may be characterized by ability to perform a function performed by the reference protein.

A ‘fragment’ generally refers to a fraction of the reference protein. A ‘variant’ generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An ‘isoform’ generally refers to a variant of the reference protein expressed by the same species as the species of the reference protein (e.g. human HER3 isoforms 1 to 5 are all isoforms of one another). A ‘homologue’ generally refers to a variant of the reference protein produced by a different species as compared to the species of the reference protein. For example, human HER3 isoform 1 (P21860-1, v1; SEQ ID NO:1) and Rhesus macaque HER3 (UniProt: F7HEH3-1, v2; SEQ ID NO:20) are homologues of one another. Homologues include orthologues.

A ‘fragment’ of a reference protein may be of any length (by number of amino acids), although may optionally be at least 20% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein.

A fragment of HER3 may have a minimum length of one of 10, 20, 30, 40, 50, 100, 150, 200, 250, 300, 350, 400, 450, 500, 600, 700, 800, 900, 1000, 1100, 1200 amino acids, and may have a maximum length of one of 20, 30, 40, 50, 100, 150, 200, 250, 300, 350, 400, 450, 500, 600, 700, 800, 900, 1000, 1100, 1200, or 1300 amino acids.

In some embodiments, the HER3 is HER3 from a mammal (e.g. a primate (rhesus, cynomolgous, non-human primate or human) and/or a rodent (e.g. rat or murine) HER3). Isoforms, fragments, variants or homologues of HER3 may optionally be characterized as having at least 70%, preferably one of 80%, 85%, 90%, 91%, 92%, 93%, 94%, 95%, 96%, 97%, 98%, 99% or 100% amino acid sequence identity to the amino acid sequence of an immature or mature HER3 isoform from a given species, e.g. human.

Isoforms, fragments, variants or homologues may optionally be functional isoforms, fragments, variants or homologues, e.g. having a functional property/activity of the reference HER3 (e.g. human HER3 isoform 1), as determined by analysis by a suitable assay for the functional property/activity. For example, an isoform, fragment, variant or homologue of HER3 may display association with one or more of: HER2, NRG1 (type I, II, III, IV, V or VI) or NRG2 (α or β).

In some embodiments, the HER3 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs: 1 to 8.

In some embodiments, a fragment of HER3 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs: 9 to 19, e.g. one of 9, 16 or 19.

Signaling through HER3 involves receptor heteromultimerization (i.e. with other ErBB receptors, e.g. HER2, EGFR) and consequent autophosphorylation by the protein kinase domain of tyrosine residues of the cytoplasmic region. HER3 lacks kinase activity and does not form stable homodimers. Therefore, HER3 must be transphosphorylated by binding to a kinase-active heterodimer partner (e.g. EGFR or HER2) for signal transduction to take place (Berger M B et al., FEBS Lett 2004; 569:332-6; Kim H H et al., Biochem J 1998; 334:189-95).

Multimerization (e.g. dimerization) of HER receptor family members is required for activating cell growth signaling pathways, and HER3 can dimerize with other HER family members in both a ligand-dependent and ligand-independent manner. The HER3 extracellular domain (ECD) exists in a reversible equilibrium between a ‘closed’ inactive conformation and an ‘open’ active conformation, in which the dimerization arm within domain II is exposed to allow dimerization along the domain II dimerization interface, and in particular through the cysteine-rich CR1 region (Carraway, K. L., et al., Nature, 1997. 387(6632): 512-6; Riese, D. J., et al., Mol Cell Biol, 1995. 15(10): 5770-6; Harari, D., et al., Oncogene, 1999. 18(17): 2681-9; Zhang, D., et al., Proc Natl Acad Sci USA, 1997. 94(18): 9562-7; Meyer et al., Nature, 1995. 378(6555):386-90; Jura, N., et al., Proc Natl Acad Sci USA, 2009. 106(51): 21608-13; Fornaro, L., et al., Nat Rev Gastroenterol Hepatol, 2011. 8(7):369-83; Mota et al., Oncotarget (2015) 5:89284-306). HER3 is ‘activated’ when the equilibrium is shifted in favor of the open conformation, increasing the probability of forming active heterodimers. The conventional model for activation is ligand-dependent, that is, the equilibrium shifts when HER3 in the open conformation is stabilized by binding of its ligand such as a neuregulin (NRG), e.g. NRG1 (also known as heregulin, HRG) or NRG2. In addition, the presence of any dimerization partner at a sufficient concentration will shift the equilibrium in favor of the open conformation, as they bind to and stabilize HER3 transiently in an open conformation. This is known as ligand-independent activation (Jura, N., et al., Proc Natl Acad Sci USA, 2009. 106(51): 21608-13; Fornaro, L., et al., Nat Rev Gastroenterol Hepatol, 2011. 8(7): p. 369-83; Mota et al., Oncotarget (2015) 5:89284-306).

Herein, ‘HER3-mediated signaling’ refers to signaling mediated by HER3 and/or multimeric ErbB family member receptor complexes comprising HER3. ‘Signaling’ refers to signal transduction and other cellular processes governing cellular activity. HER3-mediated signaling may be mediated by HER3 receptor-containing complexes, e.g. by heteromultimeric complexes comprising HER3 and other HER receptors (e.g. HER2, EGFR). HER3-mediated signaling may be ligand-dependent, e.g. triggered by binding of NRG (e.g. NRG1, NRG2), or may be ligand-independent.

HER3-mediated signaling progresses intracellularly through the MAPK/ERK and PI3K/AKT/mTOR pathways to promote cell survival and proliferation. HER3-mediated signaling is described e.g. in Gala and Chandarlapaty, Clin Cancer Res. (2014) 20(6): 1410-1416, Mishra et al., Oncol Rev. (2018) 12(1): 355, Baselga et al., Nat Rev Cancer (2009) 9:463-75, Yarden et al. Nat Rev Mol Cell Biol (2001) 2:35052073, Mota et al., Oncotarget (2015) 5:89284-306 and Haikala and Jänne, Clin. Cancer Res. (2021) 27:3528-39, all of which are hereby incorporated by reference in their entirety.

Phosphorylated tyrosine residues in the protein kinase domains of HER3-containing receptor complexes recruit adaptor/effector protein GRB2, via interaction with its SH2 domain. Upon ligand stimulation, the activated receptor (EGFR/HER2) undergoes autophosphorylation and provides phospho-tyrosine residues for recruiting GRB2. GRB2 binds via its SH3 domains to the guanine nucleotide exchange factor SOS. Activated SOS in GRB2-SOS complexes promotes removal of GDP from, and thereby activation of, Ras family GTPases such as H-Ras, N-Ras and K-Ras. Activated Ras GTPases in turn activate RAF kinases such as A-Raf, B-Raf and C-Raf. RAF kinases in turn phosphorylate and activate MEK1 and MEK2, which then phosphorylate and activate MAPKs (also known as ERKs). Activated MAPKs are able to directly regulate the activity of transcription factors such as c-Myc. Activated MAPKs also upregulate translation of mRNA into protein via phosphorylation of RSK, and consequent phosphorylation and activation of 40S ribosomal protein S6. Activated MAPKs also phosphorylate and activate MNK, which in turn phosphorylates and activates the transcription factor CREB.

Phosphorylated tyrosine residues in the protein kinase domain of HER3 also recruit the p85 subunit of PI3K, through its SH2 domain. Association of p85 causes allosteric activation of the lipid kinase p100a subunit of PI3K. Activated PI3K results in conversion of PIP2 to PIP3, which recruits AKT to be phosphorylated and activated by mTORC2 and PDK1. Phosphorylated AKT has a number of activities, including activating CREB and mTOR. PTEN antagonizes signaling through the PI3K/AKT/mTOR pathway by dephosphorylating PIP3 to PIP2, and PP2A inhibits the PI3K/AKT/mTOR pathway by dephosphorylating AKT.

Oncogenic Src homology region 2 protein tyrosine phosphatase 2 (SHP2) promotes tumor progression and serves as a pivotal hub to connect multiple oncogenic signaling pathways, such as PI3K/AKT, Ras/Raf/MAPK (Dong et al., Front. Cell Dev. Biol., 11 Mar. 2021). GAB2 binds to GRB2 and becomes phosphorylated at multiple tyrosine residues, capable of binding to the SH2 domains of SHP2 and p85 (Adams et al., Mol Cancer Res. 2012 October; 10(10):1265-70; (Liu et al., Proc. Natl. Acad. Sci. U.S.A. (2016) 113, 984-989). The interactions induce conformation changes, relieving the auto-inhibition of the SHP2 catalytic site (Neel et al., Trends Biochem Sci. 2003 June; 28(6):284-93) and relieving the inhibition of p85 on the p110 catalytic subunit of PI3K (Cuevas et al., J Biol Chem. 2001 Jul. 20; 276(29):27455-6), respectively. SHP2 has been shown to activate RAS by direct dephosphorylation of RAS (Bunda et al., Nat Commun. 2015 Nov. 30; 6:8859), inhibition of RASGAP (RAS GTPase activating protein) (Neel et al., Trends Biochem Sci. 2003 June; 28(6):284-93) and SPRY (Hanafusa et al., J Biol Chem. 2004 May 28; 279(22):22992-5). SHP2 overexpression has been shown to enhance tumor invasion by activating the PI3K/AKT axis (Hu et al., Onco Targets Ther. (2017) 10, 3881-3891) while SHP2 knockdown inhibits cell migration and the tumor-promoting effect of SHP2 is partially related to AKT signaling (Cao et al., Pathol. Res. Pract. (2019) 215:152621).

STAT3 and 5 proteins are transcription factors that enhance the expression of p85a, p110a and AKT1 and thereby augment signaling through the PI3K/AKT signal transduction cascade (Radler et al., Mol Cell Endocrinol. 2017 Aug. 15; 451:31-39). Upon activation by JAK2, phosphorylated STAT5 binds to the SH2 domain of the p85a regulatory subunit of PI3K in a PRL signaling-dependent manner suggesting that STAT5 may also directly participate in the signaling of PI3K complexes. Another kinase that phosphorylates EGFR is the cytokine-regulated tyrosine kinase Jak2, thus allowing MAPK activation even by a kinase-defective mutant of EGFR (Mishra et al., Oncol Rev. (2018) 12 (1): 355, Baselga et al., Nat Rev Cancer (2009) 9:463-75). The collective observations in genetic models overexpressing or lacking active STAT5 and AKT or expressing mutant PTEN supports the notion that STAT5 functions as a survival factor during normal mammary gland development and as an oncogene during mammary carcinogenesis are mediated by the PI3K/AKT pathway (Radler et al., Mol Cell Endocrinol. 2017 Aug. 15; 451:31-39).

### EGFR

EGFR (also known as e.g. ERBB1, HER1) is the protein identified by UniProt P00533.

The structure and function of EGFR is described e.g. in Sabbah et al., Curr Top Med Chem. (2020) 20(10):815-834 and Sigismund et al., Mol Oncol. (2018) 12(1):3-20, both of which are hereby incorporated by reference in their entirety. EGFR is a single-pass transmembrane ErbB receptor tyrosine kinase having an N-terminal extracellular region (SEQ ID NO:88), which is linked via a transmembrane region (SEQ ID NO:89) to a cytoplasmic region (SEQ ID NO:90).

In this specification ‘EGFR’ refers to EGFR from any species and includes EGFR isoforms, fragments, variants (including mutants) or homologues from any species.

A fragment of EGFR may have a minimum length of one of 10, 20, 30, 40, 50, 100, 150, 200, 250, 300, 350, 400, 450, 500, 600, 700, 800, 900, 1000, 1100, 1200 amino acids, and may have a maximum length of one of 20, 30, 40, 50, 100, 150, 200, 250, 300, 350, 400, 450, 500, 600, 700, 800, 900, 1000, 1100, or 1200 amino acids.

In some embodiments, the EGFR is EGFR from a mammal (e.g. a primate (rhesus, cynomolgous, non-human primate or human) and/or a rodent (e.g. rat or murine) EGFR). Isoforms, fragments, variants or homologues of EGFR may optionally be characterized as having at least 70%, preferably one of 80%, 85%, 90%, 91%, 92%, 93%, 94%, 95%, 96%, 97%, 98%, 99% or 100% amino acid sequence identity to the amino acid sequence of an immature or mature EGFR isoform from a given species, e.g. human.

Isoforms, fragments, variants or homologues may optionally be functional isoforms, fragments, variants or homologues, e.g. having a functional property/activity of the reference EGFR (e.g. human EGFR isoform 1), as determined by analysis by a suitable assay for the functional property/activity. For example, an isoform, fragment, variant or homologue of EGFR may display association with one or more of: HER3, HER2, EGF, TGFα and amphiregulin.

In some embodiments, the EGFR comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs: 80 to 87.

In some embodiments, a fragment of EGFR comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs: 88, 89 or 90.

EGFR-mediated signaling is described e.g. in Sigismund et al., Mol Oncol. (2018) 12 (1): 3-20 and Kovacs et al., Annu Rev Biochem. (2015) 84:739-764, both of which are hereby incorporated in their entirety. Canonical EGFR signaling is critical for various cellular functions including survival, proliferation, differentiation, and motility. In the absence of ligand, EGFR is mostly present in the plasma membrane in an auto-inhibited, dimerization-incompetent state. Ligand binding induces receptor dimerization and aggregation of catalytic regions, resulting in trans-autophosphorylation of key tyrosine residues in the cytoplasmic domain and triggering the intracellular signaling cascade. There are seven known EGFR ligands, which differ in the kind and strength of downstream signaling they elicit. EGFR can also heterodimerize with HER2, HER3 and HER4. Signaling from heterodimers is predicted to be more oncogenic than signaling from EGFR homodimers.

EGFR activation triggers multiple intracellular signaling pathways, including the Ras/Raf/MAPK pathway, the PI3K/AKT pathway, and the phospholipase C (PLC)/protein kinase C (PKC) signaling cascade.

### Antigen-Binding Molecules

The present disclosure relates to the therapeutic and prophylactic use of antigen-binding molecules that bind to HER3, and of antigen-binding molecules that bind to EGFR.

An ‘antigen-binding molecule’ refers to a molecule that binds to a given target antigen. Antigen-binding molecules include antibodies (i.e. immunoglobulins (Igs)) and antigen-binding fragments thereof. As used herein, ‘antibodies’ include monoclonal antibodies, polyclonal antibodies, monospecific and multispecific (e.g., bispecific, trispecific, etc.) antibodies, and antibody-derived antigen-binding molecules such as scFv, scFab, diabodies, triabodies, scFv-Fc, minibodies, single domain antibodies (e.g. VhH, etc.). Antigen-binding fragments of antibodies include e.g. Fv, Fab, F(ab′)₂ and F(ab′) fragments. In some embodiments, an antigen-binding molecule may be an antibody or an antigen-binding fragment thereof.

Antigen-binding molecules according to the present disclosure also include antibody-derived molecules, e.g. molecules comprising an antigen-binding region/domain derived from an antibody. Antibody-derived antigen-binding molecules may comprise an antigen-binding region/domain that comprises, or consists of, the antigen-binding region of an antibody (e.g. an antigen-binding fragment of an antibody). In some embodiments, the antigen-binding region/domain of an antibody-derived antigen-binding molecule may be or comprise the Fv (e.g. provided as an scFv) or the Fab region of an antibody, or the whole antibody. For example, antigen-binding molecules according to the present disclosure include antibody-drug conjugates (ADCs) comprising a (cytotoxic) drug moiety (e.g. as described hereinbelow). Antigen-binding molecules according to the present disclosure also include multispecific antigen-binding molecules such as immune cell engager molecules comprising a domain for recruiting (effector) immune cells (reviewed e.g. in Goebeler and Bargou, Nat. Rev. Clin. Oncol. (2020) 17:418-434 and Ellerman, Methods (2019) 154:102-117, both of which are hereby incorporated by reference in their entirety), including BiTEs, BiKEs and TriKEs. Antigen-binding molecules according to the present disclosure also include chimeric anti-gen receptors (CARs), which are recombinant receptors providing both antigen-binding and T cell activating functions (CAR structure, function and engineering is reviewed e.g. in Dotti et al., Immunol Rev (2014) 257(1) and Jayaraman et al., EBioMedicine (2020) 58:102931, both of which are hereby incorporated by reference in their entirety).

The antigen-binding molecules of the present disclosure comprise a moiety or moieties capable of binding to a target antigen(s). In some embodiments, the moiety capable of binding to a target antigen comprises an antibody heavy chain variable region (VH) and an antibody light chain variable region (VL) of an antibody capable of specific binding to the target antigen. In some embodiments, the moiety capable of binding to a target antigen comprises or consists of an aptamer capable of binding to the target antigen, e.g. a nucleic acid aptamer (reviewed, for example, in Zhou and Rossi Nat Rev Drug Discov. 2017 16(3):181-202). In some embodiments, the moiety capable of binding to a target antigen comprises or consists of an antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

As used herein, a ‘peptide’ refers to a chain of two or more amino acid monomers linked by peptide bonds. A peptide typically has a length in the region of about 2 to 50 amino acids. A ‘polypeptide’ is a polymer chain of two or more peptides. Polypeptides typically have a length greater than about 50 amino acids.

The antigen-binding molecules of the present disclosure generally comprise an antigen-binding domain comprising a VH and a VL of an antibody capable of specific binding to the target antigen. The antigen-binding domain formed by a VH and a VL may also be referred to herein as an Fv region.

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. In some embodiments, the polypeptides form part of a larger polypeptide comprising the polypeptides (e.g. in the case of scFv comprising VH and VL, or in the case of scFab comprising VH—CH1 and VL-CL).

An antigen-binding molecule may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The antigen-binding molecules of the present disclosure may be designed and prepared using the sequences of monoclonal antibodies (mAbs) capable of binding to a given target antigen (e.g. HER3 or EGFR). Antigen-binding regions of antibodies, such as single chain variable fragment (scFv), Fab and F(ab′)₂ fragments may also be used/provided. An ‘antigen-binding region’ is any fragment of an antibody that binds to the target for which the given antibody is specific.

Antibodies generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody that binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015) 43 (Database issue):D413-22), which uses the IMGT V-DOMAIN numbering rules as described in Lefranc et al., Dev. Comp. Immunol. (2003) 27:55-77. In preferred embodiments, the CDRs and FRs of antigen-binding molecules referred to herein are defined according to the IMGT information system.

The VH and VL region of an antigen-binding region of an antibody together constitute the Fv region. In some embodiments, an antigen-binding molecule according to the present disclosure comprises, or consists of, an Fv region that binds to HER3. In some embodiments, an antigen-binding molecule according to the present disclosure comprises, or consists of, an Fv region that binds to EGFR. In some embodiments, the VH and VL regions of the Fv are provided as single polypeptide joined by a linker sequence, i.e. a single chain Fv (scFv).

The VL and light chain constant (CL) region, and the VH region and heavy chain constant 1 (CH1) region of an antigen-binding region of an antibody together constitute the Fab region. In some embodiments, the antigen-binding molecule comprises a Fab region comprising a VH, a CH1, a VL and a CL (e.g. Cκ or Cλ). In some embodiments, the Fab region comprises a polypeptide comprising a VH and a CH1 (e.g. a VH—CH1 fusion polypeptide), and a polypeptide comprising a VL and a CL (e.g. a VL-CL fusion polypeptide). In some embodiments, the Fab region comprises a polypeptide comprising a VH and a CL (e.g. a VH-CL fusion polypeptide) and a polypeptide comprising a VL and a CH (e.g. a VL-CH1 fusion polypeptide); that is, in some embodiments, the Fab region is a CrossFab region. In some embodiments, the VH, CH1, VL and CL regions of the Fab or CrossFab are provided as single polypeptide joined by linker regions, i.e. as a single chain Fab (scFab) or a single chain CrossFab (scCrossFab).

In some embodiments, an antigen-binding molecule described herein comprises, or consists of, a whole antibody which binds to HER3. In some embodiments, an antigen-binding molecule described herein comprises, or consists of, a whole antibody which binds to EGFR. As used herein, ‘whole antibody’ refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are ~150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments, the antigen-binding molecule comprises, or consists of, an IgG (e.g. IgG1, IgG2, IgG3, IgG4), IgA (e.g. IgA1, IgA2), IgD, IgE, or IgM which binds to HER3. In some embodiments, the antigen-binding molecule comprises, or consists of, an IgG (e.g. IgG1, IgG2, IgG3, IgG4), IgA (e.g. IgA1, IgA2), IgD, IgE, or IgM which binds to EGFR.

In some embodiments described herein, one or more amino acids of an amino acid sequence referred to herein (e.g. an amino acid sequence of an antigen-binding molecule, e.g. an amino acid sequence of a CDR or VH/VL region) are substituted with another amino acid. A substitution comprises substitution of an amino acid residue with a non-identical ‘replacement’ amino acid residue. A replacement amino acid residue of a substitution according to the present disclosure may be a naturally-occurring amino acid residue (i.e. encoded by the genetic code) which is non-identical to the amino acid residue at the relevant position of the equivalent, unsubstituted amino acid sequence, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (Ile): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid may be a non-naturally occurring amino acid residue—i.e. an amino acid residue other than those recited in the preceding sentence. Examples of non-naturally occurring amino acid residues include norleucine, ornithine, norvaline, homoserine, aib, and other amino acid residue analogues such as those described in Ellman, et al., Meth. Enzym. 202 (1991) 301-336.

In some embodiments, a substitution may be biochemically conservative. In some embodiments, where an amino acid to be substituted is provided in one of rows 1 to 5 of the table below, the replacement amino acid of the substitution is another, non-identical amino acid provided in the same row:

| Row | Shared property | Amino acids |
|---|---|---|
| 1 | Hydrophobic | Met, Ala, Val, Leu, Ile, Trp, |
| | | Tyr, Phe, Norleucine |
| 2 | Neutral hydrophilic | Cys, Ser, Thr, Asn, Gln |
| 3 | Acidic or negatively-charged | Asp, Glu |
| 4 | Basic or positively-charged | His, Lys, Arg |
| 5 | Orientation influencing | Gly, Pro |

By way of illustration, in some embodiments wherein substitution is of a Met residue, the replacement amino acid may be selected from Ala, Val, Leu, Ile, Trp, Tyr, Phe and Norleucine.

In some embodiments, a replacement amino acid in a substitution may have the same side chain polarity as the amino acid residue it replaces. In some embodiments, a replacement amino acid in a substitution may have the same side chain charge (at pH 7.4) as the amino acid residue it replaces:

| | | Side-chain |
|---|---|---|
| | Side-chain | charge |
| Amino Acid | polarity | (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another, non-identical nonpolar amino acid. In some embodiments, a polar amino acid is substituted with another, non-identical polar amino acid. In some embodiments, an acidic polar amino acid is substituted with another, non-identical acidic polar amino acid. In some embodiments, a basic polar amino acid is substituted with another, non-identical basic polar amino acid. In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally conservative. That is, in some embodiments, the substitution may not affect (or may not substantially affect) one or more functional properties (e.g. target binding) of the antigen-binding molecule comprising the substitution as compared to the equivalent unsubstituted molecule.

Antigen-Binding Molecules that Bind to Her3

The present disclosure provides antigen-binding molecules that bind to HER3.

In some embodiments, the antigen-binding molecule comprises the CDRs of an antigen-binding molecule which is capable of binding to HER3. In some embodiments, the antigen-binding molecule comprises the FRs of an antigen-binding molecule which is capable of binding to HER3. In some embodiments, the antigen-binding molecule comprises the CDRs and the FRs of an antigen-binding molecule which is capable of binding to HER3. That is, in some embodiments the antigen-binding molecule comprises the VH region and the VL region of an antigen-binding molecule which is capable of binding to HER3.

In some embodiments, an antigen-binding molecule which is capable of binding to HER3 according to the present disclosure may be selected from: any embodiment of an antigen-binding molecule described in WO 2019/185878 A1 (which is hereby incorporated by reference in its entirety), 10D1F (described e.g. in WO 2019/185878 A1), seribantumab (also known as MM-121, described e.g. in Schoeberl et al., Sci. Signal. (2009) 2(77): ra31; DrugBank Acc. No. DB11857), elgemtumab (also known as LJM-716, described e.g. in Garner et al., Cancer Res (2013) 73:6024-6035; DrugBank Acc. No. DB15430), patritumab (also known as U-1287 and AMG-888, described e.g. in Shimizu et al. Cancer Chemother Pharmacol. (2017) 79(3): 489-495; DrugBank Acc. No. DB12090), GSK2849330 (described e.g. in Clarke et al., Eur J Cancer. (2014) 50:98-9), lumretuzumab (also known as RG7116 and RO-5479599, described e.g. in Mirschberger et al. Cancer Research (2013) 73(16) 5183-5194; DrugBank Acc. No. DB12683), CDX-3379 (also known as KTN3379, described e.g. in Lee et al., Proc Natl Acad Sci USA. 2015 Oct. 27; 112(43): 13225), AV-203 (also known as CAN-017, described e.g. in Meetze et al., Eur J Cancer 2012; 48:126), barecetamab (also known as ISU104, described e.g. in Kim et al., Cancer Res (2018) 78(13 Suppl):Abstract #830), TK-A3, TK-A4 (described e.g. in Malm et al., MAbs (2016) 8:1195-209), MP-EV20 (described e.g. in Sala et al., Transl. Oncol. (2013) 6:676-84), 1A5-3D4 (described e.g. in Wang et al., Cancer Lett (2016) 380:20-30), 9F7-F11, 16D3-C₁ (described e.g. in Lazrek et al., Neoplasia (2013) 15:335-47), NG33, A5, F4 (described e.g. in Gaborit et al., PNAS USA (2015) 112:839-44), huHER3-8 (described e.g. in Kugel et al., Cancer Res. (2014) 74:4122-32), REGN1400 (described e.g. in Zhang et al., Mol Cancer Ther (2014) 13:1345-1355) and zenocutuzumab (also known as MCLA-128, described e.g. in de Vries Schultink et al., Clin Pharmacokinet. (2020) 59:875-884; DrugBank Acc. No. DB15559). In some embodiments, the antigen-binding molecule is 10D1F.

In some embodiments, the antigen-binding molecule binds to the extracellular region of HER3 (e.g. the region shown in SEQ ID NO:9). In some embodiments, the antigen-binding molecule binds to subdomain II of the extracellular region of HER3 (e.g. the region shown in SEQ ID NO:16).

In some embodiments, the antigen-binding molecule binds to the region of HER3 shown in SEQ ID NO: 77. In some embodiments the antigen-binding molecule contacts one or more amino acid residues of the region of HER3 shown in SEQ ID NO:77. In some embodiments, the antigen-binding molecule binds to the regions of HER3 shown in SEQ ID NOs: 78 and 79. In some embodiments the antigen-binding molecule contacts one or more amino acid residues of the regions of HER3 shown in SEQ ID NOs: 78 and 79. In some embodiments, the antigen-binding molecule binds to the region of HER3 shown in SEQ ID NO: 78. In some embodiments the antigen-binding molecule contacts one or more amino acid residues of the region of HER3 shown in SEQ ID NO:78. In some embodiments, the antigen-binding molecule binds to the region of HER3 shown in SEQ ID NO:79. In some embodiments the antigen-binding molecule contacts one or more amino acid residues of the region of HER3 shown in SEQ ID NO:79.

In some embodiments, the antigen-binding molecule does not bind to the region of HER3 corresponding to positions 260 to 279 of SEQ ID NO:1. In some embodiments the antigen-binding molecule does not contact an amino acid residue of the region of HER3 corresponding to positions 260 to 279 of SEQ ID NO: 1.

The region of a peptide/polypeptide to which an antibody binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3):145-156, which is hereby incorporated by reference in its entirety.

In some embodiments the antigen-binding molecule is capable of binding the same region of HER3, or an overlapping region of HER3, to the region of HER3 which is bound by an antibody comprising the VH and VL sequences of one of antibody clones 10D1_c89, 10D1, 10D1_c75, 10D1_c76, 10D1_c77, 10D1_c78v1, 10D1_c78v2, 10D1_11B, 10D1_c85v1, 10D1_c85v2, 10D1_c8501, 10D1_c8502, 10D1_c87, 10D1_c90, 10D1_c91, 10D1_c92 and 10D1_c93 described herein. In some embodiments the antigen-binding molecule is capable of binding the same region of HER3, or an overlapping region of HER3, to the region of HER3 which is bound by an antibody comprising the VH and VL sequences of antibody clone 10D1_c89.

In some embodiments, the antigen-binding molecule is capable of binding to a polypeptide comprising, or consisting of, the amino acid sequence of one of SEQ ID NOs: 1, 3, 4, 6 or 8. In some embodiments, the antigen-binding molecule is capable of binding to a polypeptide comprising, or consisting of, the amino acid sequence of SEQ ID NO:9. In some embodiments, the antigen-binding molecule is capable of binding to a polypeptide comprising, or consisting of, the amino acid sequence of SEQ ID NO:16. In some embodiments, the antigen-binding molecule is capable of binding to a peptide/polypeptide comprising, or consisting of, the amino acid sequence of SEQ ID NO:77. In some embodiments, the antigen-binding molecule is capable of binding to a peptide/polypeptide comprising, or consisting of, the amino acid sequences of SEQ ID NOs: 78 and 79. In some embodiments, the antigen-binding molecule is capable of binding to a peptide/polypeptide comprising, or consisting of, the amino acid sequence of SEQ ID NO:78. In some embodiments, the antigen-binding molecule is capable of binding to a peptide/polypeptide comprising, or consisting of, the amino acid sequence of SEQ ID NO:79.

In some embodiments, the antigen-binding molecule is not capable of binding to a peptide consisting of the amino acid sequence corresponding to positions 260 to 279 of SEQ ID NO:1.

The ability of an antigen-binding molecule to bind to a given peptide/polypeptide can be analyzed by methods well known to the skilled person, including analysis by ELISA, immunoblot (e.g. western blot), immunoprecipitation, Surface Plasmon Resonance (SPR; see e.g. Hearty et al., Methods Mol Biol (2012) 907:411-442) or Bio-Layer Interferometry (see e.g. Lad et al., (2015) J Biomol Screen 20 (4): 498-507).

In embodiments where the antigen binding molecule is capable of binding to a peptide/polypeptide comprising a reference amino acid sequence, the peptide/polypeptide may comprise one or more additional amino acids at one or both ends of the reference amino acid sequence. In some embodiments the peptide/polypeptide comprises e.g. 1-5, 1-10, 1-20, 1-30, 1-40, 1-50, 5-10, 5-20, 5-30, 5-40, 5-50, 10-20, 10-30, 10-40, 10-50, 20-30, 20-40 or 20-50 additional amino acids at one or both ends of the reference amino acid sequence.

In some embodiments the additional amino acid(s) provided at one or both ends (i.e. the N-terminal and C-terminal ends) of the reference sequence correspond to the positions at the ends of the reference sequence in the context of the amino acid sequence of HER3.

In some embodiments the antigen-binding molecule is capable of binding to a peptide/polypeptide which is bound by an antibody comprising the VH and VL sequences of one of antibody clones 10D1_c89, 10D1, 10D1_c75, 10D1_c76, 10D1_c77, 10D1_c78v1, 10D1_c78v2, 10D1_11B, 10D1_c85v1, 10D1_c85v2, 10D1_c8501, 10D1_c8502, 10D1_c87, 10D1_c90, 10D1_c91, 10D1_c92 and 10D1_c93 described herein. In some embodiments the antigen-binding molecule is capable of binding to a peptide/polypeptide which is bound by an antibody comprising the VH and VL sequences of antibody clone 10D1_c89.

In some embodiments, the antigen-binding molecule comprises the CDRs of, or comprises the VH and VL of, a HER3-binding antibody clone selected from 10D1_c89, 10D1, 10D1_c75, 10D1_c76, 10D1_c77, 10D1_c78v1, 10D1_c78v2, 10D1_11B, 10D1_c85v1, 10D1_c85v2, 10D1_c8501, 10D1_c8502, 10D1_c87, 10D1_c90, 10D1_c91, 10D1_c92 and 10D1_c93.

In some embodiments, the antigen-binding molecule comprises:
(1) a VH region incorporating the following CDRs:
   HC-CDR1 having the amino acid sequence of SEQ ID NO:40
   HC-CDR2 having the amino acid sequence of SEQ ID NO:43
   HC-CDR3 having the amino acid sequence of SEQ ID NO:48,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:66
   LC-CDR2 having the amino acid sequence of SEQ ID NO:69
   LC-CDR3 having the amino acid sequence of SEQ ID NO:74;
   or a variant thereof in which one or two or three amino acids in one or more of LC-CDR1, LC-CDR2 or LC-CDR3 are substituted with another amino acid.
(2) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
or a variant thereof in which one or two or three amino acids in one or more of LC-CDR1, LC-CDR2 or LC-CDR3 are substituted with another amino acid.
(3) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:64
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(4) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:65
   LC-CDR2 having the amino acid sequence of SEQ ID NO:67
   LC-CDR3 having the amino acid sequence of SEQ ID NO:71;
   or a variant thereof in which one or two or three amino acids in one or more of LC-CDR1, LC-CDR2 or LC-CDR3 are substituted with another amino acid.
(5) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(6) a VH region incorporating the following CDRs:
   HC-CDR1 having the amino acid sequence of SEQ ID NO:39
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(7) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(8) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:46,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(9) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:47,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(10) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:72;
   or a variant thereof in which one or two or three amino acids in one or more of LC-CDR1, LC-CDR2 or LC-CDR3 are substituted with another amino acid.
(11) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:73;
   or a variant thereof in which one or two or three amino acids in one or more of LC-CDR1, LC-CDR2 or LC-CDR3 are substituted with another amino acid.

In some embodiments, the antigen-binding molecule comprises:
(12) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:21; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:49.
(13) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:50.
(14) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:51.
(15) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:24; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:52.
(16) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:25; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(17) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:26; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(18) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:27; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(19) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:28; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(20) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:29; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(21) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:30; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:55.
(22) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:31; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:56.
(23) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:32; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:57.
(24) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:33; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:58.
(25) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:34; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:59.
(26) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:35; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:60.
(27) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:36; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:61.
(28) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:37; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:62.

In some embodiments, an antigen-binding molecule comprises, or consists of:
(29) (i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:75; and
   (ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:76.
      Antigen-Binding Molecules that Bind to EGFR

The present disclosure provides antigen-binding molecules that bind to EGFR.

In some embodiments, the antigen-binding molecule comprises the CDRs of an antigen-binding molecule which is capable of binding to EGFR. In some embodiments, the antigen-binding molecule comprises the FRs of an antigen-binding molecule which is capable of binding to EGFR. In some embodiments, the antigen-binding molecule comprises the CDRs and the FRs of an antigen-binding molecule which is capable of binding to EGFR. That is, in some embodiments the antigen-binding molecule comprises the VH region and the VL region of an antigen-binding molecule which is capable of binding to EGFR.

In some embodiments, an antigen-binding molecule which is capable of binding to EGFR according to the present disclosure may be selected from: any embodiment of an antigen-binding molecule described in U.S. Pat. No. 6,217,866 B1 (which is hereby incorporated by reference in its entirety), cetuximab (described e.g. in U.S. Pat. No. 6,217,866 B1 and Wong et al., Clin Ther. (2005) 27(6):684-694; DrugBank Acc. No. DB00002), panitumumab (described e.g. in Foon et al., Int J Radiat Oncol Biol Phys. (2004) 58(3):984-990; DrugBank Acc. No. DB01269), zalutumumab (described e.g. in Bastholt et al., Radiother Oncol. (2007) 85(1):24-28; DrugBank Acc. No. DB12202), necitumumab (described e.g. in Kuenen et al., Clin Cancer Res. (2010) 16(6):1915-1923; DrugBank Acc. No. DB09559), nimotuzumab (described e.g. in Ramakrishnan et al., mAbs (2009) 1(1):41-48; DrugBank Acc. No. DB06192), duligotuzumab (described e.g. in Fayette et al., Front Oncol. (2016) 6:232; DrugBank Acc. No. DB12142) and matuzumab (DrugBank Acc. No. DB05101). In some embodiments, the antigen-binding molecule is cetuximab.

In some embodiments the antigen-binding molecule is capable of binding the same region of EGFR, or an overlapping region of EGFR, to the region of EGFR which is bound by an antibody comprising the VH and VL sequences of cetuximab.

In some embodiments, the antigen-binding molecule comprises the CDRs of, or comprises the VH and VL of cetuximab.

In some embodiments, the antigen-binding molecule comprises:
(30) a VH region incorporating the following CDRs:
   HC-CDR1 having the amino acid sequence of SEQ ID NO:92
   HC-CDR2 having the amino acid sequence of SEQ ID NO:93
   HC-CDR3 having the amino acid sequence of SEQ ID NO:94,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:96
   LC-CDR2 having the amino acid sequence of SEQ ID NO:97
   LC-CDR3 having the amino acid sequence of SEQ ID NO:98;
   or a variant thereof in which one or two or three amino acids in one or more of LC-CDR1, LC-CDR2 or LC-CDR3 are substituted with another amino acid.

In some embodiments, the antigen-binding molecule comprises:
(31) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:91; and
   a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:95.

In some embodiments, an antigen-binding molecule comprises, or consists of:
(32) (i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:99; and
   (ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:100.

### Fc Regions

In some embodiments, the antigen-binding molecules of the present disclosure comprise an Fc region.

An Fc region is composed of CH2 and CH3 regions from one polypeptide, and CH2 and CH3 regions from another polypeptide. The CH2 and CH3 regions from the two polypeptides together form the Fc region.

Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

### Pharmaceutical Combinations and Compositions

The present disclosure provides a combination comprising (i) an antigen-binding molecule that binds to HER3, and (ii) an antigen-binding molecule that binds to EGFR. The present disclosure also provides a composition comprising (i) an antigen-binding molecule that binds to HER3, and (ii) an antigen-binding molecule that binds to EGFR.

In some aspects and embodiments, the combination further comprises (iii) a chemotherapeutic agent. In some aspects and embodiments, the composition further comprises (iii) a chemotherapeutic agent. Preferably, the chemotherapeutic agent is a microtubule-targeting agent, e.g. a taxane. More preferably the chemotherapeutic agent is a taxane, e.g. paclitaxel, docetaxel or cabazitaxel. Most preferably, the chemotherapeutic agent is docetaxel.

It will be appreciated that the antigen-binding molecule that binds to HER3 may be an antigen-binding molecule that binds to HER3 according to any embodiment described herein, and that similarly the antigen-binding molecule that binds to EGFR may be an antigen-binding molecule that binds to EGFR according to any embodiment described herein. In preferred embodiments of the combinations and compositions of the preceding paragraph, the antigen-binding molecule that binds to HER3 is selected from one of (1) to (29) above, and the antigen-binding molecule that binds to EGFR is selected from one of (30) to (32) above.

In some aspects and embodiments, the combination is a pharmaceutical combination. As used herein, a ‘pharmaceutical combination’ refers to a product that comprises plural (herein typically two or three) different active (i.e. therapeutic/prophylactic) agents, which are intended to be used in combination. The agents of a pharmaceutical combination may be formulated together or separately, but will typically be packaged together, typically with a package insert bearing instructions for the use of the agents in combination.

In some embodiments, the agents of a pharmaceutical combination are comprised in a single composition, e.g. a pharmaceutical composition comprising both/all agents. In some embodiments, the agents of a pharmaceutical combination are comprised in separate compositions; for example, the pharmaceutical combination may be provided as (i) a pharmaceutical composition comprising an antigen-binding molecule that binds to HER3, and (ii) a pharmaceutical composition comprising an antigen-binding molecule that binds to EGFR. In another example, the pharmaceutical combination may be provided as (i) a pharmaceutical composition comprising an antigen-binding molecule that binds to HER3, (ii) a pharmaceutical composition comprising an antigen-binding molecule that binds to EGFR, and (iii) a chemotherapeutic agent (e.g. docetaxel).

The present disclosure also provides compositions (e.g. pharmaceutical compositions and medicaments) comprising the agents described herein (i.e. antigen-binding molecules that bind to HER3, antigen-binding molecule that binds to EGFRs). Such compositions may comprise the relevant article in a formulation suitable for clinical use.

The compositions of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilizers, solubilizers, surfactants (e.g., wetting agents), masking agents or coloring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabilizer, solubilizer, surfactant, masking agent, coloring agent, flavoring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilizers, solubilizers, surfactants, masking agents, coloring agents, flavoring agents or sweetening agents can be found in standard pharmaceutical texts, for example, Remington's ‘The Science and Practice of Pharmacy’ (Ed. A. Adejare), 23rd Edition (2020), Academic Press.

The pharmaceutical compositions/medicaments according to the present disclosure may be formulated for administration to a subject, e.g. administration via a route of administration as appropriate for the nature of the composition/medicament and the disease/condition to be treated/prevented. In some embodiments, a pharmaceutical composition/medicament may be formulated for parenteral, systemic, topical, intracavitary, intravascular, intravenous, intra-arterial, intramuscular, intrathecal, intraocular, intraconjunctival, intratumoral, subcutaneous, intradermal, oral or transdermal administration. In some embodiments, a pharmaceutical composition/medicament may be formulated for administration by injection or infusion, or administration by ingestion.

Medicaments and pharmaceutical compositions may be formulated for administration to a blood vessel, or to a tissue/organ of interest (e.g. a tissue/organ affected by the disease/condition affected by the condition; e.g. a tissue/organ in which symptoms of the disease/condition manifest), or a tumor.

The pharmaceutical compositions/medicaments may comprise the agent(s) (i.e. the antigen-binding molecule that binds to HER3 and/or antigen-binding molecule that binds to EGFR) in a sterile or isotonic medium. The pharmaceutical compositions/medicaments may be provided in fluid, including gel, form. Fluid formulations may be formulated for administration by injection or infusion (e.g. via cannula) to a blood vessel, or a selected region of the human or animal body, or to a tumor. The pharmaceutical compositions/medicaments may be provided in solid form, e.g. in lyophilized form.

### Functional Properties

The combinations and compositions described herein may be characterized by reference to certain functional properties. In some embodiments, a combination/composition described herein may possess one or more of the following properties:
increases killing of cells expressing HER3 and/or EGFR;
increases ADCC of cells expressing HER3 and/or EGFR;
inhibits tumor growth and/or reduces tumor size/volume (e.g. of a HER3 and/or EGFR-expressing cancer);
increases survival of subjects having a cancer (e.g. a HER3 and/or EGFR-expressing cancer);
inhibits tumor growth and/or reduces tumor size/volume (e.g. of a HER3 and/or EGFR-expressing cancer), to an extent which is greater than the tumor growth inhibition/reduction in tumor size/volume observed when a constituent agent of the combination/composition is used alone;
increases survival of subjects having a cancer (e.g. a HER3 and/or EGFR-expressing cancer), to an extent which is greater than the increase in survival observed when a constituent agent of the combination/composition is used alone;
synergistically inhibits tumor growth and/or synergistically reduces tumor size/volume (e.g. of a HER3 and/or EGFR-expressing cancer), relative to the tumor growth inhibition/reduction in tumor size/volume observed when a constituent agent of the combination/composition is used alone; and/or
synergistically increases survival of subjects having a cancer (e.g. a HER3 and/or EGFR-expressing cancer), relative to the increase in survival observed when a constituent agent of the combination/composition is used alone.

It will be appreciated that a given combination/composition may display more than one of the properties recited in the preceding paragraph. A given combination/composition may be evaluated for the properties recited in the preceding paragraph using suitable assays. For example, the assays may be e.g. in vitro assays, optionally cell-based assays or cell-free assays. In some embodiments, the assays may be e.g. in vivo assays, i.e. performed in non-human animals. In some embodiments, the assays may be e.g. ex vivo assays, i.e. performed using cells/tissue/an organ obtained from a subject.

Where assays are cell-based assays, they may comprise treating cells with a combination/composition in order to determine whether the combination/composition displays one or more of the recited properties. Assays may employ species labelled with detectable entities in order to facilitate their detection. Assays may comprise evaluating the recited properties following treatment of cells separately with a range of quantities/concentrations of a given combination/composition (e.g. a dilution series).

Analysis of the results of such assays may comprise determining the concentration at which 50% of the maximal level of the relevant activity is attained. The concentration of a given agent at which 50% of the maximal level of the relevant activity is attained may be referred to as the ‘half-maximal effective concentration’ of the agent in relation to the relevant activity, which may also be referred to as the ‘EC₅₀’. Depending on the property, the EC₅₀ may also be referred to as the ‘half-maximal inhibitory concentration’ or ‘IC₅₀’, this being the concentration of the agent at which 50% of the maximal level of inhibition of a given property is observed.

In some embodiments, the combination/composition according to the present disclosure potentiates (i.e. upregulates, enhances) cell killing of cells comprising/expressing HER3 and/or EGFR.

In some embodiments, a combination/composition according to the present disclosure may inhibit growth or reduce metastasis of a cancer comprising cells comprising/expressing HER3 and/or EGFR. In some embodiments, a combination/composition may potentiate (i.e. upregulate, enhance) cell killing of cells comprising/expressing HER3 and/or EGFR. In some embodiments, a combination/composition may inhibit growth of cells of a cancer, or may inhibit growth of a tumor, comprising cells comprising/expressing HER3 and/or EGFR. In some embodiments, a combination/composition may inhibit metastasis of a cancer/tumor comprising cells comprising/expressing HER3 and/or EGFR.

Cell killing can be investigated, for example, using any of the methods reviewed in Zaritskaya et al., Expert Rev Vaccines (2011), 9(6):601-616, hereby incorporated by reference in its entirety. Examples of in vitro assays of cytotoxicity/cell killing assays include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Cell killing of a given test cell type by a given effector immune cell type can be analyzed e.g. by co-culturing the test cells with the effector immune cells, and measuring the number/proportion of viable/dead (e.g. lysed) test cells after a suitable period of time. Other suitable assays include the xCELLigence real-time cytolytic in vitro potency assay described in Cerignoli et al., PLOS One. (2018) 13(3): e0193498 (hereby incorporated by reference in its entirety).

In some embodiments a combination/composition according to the present disclosure is capable of reducing the number/proportion of cells expressing HER3 and/or EGFR. In some embodiments, a combination/composition according to the present disclosure is capable of depleting/enhancing depletion of such cells.

The constituent antigen-binding molecules of a combination/composition according to the present disclosure may comprise one or more moieties for potentiating a reduction in the number/proportion of cells expressing HER3 and/or EGFR. For example, the antigen-binding molecules may e.g. comprise an Fc region and/or a drug moiety.

Fc regions provide for interaction with Fc receptors and other molecules of the immune system to bring about functional effects. IgG Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein C1q, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

In some embodiments, antigen-binding molecules of a combination/composition according to the present disclosure comprises an Fc region capable of potentiating/directing one or more of ADCC, ADCP, CDC against, and/or potentiating formation of a MAC on or cell degranulation of, a cell expressing HER3 and/or EGFR (e.g. a cell expressing HER3 and/or EGFR at the cell surface).

In some embodiments, antigen-binding molecules of a combination/composition according to the present disclosure are capable of potentiating/directing ADCC against a cell expressing HER3 and/or EGFR.

The ability of, and extent to which, a given antigen-binding molecule is able to induce ADCC of a given target cell type can be analyzed e.g. according to the method described in Yamashita et al., Scientific Reports (2016) 6:19772 (hereby incorporated by reference in its entirety), or by ⁵¹Cr release assay as described e.g. in Jedema et al., Blood (2004) 103:2677-82 (hereby incorporated by reference in its entirety). The ability of, and extent to which, a given antigen-binding molecule is able to induce ADCP can be analyzed e.g. according to the method described in Kamen et al., J Immunol (2017) 198 (1 Supplement) 157.17 (hereby incorporated by reference in its entirety). The ability of, and extent to which, a given antigen-binding molecule is able to induce CDC can be analyzed e.g. using a C1q binding assay, e.g. as described in Schlothauer et al., Protein Engineering, Design and Selection (2016), 29(10): 457-466 (hereby incorporated by reference in its entirety).

In some embodiments, a combination/composition of the present disclosure displays anticancer activity. In some embodiments, the combination/composition increases killing of cancer cells. In some embodiments, the combination/composition causes a reduction in the number of cancer cells in vivo, e.g. as compared to an appropriate control condition. The cancer may be a cancer as described herein, e.g. a cancer expressing/overexpressing HER3 and/or EGFR.

In some embodiments, a combination/composition according to the present disclosure reduces/inhibits growth of a cancer and/or of a tumor of a cancer. In some embodiments, a combination/composition reduces tissue invasion by cells of a cancer. In some embodiments, a combination/composition reduces metastasis of a cancer. In some embodiments, a combination/composition displays anticancer activity. In some embodiments, a combination/composition reduces the growth/proliferation of cancer cells. In some embodiments, a combination/composition reduces the survival of cancer cells. In some embodiments, a combination/composition increases the killing of cancer cells. In some embodiments, a combination/composition of the present disclosure causes a reduction in the number of cancer cells e.g. in vivo. The cancer may be a cancer comprising cells expressing HER3 and/or EGFR.

A combination/composition of the present disclosure may be analyzed for the properties described in the preceding paragraph in appropriate assays. Such assays include e.g. in vivo models. By way of illustration, Example 2 herein describes the evaluation of tumor growth inhibition by the combination of the HER3-binding molecule HMBD-001 IgG1 and the EGFR-binding molecule cetuximab, in human cancer cell-derived models of various different cancers.

In some embodiments, administration of a combination/composition according to the present disclosure may cause one or more of: inhibition of the development/progression of the cancer, a delay to/prevention of onset of the cancer, a reduction in/delay to/prevention of tumor growth, a reduction in/delay to/prevention of tissue invasion, a reduction in/delay to/prevention of metastasis, a reduction in the severity of one or more symptoms of the cancer, a reduction in the number of cancer cells, a reduction in the cancer burden, a reduction in tumor size/volume, and/or an increase in survival of subjects having the cancer (e.g. progression free survival or overall survival), e.g. as determined in an appropriate model.

It will be appreciated that the properties recited in the preceding paragraph are evaluated after a period of time sufficient for an effect associated with treatment using the combination/composition to be observed. Tumor growth may be monitored by investigating tumor volume over time, e.g. as described in Example 2 herein. Tumor growth may be evaluated by measuring tumor volume (e.g. in mm³) over time.

In some embodiments, a combination/composition of the present disclosure is capable of reducing tumor size/volume (e.g. the mean tumor size/volume for the treatment group in an in vivo model, e.g. of a HER3 and/or EGFR-expressing cancer) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the tumor size/volume observed at the same time point in the absence of treatment with the combination/composition (or following treatment with an appropriate control composition known not to influence tumor growth), in a given assay. In some embodiments, evaluation of tumor size/volume for the purposes of such comparison is performed after more than 5 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first dose of the combination/composition, in the relevant model.

In some embodiments, a combination/composition of the present disclosure achieves a level of tumor growth inhibition (e.g. expressed as % tumor growth inhibition, e.g. calculated relative to tumor growth observed on treatment with isotype-matched control antibody) which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of tumor growth inhibition observed at the same time point in the absence of treatment with the combination/composition (or following treatment with an appropriate control composition known not to influence tumor growth), in a given assay. In some embodiments, evaluation of tumor growth inhibition for the purposes of such comparison is performed after more than 5 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first dose of the combination/composition, in the relevant model.

In some embodiments, a combination/composition of the present disclosure is capable of increasing median survival of subjects having a cancer (e.g. in an in vivo model, e.g. of a HER3 and/or EGFR-expressing cancer) to greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the median survival observed in the absence of treatment with the combination/composition (or following treatment with an appropriate control composition known not to influence survival of subjects having the cancer), in a given assay. Median survival may be expressed in days from the start of the experiment, for subjects in the relevant treatment groups.

In some embodiments, a combination/composition of the present disclosure reduces tumor growth, delays tumor growth, prevents tumor growth, reduces the severity of the symptoms of the cancer, reduces the number of cancer cells, reduces the cancer burden, reduces tumor size/volume and/or increases survival of subjects having the cancer to an extent which is greater than a component of the combination/composition employed alone. In some embodiments, a combination/composition reduces tumor growth, delays tumor growth, prevents tumor growth, reduces the severity of one or more symptoms of the cancer, reduces the number of cancer cells, reduces the cancer burden, reduces tumor size/volume and/or increases survival of subjects having the cancer to an extent which is greater than when the HER3-binding molecule constituent of the combination/composition is employed as a monotherapy, and/or to an extent which is greater than when the EGFR-binding molecule constituent of the combination/composition is employed as a monotherapy.

In some embodiments, a combination/composition of the present disclosure inhibits tumor growth and/or reduces tumor size/volume to an extent which is greater than the tumor growth inhibition/reduction in tumor size/volume observed when a component of the combination/composition is employed alone. In some embodiments, a combination/composition displays improved tumor growth inhibition and/or improved reduction of tumor size/volume as compared to the level observed when the HER3-binding molecule constituent of the combination/composition is employed as a monotherapy, and/or as compared to the level observed when the EGFR-binding molecule constituent of the combination/composition is employed as a monotherapy.

In some embodiments, a combination/composition of the present disclosure increases survival of subjects having a cancer to an extent which is greater than increase in survival observed when a component of the combination/composition is employed alone. In some embodiments, a combination/composition increases survival of subjects having a cancer as compared to the increase in survival observed when the HER3-binding molecule constituent of the combination/composition is employed as a monotherapy, and/or as compared to the increase in survival observed when the EGFR-binding molecule constituent of the combination/composition is employed as a monotherapy.

For the purposes of such comparisons, the monotherapy preferably employs the same dose of the relevant agent as is employed in the combination therapy. By way of illustration, in the experiments described in Example 2 herein, HMBD-001 IgG1 (anti-HER3 antibody) is administered as a monotherapy at 20 mg/kg bodyweight, per dose, and in combination therapy with cetuximab, HMBD-001 IgG1 is similarly administered at 20 mg/kg bodyweight, per dose. Likewise, cetuximab (anti-EGFR antibody) is administered as a monotherapy at 10 mg/kg bodyweight, per dose, and in combination therapy with HMBD-001 IgG1, cetuximab is similarly administered at 10 mg/kg bodyweight, per dose.

In some embodiments, a combination/composition of the present disclosure is capable of reducing tumor size/volume (e.g. the mean tumor size/volume for the treatment group in an in vivo model, e.g. of a HER3 and/or EGFR-expressing cancer) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the size/volume observed at the same time point following treatment of subjects with the same amount of one of the constituents of the combination/composition (i.e. the antigen-binding molecule that binds to HER3 or the antigen-binding molecule that binds to EGFR) as a monotherapy. In some embodiments, evaluation of tumor size/volume for the purposes of such comparison is performed after more than 5 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first dose of the combination/composition, in the relevant model.

In some embodiments, a combination/composition of the present disclosure achieves a level of tumor growth inhibition (e.g. expressed as % tumor growth inhibition, e.g. calculated relative to tumor growth observed on treatment with isotype-matched control antibody) which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of tumor growth inhibition observed following treatment of subjects with the same amount of one of the constituents of the combination/composition (i.e. the antigen-binding molecule that binds to HER3 or the antigen-binding molecule that binds to EGFR) as a monotherapy. In some embodiments, evaluation of tumor growth inhibition for the purposes of such comparison is performed after more than 5 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first dose of the combination/composition, in the relevant model.

In some embodiments, a combination/composition of the present disclosure is capable of increasing survival of subjects having a cancer (e.g. median survival of subjects having the cancer, e.g. as determined in an in vivo model, e.g. of a HER3 and/or EGFR-expressing cancer) to greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the survival observed following treatment of subjects with the same amount of one of the constituents of the combination/composition (i.e. the antigen-binding molecule that binds to HER3 or the antigen-binding molecule that binds to EGFR) as a monotherapy.

In the preceding three paragraphs, the ‘same amount’ refers to the quantity of the relevant agent employed in the combination/composition. By way of example, where subjects administered the combination are administered 20 mg/kg bodyweight of an antigen-binding molecule that binds to HER3 and 10 mg/kg bodyweight of antigen-binding molecule that binds to EGFR, monotherapy with the ‘same amount’ of the antigen-binding molecule that binds to HER3 is monotherapy with 20 mg/kg bodyweight of the antigen-binding molecule that binds to HER3. Similarly, monotherapy with the ‘same amount’ of the antigen-binding molecule that binds to EGFR is monotherapy with 10 mg/kg bodyweight of the antigen-binding molecule that binds to EGFR.

In some embodiments, a combination/composition of the present disclosure achieves a synergistic therapeutic and/or prophylactic effect. That is, in some embodiments, the combination/composition achieves a treatment effect that is synergistic (i.e. super-additive), relative to what is observed when the HER3-binding molecule constituent of the combination/composition is employed as a monotherapy, and/or relative to what is observed when the EGFR-binding molecule constituent of the combination/composition is employed as a monotherapy.

As used herein, a ‘synergistic’ or ‘super-additive’ level of a relevant effect (e.g. tumor growth inhibition, reduction in tumor size/volume, increase in survival) for a given combination/composition refers to a level of the effect which is greater than the sum of the effects observed for the individual components of the combination/composition.

Quantitative methods for assessing synergism are described e.g. in Tallarida, Genes Cancer. (2011) 2(11):1003-1008 and Chou, Cancer Res (2010) 70:440-446, both of which are hereby incorporated by reference in their entirety. Additive, synergistic and antagonistic effects may be evaluated in experiments in which a range of different doses of the combination/composition and the individual constituents thereof are evaluated for the relevant effect. Dose-response curves may be plotted, and evaluated in order to determine whether the combination/composition achieves a synergistic level of the relevant effect relative to the individual constituents of the combination/composition employed in isolation (i.e. as monotherapies). In some embodiments, synergy may be evaluated using combination index (CI) values calculated using the Chou-Talalay method described in Chou, Cancer Res (2010) 70:440-446. According to the Chou-Talalay method, for a given combination CI=1 indicates an additive effect, CI<1 indicates synergism, and CI≥1 indicates antagonism.

In some embodiments, a combination/composition of the present disclosure achieves a synergistic (i.e. super-additive) reduction in tumor growth, delay to tumor growth, prevention of tumor growth, reduction in the severity of one or more symptoms of the cancer, reduction in the number of cancer cells, reduction of the cancer burden, reduction of tumor size/volume and/or increase in survival of subjects having the cancer, relative to what is observed when either component of the combination/composition is employed alone. In some embodiments, a combination/composition achieves a synergistic (i.e. super-additive) reduction in tumor growth, delay to tumor growth, prevention of tumor growth, reduction in the severity of one or more symptoms of the cancer, reduction in the number of cancer cells, reduction of the cancer burden, reduction of tumor size/volume and/or increase in survival of subjects having the cancer, relative to what is observed when the HER3-binding molecule constituent of the combination/composition is employed as a monotherapy, and/or relative to what is observed when the EGFR-binding molecule constituent of the combination/composition is employed as a monotherapy.

### Therapeutic and Prophylactic Applications

The present disclosure provides methods and articles (e.g. the agents, combinations and compositions of the present disclosure) for the treatment and/or prevention of disease, e.g. cancers.

Accordingly, the present disclosure provides an antigen-binding molecule that binds to HER3 for use in a method of treating or preventing a cancer (e.g. a cancer described herein), wherein the method further comprises administering an antigen-binding molecule that binds to EGFR. Also provided is an antigen-binding molecule that binds to EGFR for use in a method of treating or preventing a cancer (e.g. a cancer described herein), wherein the method further comprises administering an antigen-binding molecule that binds to HER3.

Also provided is the use of an antigen-binding molecule that binds to HER3 in the manufacture of a medicament for use in a method of treating or preventing a cancer (e.g. a cancer described herein), wherein the method further comprises administering an antigen-binding molecule that binds to EGFR. Also provided is the use of an antigen-binding molecule that binds to EGFR in the manufacture of a medicament for use in a method of treating or preventing a cancer (e.g. a cancer described herein), wherein the method further comprises administering an antigen-binding molecule that binds to HER3.

Further provided is a method of treating or preventing a cancer (e.g. a cancer described herein), the method comprising administering a therapeutically- or prophylactically-effective amount of (i) an antigen-binding molecule that binds to HER3 and (ii) an antigen-binding molecule that binds to EGFR to a subject in need of treatment.

The present disclosure also provides (i) an antigen-binding molecule that binds to HER3 and (ii) an antigen-binding molecule that binds to EGFR for use in a method of treating or preventing a cancer (e.g. a cancer described herein) in a subject. Also provided is the use of (i) an antigen-binding molecule that binds to HER3 and (ii) an antigen-binding molecule that binds to EGFR in the manufacture of a medicament for use in treating or preventing a cancer (e.g. a cancer described herein) in a subject. Also provided is a method of treating or preventing a cancer (e.g. a cancer described herein) in a subject, comprising administering to the subject a therapeutically- or prophylactically-effective amount of (i) an antigen-binding molecule that binds to HER3 and (ii) an antigen-binding molecule that binds to EGFR.

In embodiments in accordance with aspects of the preceding paragraph, provision of (i) and (ii) may be as a combination therapy. In some embodiments, (i) and (ii) may be provided simultaneously or sequentially.

The present disclosure is directed to methods and articles (e.g. the agents, combinations and compositions of the present disclosure) for the treatment and/or prevention of cancer.

As used herein, a ‘cancer’ may be or comprise any unwanted cell proliferation (or any disease manifesting itself by unwanted cell proliferation), neoplasm or tumor. The cancer may be benign or malignant. The cancer may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue; examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

In some embodiments, the cancer to be treated/prevented comprises cells expressing an EGFR family member (e.g. HER3, EGFR, HER2 or HER4), and/or cells expressing a ligand for an EGFR family member. In some embodiments, the cancer to be treated/prevented is a cancer which is positive for an EGFR family member. In some embodiments, the cancer comprises cells that overexpress an EGFR family member and/or a ligand for an EGFR family member. Overexpression can be determined by detection of a level of expression which is greater than the level of expression by equivalent non-cancerous cells/non-tumor tissue.

Expression may be determined by any suitable means. Expression may be gene expression or protein expression. Gene expression can be determined e.g. by detection of mRNA encoding HER3, for example by quantitative real-time PCR (qRT-PCR). Protein expression can be determined e.g. by antibody-based methods, for example by western blot, immunohistochemistry, immunocytochemistry, flow cytometry, or ELISA.

In some embodiments the cancer is a cancer in which HER3 and/or EGFR is pathologically-implicated. That is, in some embodiments the cancer is a cancer which is caused or exacerbated by the expression of HER3 and/or EGFR, a cancer for which expression of HER3 and/or EGFR is a risk factor and/or a cancer for which expression of HER3 and/or EGFR is positively associated with onset, development, progression, severity or metastasis of the cancer. The cancer may be characterized by expression of HER3 and/or EGFR, e.g. the cancer may comprise cells (e.g. cells of tumor tissue) expressing HER3 and/or EGFR. Such cancers may be referred to as being positive for HER3 and/or EGFR. A cancer which is ‘positive’ for HER3 and/or EGFR may be a cancer comprising cells expressing HER3 and/or EGFR (e.g. at the cell surface). A cancer which is ‘positive’ for HER3 and/or EGFR may overexpress HER3 and/or EGFR.

In some embodiments, the cancer to be treated/prevented comprises cells harbouring a genetic variant (e.g. a mutation) which causes increased (gene and/or protein) expression and/or activity of HER3 and/or EGFR, relative to comparable cells harbouring a reference allele not comprising the genetic variant (e.g. a non-mutated, or ‘wildtype’ allele). The genetic variant may be or comprise insertion, deletion, substitution to, or larger-scale translocation/rearrangement of, the nucleotide sequence relative to the reference allele.

A mutation ‘resulting in’ increased expression of HER3 and/or EGFR may be known or predicted to cause, or may be associated with, increased gene/protein expression of HER3 and/or EGFR. A mutation ‘resulting in’ increased activity of HER3 and/or EGFR may be known or predicted to cause, or may be associated with, increased HER3-mediated signaling and/or EGFR-mediated signaling. Mutations resulting in increased expression and/or activity of HER3 and/or EGFR may be referred to as ‘activating’ mutations.

A mutation which causes increased expression of HER3 and/or EGFR may result in gene or protein expression of HER3 and/or EGFR which is not expressed by, and/or not encoded by genomic nucleic acid of, an equivalent cell not harbouring the mutation. That is, the HER3 and/or EGFR may be a neoantigen arising as a result of the mutation, and thus ‘increased expression’ may be from no expression.

A mutation which causes increased expression of HER3 and/or EGFR may result in increased gene or protein expression of HER3 and/or EGFR which is expressed by, and/or which is encoded by genomic nucleic acid of, an equivalent cell not comprising the mutation. By way of illustration, a cell may comprise a mutation resulting in an increase in the level of transcription of nucleic acid encoding HER3 and/or EGFR relative to the level of transcription of nucleic acid encoding HER3 and/or EGFR by an equivalent cell not comprising the mutation.

In some embodiments, a mutation which causes increased expression of HER3 and/or EGFR may cause an increase in gene expression of HER3 and/or EGFR relative to an equivalent cell not comprising the mutation. In some embodiments, a mutation which causes increased expression of HER3 and/or EGFR may cause an increase in protein expression of HER3 and/or EGFR relative to an equivalent cell not comprising the mutation.

In some embodiments, a mutation which causes increased expression of HER3 and/or EGFR may cause an increase in the level of HER3 and/or EGFR on or at the cell surface of a cell comprising the mutation, relative to an equivalent cell not comprising the mutation.

Cells having increased expression of HER3 and/or EGFR relative to the level of expression of HER3 and/or EGFR by a reference cell (e.g. as a result of mutation) may be described as ‘overexpressing’ HER3 and/or EGFR, or having ‘upregulated expression’ of HER3 and/or EGFR. For example, a cancer comprising cells harbouring a mutation resulting in increased expression of HER3 and/or EGFR relative to equivalent cells lacking the mutation may be described as a cancer comprising cells displaying overexpression/upregulated expression of HER3 and/or EGFR. In some embodiments, the reference cell lacking the mutation may be a non-cancerous cell (e.g. of equivalent cell type) or a cancerous cell (e.g. of equivalent cancer type).

A mutation which causes increased activity of HER3 and/or EGFR may result in an increase in HER3-mediated signaling and/or EGFR-mediated signaling relative to the level of HER3-mediated signaling and/or EGFR-mediated signaling by an equivalent cell not comprising the mutation.

In some embodiments, a cancer to be treated/prevented in accordance with the present disclosure may be characterized by an increase in the expression and/or activity of HER3 and/or EGFR (i.e. gene and/or protein expression) in an organ/tissue/subject affected by the disease/condition e.g. as compared to normal organ/tissue/subject (i.e. in the absence of the disease/condition). In some embodiments, cells and/or a tumor of a cancer to be treated/prevented may be characterized by an increase in the expression and/or activity of HER3 and/or EGFR, e.g. as compared to the level of expression and/or activity observed in equivalent non-cancerous cells/non-tumor tissue.

A HER3-overexpressing cancer may overexpress HER3 as a consequence of amplification of the HER3 gene. Similarly, an EGFR-overexpressing cancer may overexpress EGFR as a consequence of amplification of the EGFR gene.

In some embodiments, a cancer to be treated/prevented in accordance with the present disclosure is a HER3-amplified cancer. In some embodiments, the cancer is an EGFR-amplified cancer. In some embodiments, the cancer is a cancer comprising amplification of HER3 and EGFR. In some embodiments, a cancer to be treated/prevented in accordance with the present disclosure is a TP63-amplified cancer.

HER3, EGFR and/or TP63 amplification can be identified using techniques well known in the art, such as in situ hybridization. For example, HER3 amplification can be evaluated by fluorescence in situ hybridization, e.g. as described in Chung et al., J Gynecol Oncol. (2019) 30(5): e75. HER3-amplified cancers may comprise a ratio of 12q13.2 to chromosome 12 centromere≥2, as determined by ISH. EGFR amplification can similarly be evaluated by in situ hybridization, e.g. as described in French et al., Neuro-Oncology (2019) 21(10): 1263-1272. EGFR-amplified cancers may comprise a ratio of 7p11.2-7p12 to chromosome 7 centromere≥2, as determined by ISH. For example, TP63 amplification can be evaluated by fluorescence in situ hybridization, e.g. as described in Massion et al., Cancer Res. (2003) 63(21):7113-21. TP63-amplified cancers may comprise a ratio of 3q26-3qter to chromosome 3 centromere≥2, as determined by ISH.

EGFR and its association with and role in cancer is reviewed e.g. in Uribe et al., Cancers (Basel) (2021) 13(11): 2748, Sigismund et al., Mol Oncol. (2018) 12(1): 3-20 and da Silva Santos, et al., Int J Pharm. (2021) 592:120082, which is hereby incorporated by reference in its entirety. da Silva Santos, et al., Int J Pharm. (2021) 592:120082 describes intervention targeting EGFR for the treatment of cancer, including monoclonal anti-EGFR antibody therapy.

HER3 and its association with and role in cancer is reviewed e.g. in Mishra, et al., Oncol Rev. (2018) 12(1): 355, Karachaliou et al., BioDrugs. (2017) 31(1):63-73 and Zhang et al., Acta Biochimica et Biophysica Sinica (2016) 48(1): 39-48, all of which are hereby incorporated by reference in their entirety. Mishra, et al., Oncol Rev. (2018) 12(1): 355 also describes intervention targeting HER3 for the treatment of cancer, including monoclonal anti-HER3 antibody therapy.

In some embodiments, the cancer to be treated/prevented comprises cells expressing a ligand for HER3 (e.g. NRG1 and/or NRG2). In some embodiments, the cancer to be treated/prevented comprises cells expressing a level of expression of NRG1 and/or NRG2 which is greater than the level of expression by equivalent non-cancerous cells/non-tumor tissue. The cancer may be described as comprising cells that overexpress NRG1 and/or NRG2.

HER3-binding antigen-binding molecules described herein bind to HER3 with extremely high affinity when HER3 is bound by NRG (i.e. when HER3 is provided in the ‘open’ conformation), and also when HER3 is not bound by NRG (i.e. when HER3 is provided in the ‘closed’ conformation). Thus, they are particularly useful for the treatment/prevention of cancers characterized by HER3 ligand expression/overexpression, for example cancers/tumors comprising cells expressing/overexpressing a ligand for HER3.

In some embodiments, the cancer to be treated/prevented comprises cells harbouring a genetic variant (e.g. a mutation) which causes increased (gene and/or protein) expression of a ligand for HER3, relative to comparable cells harbouring a reference allele not comprising the genetic variant (e.g. a non-mutated, or ‘wildtype’ allele). The genetic variant may be or comprise insertion, deletion, substitution to, or larger-scale translocation/rearrangement of, the nucleotide sequence relative to the reference allele.

A mutation ‘resulting in’ increased expression of a ligand for HER3 may be known or predicted to cause, or may be associated with, increased gene/protein expression of a ligand for HER3. Mutations resulting in increased expression of a ligand for HER3 may be referred to as ‘activating’ mutations.

A mutation which causes increased expression of a ligand for HER3 may result in gene or protein expression of a ligand for HER3 which is not expressed by, and/or not encoded by genomic nucleic acid of, an equivalent cell not harbouring the mutation. That is, the ligand for HER3 may be a neoantigen arising as a result of the mutation, and thus ‘increased expression’ may be from no expression. By way of illustration, a cell comprising CD74-NRG1 gene fusion displays increased expression of the CD74-NRG1 fusion polypeptide encoded by the gene fusion relative to cells lacking the CD74-NRG1 gene fusion.

A mutation which causes increased expression of a ligand for HER3 may result in increased gene or protein expression of a ligand for HER3 which is expressed by, and/or which is encoded by genomic nucleic acid of, an equivalent cell not comprising the mutation. By way of illustration, a cell may comprise a mutation resulting in an increase in the level of transcription of nucleic acid encoding NRG1 relative to the level of transcription of nucleic acid encoding NRG1 by an equivalent cell not comprising the mutation.

In some embodiments, a mutation which causes increased expression of a ligand for HER3 may cause an increase in gene expression of a ligand for HER3 relative to an equivalent cell not comprising the mutation. In some embodiments, a mutation which causes increased expression of a ligand for HER3 may cause an increase in protein expression of a ligand for HER3 relative to an equivalent cell not comprising the mutation.

In some embodiments, a mutation which causes increased expression of a ligand for HER3 may cause an increase in the level of a ligand for HER3 on or at the cell surface of a cell comprising the mutation, relative to an equivalent cell not comprising the mutation. In some embodiments, a mutation which causes increased expression of a ligand for HER3 may cause an increase in the level of a secretion of a ligand for HER3 from a cell comprising the mutation, relative to an equivalent cell not comprising the mutation.

Cells having increased expression of a ligand for HER3 relative to the level of expression of the ligand for HER3 by a reference cell (e.g. as a result of mutation) may be described as ‘overexpressing’ the ligand for HER3, or having ‘upregulated expression’ of the ligand for HER3. For example, a cancer comprising cells harbouring a mutation resulting in increased expression of a ligand for HER3 relative to equivalent cells lacking the mutation may be described as a cancer comprising cells displaying overexpression/upregulated expression of the ligand for HER3. In some embodiments, the reference cell lacking the mutation may be a non-cancerous cell (e.g. of equivalent cell type) or a cancerous cell (e.g. of equivalent cancer type).

Herein, a ‘ligand for HER3’ is generally intended to refer to a molecule capable of binding to HER3 through the ligand binding region of HER3 formed by domains I and III of HER3. In some embodiments, a ligand for HER3 binds to HER3 via interaction with domains I and/or III of HER3. Exemplary ligands for HER3 include neuregulins such as NRG1 and NRG2, which bind to HER3 via interaction between their EGF-like domains and the ligand binding region of HER3.

The HER3 ligand is preferably able to bind and trigger signaling through the HER3 receptor and/or receptor complexes comprising HER3. As will be clear from the present disclosure, receptor complexes comprising HER3 may further comprise an interaction partner for HER3 as described herein, e.g. HER3, HER2, EGFR, HER4, HGFR, IGF1R and/or cMet.

In some embodiments the ligand for HER3 is able to bind to HER3 receptor/receptor complex expressed by a cell other than the cell having increased expression of the HER3 ligand. For example, in some embodiments the ligand for HER3 is able to bind to a HER3-expressing cancer cell.

In some embodiments the ligand for HER3 is able to bind to HER3 receptor/receptor complex expressed by the cell having increased expression of the HER3 ligand.

In some embodiments the cancer to be treated/prevented comprises (i) cells expressing HER3, and (ii) cells expressing a ligand for HER3 (e.g. having increased expression of a ligand for HER3, e.g. as a consequence of mutation resulting in increased expression of a ligand for HER3).

In some embodiments the cancer to be treated/prevented comprises cells which (i) express HER3 and (ii) which also express a ligand for HER3 (e.g. which have increased expression of a ligand for HER3, e.g. as a consequence of mutation resulting in increased expression of a ligand for HER3).

In some embodiments, the ligand for HER3 comprises, or consists of, the amino acid sequence of a HER3-binding region of a ligand for HER3, or an amino acid sequence derived from a HER3-binding region of a ligand for HER3. An amino acid sequence which is derived from a HER3-binding region of a ligand for HER3 may comprise at least 60% (e.g. 70%, 75%, 80%, 85%, 90%, 91%, 92%, 93%, 94%, 95%, 96%, 97%, 98%, 99% or 100%) amino acid sequence identity to the amino acid sequence from which it is derived.

In some embodiments, the ligand for HER3 comprises an EGF-like domain capable of binding to HER3, or a HER3-binding fragment thereof. In some embodiments, a HER3-binding EGF-like domain/fragment is, or is derived from, an EGF family member (e.g. heparin-binding EGF-like growth factor (HB-EGF), transforming growth factor-α (TGF-α), amphiregulin (AR), epiregulin (EPR), epigen, betacellulin (BTC), NRG1, NRG2, NRG3 or NRG4).

Exemplary ligands for HER3 include neuregulins (NRGs). Neuregulins include NRG1 (including alpha, alpha2b, and alpha3 isoforms thereof), NRG2, NRG3 and NRG4. In some embodiments, an NRG is selected from NRG1, NRG2, NRG3 and NRG4. In some embodiments, an NRG is selected from NRG1 and NRG2.

The EGF-like domain of human NRG1, through which it binds to HER3, is formed by positions 178-222 of UniProt: Q02297-1. The EGF-like domain of human NRG2 is formed by positions 341-382 of UniProt: 014511-1. The EGF-like domain of human NRG3 is formed by positions 286-329 of UniProt: B9EGV5-1. The EGF-like domain of human NRG4 is formed by positions 5-46 of UniProt: Q8WWG1-1. In some embodiments, an EGF-like domain/fragment comprises, or consists of, an amino acid sequence having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of an NRG (NRG1, NRG2, NRG3 or NRG4).

In some embodiments a ligand for HER3 is not an EGFR family protein (e.g. HER3, HER2, EGFR, HER4, HGFR, IGF1R, cMet).

In some embodiments, the mutation resulting in increased expression of a ligand for HER3 is an NRG gene fusion. In some embodiments, the ligand for HER3 is the product of (i.e. a polypeptide encoded by) an NRG gene fusion. In some embodiments the cancer comprises cells having an NRG gene fusion. As used herein, an ‘NRG gene fusion’ refers to a genetic variant encoding a polypeptide comprising (i) an amino acid sequence of an NRG protein (e.g. NRG1, NRG2, NRG3 or NRG4; e.g. NRG1 or NRG2), and (ii) an amino acid sequence of a protein other than the NRG protein.

It will be appreciated that an NRG gene fusion preferably encodes a HER3 ligand as described herein. In some embodiments, an NRG gene fusion encodes a polypeptide comprising a HER3-binding region of an NRG protein. In some embodiments, an NRG gene fusion encodes a polypeptide comprising the EGF-like domain of an NRG protein, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of an NRG protein.

In some embodiments, an NRG gene fusion encodes a fusion polypeptide comprising a transmembrane domain. In some embodiments, an NRG gene fusion encodes a fusion polypeptide comprising the transmembrane domain of a protein other than the NRG protein.

In some embodiments, an NRG gene fusion is an NRG1 gene fusion. In some embodiments, the NRG1 gene fusion encodes a polypeptide comprising the EGF-like domain of NRG1, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of NRG1.

NRG1 gene fusions are described e.g. in WO 2021/048274 A1, WO 2018/182422 A1, WO 2019/051155 A1, Dhanasekaran et al., Nat Commun. (2014) 5:5893, Drilon et al., Cancer Discov. (2018) 8(6): 686-695, Nagasaka et al., Journal of Thoracic Oncology (2019) 14(8): 1354-1359 and Jonna et al., Clin Cancer Res. (2019) 25(16): 4966-4972, all of which are hereby incorporated by reference in their entirety. The diversity of NRG1 gene fusions may result from NRG1 being located on chromosome 8, which is particularly susceptible to genomic translocation events (Adélaïde et al., Genes Chromosomes Cancer. (2003) 37 (4): 333-45).

In some embodiments, an NRG1 gene fusion is selected from CLU-NRG1, CD74-NRG1, DOC4-NRG1, SLC3A2-NRG1, RBPMS-NRG1, WRN-NRG1, SDC4-NRG1, RAB2IL1-NRG1, VAMP2-NRG1, KIF13B-NRG1, THAP7-NRG1, SMAD4-NRG1, MDK-NRG1, TNC-NRG1, DIP2B-NRG1, MRPL13-NRG1, PARP8-NRG1, ROCK1-NRG1, DPYSL2-NRG1, ATP1B1-NRG1, CDH6-NRG1, APP-NRG1, AKAP13-NRG1, THBS1-NRG1, FOXA1-NRG1, PDE7A-NRG1, RAB3IL1-NRG1, CDK1-NRG1, BMPRIB-NRG1, TNFRSF10B-NRG1, and MCPH1-NRG1. In some embodiments, an NRG1 gene fusion is CLU-NRG1.

CD74-NRG1 gene fusion is described e.g. in Fernandez-Cuesta et al. Cancer Discov. (2014) 4:415-22 and Nakaoku et al., Clin Cancer Res (2014) 20:3087-93. DOC4-NRG1 gene fusion is described e.g. in Liu et al., Oncogene. (1999) 18(50): 7110-4 and Wang et al., Oncogene. (1999) 18(41): 5718-21. SLC3A2-NRG1 gene fusion is described e.g. in Nakaoku et al., Clin Cancer Res (2014) 20:3087-93, Shin et al., Oncotarget (2016) 7:69450-65 and Shin et al., Mol Cancer Ther. (2018) 17(9):2024-2033. RBPMS-NRG1, WRN-NRG1, RAB2IL1-NRG1 and SDC4-NRG1 gene fusions are described e.g. in Dhanasekaran et al., Nat Commun. (2014) 5:5893. VAMP2-NRG1 gene fusion is described e.g. in Jung et al., J Thorac Oncol. (2015) 10(7):1107-11 and Shim et al., J Thorac Oncol. (2015) 10 (8): 1156-62. KIF13B-NRG1 gene fusion is described e.g. in Xia et al., Int J Surg Pathol. (2017) 25(3):238-240. SMAD4-NRG1, AKAP13-NRG1, THBS1-NRG1, FOXA1-NRG1, PDE7A-NRG1, RAB3IL1-NRG1 and THAP7-NRG1 gene fusions are described e.g. in Drilon et al., Cancer Discov. (2018) 8(6):686-695. MDK-NRG1, TNC-NRG1, DIP2B-NRG1, MRPL13-NRG1, PARP8-NRG1, ROCK1-NRG1 and DPYSL2-NRG1 gene fusions are described e.g. in Jonna et al., Clin Cancer Res. (2019) 25(16):4966-4972. ATP1B1-NRG1 gene fusion is described e.g. in Drilon et al., Cancer Discov. (2018) 8(6):686-695 and Jones et al., Annals of Oncology (2017) 28:3092-3097. CLU-NRG1 gene fusion is described e.g. in Drilon et al., Cancer Discov. (2018) 8(6):686-695 and Nagasaka et al., Journal of Thoracic Oncology (2019) 14(8):1354-1359.

In some embodiments, an NRG gene fusion is an NRG2 gene fusion. In some embodiments, the NRG2 gene fusion encodes a polypeptide comprising the EGF-like domain of NRG2, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of NRG2.

NRG2 gene fusions include SLC12A2-NRG2 described e.g. in WO 2021/048274 A1, WO 2015/093557 A1, and ZNF208-NRG2 described in Dupain et al., Mol Ther. (2019) 27 (1): 200-218.

A cancer comprising cells having a mutation which results in increased expression of a ligand for HER3 (e.g. comprising cells having an NRG gene fusion, e.g. an NRG1 gene fusion or an NRG2 gene fusion) can be any cancer described herein. In some embodiments, such cancer may be of tissues/cells derived from the lung, breast, head, neck, kidney, ovary, pancreas, prostate, uterus, gallbladder, colon, rectum, bladder, soft tissue or nasopharynx.

In some embodiments, a cancer comprising cells having a mutation which results in increased expression of a ligand for HER3 (e.g. comprising cells having an NRG gene fusion, e.g. an NRG1 gene fusion or an NRG2 gene fusion) is selected from: lung cancer, non-small cell lung cancer, lung adenocarcinoma, invasive mucinous lung adenocarcinoma, lung squamous cell carcinoma, breast cancer, breast carcinoma, breast invasive carcinoma, head and neck cancer, head and neck squamous cell carcinoma, renal cancer, renal clear cell carcinoma, ovarian cancer, ovarian serous cystadenocarcinoma, pancreatic cancer, pancreatic adenocarcinoma, pancreatic ductal adenocarcinoma, prostate cancer, prostate adenocarcinoma, endometrial cancer, uterine carcinosarcoma, gallbladder cancer, cholangiocarcinoma, colorectal cancer, metastatic colorectal cancer, bladder cancer, urothelial bladder cancer, sarcoma, soft tissue sarcoma, neuroendocrine tumor and neuroendocrine tumor of the nasopharynx.

In some embodiments, the cancer to be treated/prevented is lung cancer (e.g. non-small cell lung cancer, lung adenocarcinoma, invasive mucinous lung adenocarcinoma or lung squamous cell carcinoma) comprising cells having an NRG1 gene fusion.

It will be appreciated that in embodiments herein, cancers comprising cells having specified characteristics may be or comprise tumors comprising cells having those characteristics.

As is common in the art, a cancer/tumor comprising cells having specified characteristics may be referred to herein simply as a cancer/tumor having those characteristics. By way of illustration, a cancer/tumor comprising cells having an NRG1 gene fusion may be referred to simply as ‘a cancer/tumor comprising NRG1 gene fusion’, or ‘an NRG1 gene fusion cancer/tumor’.

In some embodiments, the cancer to be treated/prevented comprises mutation conferring resistance to treatment with an inhibitor of BRAF. In some embodiments, the mutation is mutation at BRAF V600. In some embodiments, the mutation is BRAF V600E or V600K. The cancer may be thyroid or colon cancer, e.g. RAS wildtype colorectal cancer. In some embodiments, the cancer to be treated/prevented comprises mutation conferring resistance to treatment with an inhibitor of BRAF (e.g. mutation at BRAF V600), and the treatment comprises administration of vemurafenib or darafenib.

In squamous cell cancer (SCC), the PI3K/AKT-signaling pathway is commonly altered by gene amplification and/or mutations. The frequently amplified 3q26/28 chromosomal region, in which PIK3CA is located, also resides the TP63 and SOX2 cell lineage genes. TP63 is a member of the TP53 gene family and is expressed in the basal compartment of the skin, esophagus, lung airways and larynx during development and homeostasis. TP63 is used as a diagnostic marker of squamous versus adenocarcinoma forms of lung and esophageal cancer. Preclinical data suggests that TP63 regulates NRG1 expression in SCC, suggesting that the HER3 signaling pathway is active in TP63-amplified squamous cell cancers. Furthermore, the association between high NRG1 level and response rate to an anti-HER3 antibody and the overexpression of EGFR in a subset of squamous cell cancers supports the rationale for combining 10D1F with cetuximab in EGFR-amplified squamous cell cancers.

In some embodiments, the cancer to be treated/prevented is a squamous cell cancer, i.e. a squamous cell carcinoma. In some embodiments, the cancer is an advanced or metastatic squamous cell carcinoma. In some embodiments, the squamous cell cancer is selected from: an EGFR-amplified squamous cell carcinoma, head and neck squamous cell carcinoma (HNSCC), lung squamous cell carcinoma (LUSC) and esophageal squamous cell carcinoma (ESCC), cervical squamous cell carcinoma, cutaneous squamous-cell carcinoma (cSCC), squamous cell thyroid carcinoma (SCTC), squamous cell carcinoma of the vagina (SCCV), squamous cell carcinoma of the prostate, and squamous cell carcinoma of the penis. In some embodiments, the squamous cell cancer is selected from: an EGFR-amplified squamous cell carcinoma, head and neck squamous cell carcinoma (HNSCC), lung squamous cell carcinoma (LUSC) and esophageal squamous cell carcinoma (ESCC).

In some embodiments, the cancer to be treated/prevented is an NRG1- and HER3-expressing/overexpressing cancer. In some embodiments, the cancer is an NRG1- and HER3-expressing/overexpressing squamous cell carcinoma. In some embodiments, the cancer is an NRG1- and HER3-expressing/overexpressing head and neck squamous cell carcinoma. In some embodiments, the cancer is an NRG1- and HER3-expressing/overexpressing esophageal squamous cell carcinoma. In some embodiments, the cancer is an NRG1- and HER3-expressing/overexpressing hypopharyngeal squamous cell carcinoma.

In some embodiments, the cancer to be treated/prevented is an EGFR-expressing/overexpressing cancer. In some embodiments, the cancer to be treated/prevented is an EGFR-expressing/overexpressing squamous cell carcinoma. In some embodiments, the cancer is an EGFR-expressing/overexpressing head and neck squamous cell carcinoma. In some embodiments, the cancer is an EGFR-expressing/overexpressing esophageal squamous cell carcinoma. In some embodiments, the cancer is an EGFR-expressing/overexpressing tongue squamous cell carcinoma.

In some embodiments, the cancer to be treated/prevented is an EGFR-expressing/overexpressing colorectal cancer. In some embodiments, the cancer to be treated/prevented is an EGFR-expressing/overexpressing colon adenocarcinoma.

In some embodiments, the cancer may be a relapsed cancer. As used herein, a ‘relapsed’ cancer refers to a cancer which responded to a treatment (e.g. a first line therapy for the cancer), but which has subsequently re-emerged/progressed, e.g. after a period of remission. For example, a relapsed cancer may be a cancer whose growth/progression was inhibited by a treatment (e.g. a first line therapy for the cancer), and which has subsequently grown/progressed.

In some embodiments, the cancer may be a refractory cancer. As used herein, a ‘refractory’ cancer refers to a cancer which has not responded to a treatment (e.g. a first line therapy for the cancer). For example, a refractory cancer may be a cancer whose growth/progression was not inhibited by a treatment (e.g. a first line therapy for the cancer). In some embodiments a refractory cancer may be a cancer for which a subject receiving treatment for the cancer did not display a partial or complete response to the treatment.

In some embodiments, a cancer is selected from: a cancer comprising cells expressing/overexpressing an EGFR family member (e.g. HER3, EGFR, HER2 or HER4), a cancer comprising cells expressing/overexpressing HER3, a cancer comprising cells expressing/overexpressing EGFR, a cancer comprising cells expressing/overexpressing HER3 and EGFR, a cancer comprising cells having a mutation resulting in increased expression of a ligand for HER3, a cancer comprising cells having a mutation resulting in increased expression of a ligand for EGFR, a cancer comprising cells having an NRG gene fusion, a solid tumor, a hematological cancer, a squamous cell cancer, an EGFR-amplified squamous cell carcinoma, breast cancer, breast carcinoma, breast invasive carcinoma, ductal carcinoma, metastatic breast cancer, triple-negative breast cancer, HER2-positive breast cancer, gastric cancer, gastric carcinoma, gastric adenocarcinoma, gastrointestinal adenocarcinoma, colorectal cancer, metastatic colorectal cancer, colon cancer, colorectal carcinoma, colorectal adenocarcinoma, colon adenocarcinoma, head and neck cancer, head and neck squamous cell carcinoma (HNSCC), lung cancer, non-small cell lung cancer, lung adenocarcinoma, invasive mucinous lung adenocarcinoma, lung squamous cell carcinoma (LUSC), ovarian cancer, ovarian carcinoma, ovarian serous adenocarcinoma, ovarian serous cystadenocarcinoma, fallopian tube cancer, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, oral cavity cancer, oropharyngeal cancer, esophageal cancer, esophageal squamous cell carcinoma (ESCC), esophageal adenocarcinoma, liver cancer, hepatocellular carcinoma, cholangiocarcinoma, gallbladder cancer, uterine cancer, endometrial cancer, uterine corpus endometrial carcinoma, uterine carcinosarcoma, thyroid cancer, thyroid cancer, thyroid carcinoma, pheochromocytoma, paraganglioma, bladder cancer, bladder urothelial carcinoma, prostate cancer, prostate adenocarcinoma, retinoblastoma, sarcoma, soft tissue sarcoma, peritoneal cancer, thymoma, neuroendocrine tumor, neuroendocrine tumor of the nasopharynx, squamous cell carcinoma of the skin, astrocytoma, low grade astrocytoma, high grade astrocytoma, anaplastic astrocytoma and glioblastoma multiforme.

In some embodiments, a cancer is selected from a squamous cell cancer or carcinoma (SCC). The SCC may originate from stratified squamous epithelium in any anatomical location. For example, the SCC may be non-melanoma skin cancer, head and neck cancer (HNSCC), esophageal cancer (ESCC), or non-small cell lung cancer (sqNSCLC).

In some embodiments, a cancer is selected from: a cancer comprising cells expressing/overexpressing HER3, a cancer comprising cells expressing/overexpressing EGFR, a cancer comprising cells expressing/overexpressing HER3 and EGFR, a squamous cell cancer, an EGFR-amplified squamous cell carcinoma, esophageal cancer, esophageal squamous cell carcinoma, head and neck cancer, head and neck squamous cell carcinoma, colorectal cancer, metastatic colorectal cancer, colon adenocarcinoma, pancreatic cancer, pancreatic adenocarcinoma, pancreatic ductal adenocarcinoma, lung cancer and lung squamous cell carcinoma.

Treatment of a cancer in accordance with the methods of the present disclosure achieves one or more of the following treatment effects: reduces the number of cancer cells in the subject, reduces the size of a cancerous tumor/lesion in the subject, inhibits (e.g. prevents or slows) growth of cancer cells in the subject, inhibits (e.g. prevents or slows) growth of a cancerous tumor/lesion in the subject, inhibits (e.g. prevents or slows) the development/progression of a cancer (e.g. to a later stage, or metastasis), reduces the severity of symptoms of a cancer in the subject, increases survival of the subject (e.g. progression free survival or overall survival), reduces a correlate of the number or activity of cancer cells in the subject, and/or reduces cancer burden in the subject.

Subjects may be evaluated in accordance with the Revised Criteria for Response Assessment: The Lugano Classification (described e.g. in Cheson et al., J Clin Oncol (2014) 32:3059-3068, incorporated by reference hereinabove) in order to determine their response to treatment. In some embodiments, treatment of a subject in accordance with the methods of the present disclosure achieves one of the following: complete response, partial response, or stable disease.

Prevention may refer to prevention of development of a cancer, and/or prevention of worsening of a cancer, e.g. prevention of progression of a cancer, e.g. to a later stage (e.g. metastasis).

In some embodiments, administration of a combination/composition according to the present disclosure may be associated with one or more of: inhibition of the development/progression of the cancer, a delay to/prevention of onset of the cancer, a reduction in/delay to/prevention of tumor growth, a reduction in/delay to/prevention of tissue invasion, a reduction in/delay to/prevention of metastasis, a reduction in the severity of one or more symptoms of the cancer, a reduction in the number of cancer cells, a reduction in the cancer burden, a reduction in tumor size/volume, and/or an increase in survival of subjects having the cancer (e.g. progression free survival or overall survival).

In accordance with various aspects of the present disclosure, a method of treating and/or preventing a cancer according to the present disclosure may comprise inhibiting the growth of a tumor, reducing the size/volume of a tumor and/or increasing the survival of a subject having the cancer.

In accordance with various aspects of the present disclosure, methods are provided which are for, or which comprise (e.g. in the context of treatment/prevention of a cancer, e.g. a cancer described herein), one or more of the following:
killing cells expressing HER3 and/or EGFR;
increasing ADCC of cells expressing HER3 and/or EGFR;
inhibiting tumor growth and/or reducing tumor size/volume (e.g. of a HER3 and/or EGFR-expressing cancer);
increasing survival of a subject having a cancer (e.g. a HER3 and/or EGFR-expressing cancer);
inhibiting tumor growth and/or reducing tumor size/volume (e.g. of a HER3 and/or EGFR-expressing cancer), to an extent which is greater than the inhibition of tumor growth/reduction of tumor size/volume observed when a constituent agent of the combination/composition is used alone;
increasing survival of a subject having a cancer (e.g. a HER3 and/or EGFR-expressing cancer), to an extent which is greater than the increase in survival observed when a constituent agent of the combination/composition is used alone;
synergistically inhibiting tumor growth and/or synergistically reducing tumor size/volume (e.g. of a HER3 and/or EGFR-expressing cancer), relative to the tumor growth inhibition/reduction in tumor size/volume observed when a constituent agent of the combination/composition is used alone; and/or
synergistically increasing survival of a subject having a cancer (e.g. a HER3 and/or EGFR-expressing cancer), relative to the increase in survival observed when a constituent agent of the combination/composition is used alone.

Also provided are agents according to the present disclosure for use in such methods, and the use of agents according to the present disclosure in the manufacture of compositions (e.g. medicaments) for use in such methods. It will be appreciated that the methods typically comprise administering an antigen-binding molecule that binds to HER3 and an antigen-binding molecule that binds to EGFR to a subject.

Similarly, one or more of the following may be observed in a subject following therapeutic or prophylactic intervention in accordance with the present disclosure (e.g. compared to the level/number/proportion etc. prior to intervention):
killing of cells expressing HER3 and/or EGFR;
increased ADCC of cells expressing HER3 and/or EGFR;
inhibition of tumor growth and/or reduction of tumor size/volume (e.g. of a HER3 and/or EGFR-expressing cancer);
increased survival of a subject having a cancer (e.g. a HER3 and/or EGFR-expressing cancer);
inhibition of tumor growth and/or reduction of tumor size/volume (e.g. of a HER3 and/or EGFR-expressing cancer), to an extent which is greater than the inhibition of tumor growth/reduction of tumor size/volume observed when a constituent agent of the combination/composition is used alone;
increased survival of a subject having a cancer (e.g. a HER3 and/or EGFR-expressing cancer), to an extent which is greater than the increase in survival observed when a constituent agent of the combination/composition is used alone;
synergistic inhibition of tumor growth and/or synergistic reduction of tumor size/volume (e.g. of a HER3 and/or EGFR-expressing cancer), relative to the tumor growth inhibition/reduction in tumor size/volume observed when a constituent agent of the combination/composition is used alone; and/or
synergistic increase in survival of a subject having a cancer (e.g. a HER3 and/or EGFR-expressing cancer), relative to the increase in survival observed when a constituent agent of the combination/composition is used alone.

In some embodiments, therapeutic/prophylactic intervention in accordance with the present disclosure may be described as being ‘associated with’ one or more of the effects described in the preceding paragraph. The skilled person is readily able to evaluate such properties using techniques that are routinely practiced in the art.

In some embodiments, therapeutic/prophylactic intervention with an antigen-binding molecule that binds to HER3 and an antigen-binding molecule that binds to EGFR in accordance with the present disclosure provides an improved treatment effect as compared to the effect observed when either agent is used as a monotherapy. In some embodiments, intervention with an antigen-binding molecule that binds to HER3 and an antigen-binding molecule that binds to EGFR provides a synergistic (i.e. super-additive) therapeutic and/or prophylactic effect as compared to the level of the relevant effect observed when either agent is used alone.

Administration of the agents, pharmaceutical combinations and pharmaceutical compositions of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (Ed. A. Adejare), 23rd Edition (2020), Academic Press.

Administration of the articles of the present disclosure may be e.g. parenteral, systemic, topical, intracavitary, intravascular, intravenous, intra-arterial, intramuscular, intrathecal, intraocular, intraconjunctival, intratumoral, subcutaneous, intradermal, oral or transdermal. Administration may be by injection, infusion or ingestion.

In some aspects and embodiments, articles of the present disclosure may be administered to a tissue/organ of interest (e.g. a tissue/organ affected by the disease/condition) affected by the condition (e.g. a tissue/organ in which symptoms of the disease/condition manifest). In some aspects and embodiments, articles of the present disclosure may be administered to the blood (i.e. intravenous/intra-arterial administration) by injection or infusion (e.g. via cannula), or may be administered subcutaneously or orally. In some aspects and embodiments, articles of the present disclosure may be administered to a tumor.

Where two or more agents (e.g. an antigen-binding molecule that binds to HER3 and an antigen-binding molecule that binds to EGFR) are administered in combination, the agents may be administered either simultaneously or sequentially.

Simultaneous administration refers to administration of the two or more agents (e.g. an antigen-binding molecule that binds to HER3 and an antigen-binding molecule that binds to EGFR) together, for example as a pharmaceutical composition containing both agents (i.e. as a combined preparation), or immediately after each other (e.g. within 1, 4, 6, 8 or 12 hours), and optionally via the same route of administration, e.g. to the same artery, vein or other blood vessel.

Sequential administration refers to administration of one of the agents followed after a given time interval by separate administration of another agent. It is not required that the agents are administered by the same route, although this is the case in some embodiments. The time interval may be any time interval.

In some embodiments, therapeutic or prophylactic intervention according to the present disclosure comprises: (i) administering an antigen-binding molecule that binds to HER3 to a subject having a cancer (e.g. a cancer as described herein), and (ii) administering to the subject an antigen-binding molecule that binds to EGFR. In some embodiments, (i) and (ii) are performed simultaneously. In some embodiments, (i) and (ii) are performed sequentially (e.g. (i) may be followed by (ii), or (ii) may be followed by (i)).

Multiple doses of the agents, pharmaceutical combinations and pharmaceutical compositions may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In some embodiments, therapeutic or prophylactic intervention according to the present disclosure may further comprise administering another agent for the treatment/prevention of the relevant disease/condition. For example, therapeutic or prophylactic intervention according to the present disclosure may further comprise administering a chemotherapeutic agent. In some embodiments, therapeutic or prophylactic intervention according to the present disclosure comprises: (i) administering an antigen-binding molecule that binds to HER3 to a subject having a cancer (e.g. a cancer as described herein), (ii) administering to the subject an antigen-binding molecule that binds to EGFR, and (iii) administering to the subject a chemotherapeutic agent. In some embodiments, two or more of (i), (ii), and (iii) are performed simultaneously (e.g. (i), (ii) and (iii) may be performed simultaneously, or (i) and (ii) may be performed simultaneously and (iii) may be performed sequentially, either before or after (i) and (ii)). In some embodiments, at least one of (i), (ii), and (iii) are performed sequentially (e.g. (i), (ii), and (iii) may be performed sequentially). For example, (i) may be followed by (ii) which in turn may be followed by (iii); (ii) may be followed by (i) which in turn may be followed by (iii); (iii) may be followed by (i) which in turn may be followed by (ii); or (iii) may be followed by (ii) which in turn may be followed by (i). Preferably, the chemotherapeutic agent is a microtubule-targeting agent, e.g. a taxane. More preferably the chemotherapeutic agent is a taxane, e.g. paclitaxel, docetaxel or cabazitaxel. Most preferably, the chemotherapeutic agent is docetaxel.

Chemotherapy refers to treatment of a cancer with a drug (a chemotherapeutic agent). The chemotherapeutic agent may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The chemotherapeutic agent may be formulated as a pharmaceutical composition or medicament. The formulation may comprise one or more chemotherapeutic agents together with one or more pharmaceutically acceptable diluents, excipients or carriers.

The chemotherapeutic agent may be administered by one or more routes of administration, e.g. parenteral, intravenous injection, oral, subcutaneous, intradermal, intraperitoneal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment. The treatment regime may indicate one or more of: the type of chemotherapy to administer to the patient; the dose of each drug; the time interval between administrations; the length of each treatment; the number and nature of any treatment holidays, if any etc. For a co-therapy a single treatment regime may be provided which indicates how each drug is to be administered.

Chemotherapeutic agents may be selected from: Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and lodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), lodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), [No Entries], Radium 223 Dichloride, Raloxifene Hydrochloride, Ramucirumab, Rasburicase, R-CHOP, RCVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and lodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VelP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) and Zytiga (Abiraterone Acetate).

### Subjects

The subject in accordance with aspects described herein may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer, e.g. a cancer described herein), may be suspected of having such a disease/condition, or may be at risk of developing/contracting such a disease/condition.

In some embodiments, the subject to be treated according to a therapeutic or prophylactic method of the present disclosure herein is a subject having, or at risk of developing, a cancer, e.g. a cancer described herein. In embodiments according to the present disclosure, a subject may be selected for treatment according to the methods based on characterization for certain markers of such disease/condition.

In some embodiments, a patient may be selected for treatment described herein based on the detection of a cancer expressing/overexpressing HER3 and/or EGFR, e.g. in a sample obtained from the subject (e.g. a biopsy, e.g. of a tumor).

### Kits

The present disclosure also provides kits of parts. A kit according to the present disclosure may comprise components for performing a method described herein, in whole or in part.

The kit may have at least one container having a predetermined quantity of a combination or composition described herein.

In some aspects of the present disclosure a kit of parts is provided. In some embodiments, the kit may comprise an antigen-binding molecule which binds to HER3 as described herein, and an antigen-binding molecule that binds to EGFR as described herein. The antigen-binding molecule which binds to HER3 and the antigen-binding molecule that binds to EGFR may be provided in a predetermined quantity. The antigen-binding molecule which binds to HER3 and the antigen-binding molecule that binds to EGFR may be provided in separate containers, or in the same container.

In some embodiments, the kit comprises a pharmaceutical combination or pharmaceutical composition according to the present disclosure.

The kit may provide the antigen-binding molecule which binds to HER3, antigen-binding molecule that binds to EGFR, pharmaceutical combination or pharmaceutical composition together with instructions for administration to a patient in order to treat a specified disease/condition (e.g. a disease/condition described herein, e.g. a cancer).

The kit may further comprise reagents, buffers and/or standards required for execution of a method according to the present disclosure. Kits according to the present disclosure may include instructions for use, e.g. in the form of an instruction booklet or leaflet. The instructions may include a protocol for performing any one or more of the methods described herein.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6 (298)) and MAFFT (Katoh and Standley 2013, Molecular Biology and Evolution, 30 (4) 772-780) software. When using such software, the default parameters, e.g. for gap penalty and extension penalty, are preferably used.

Sequences

| SEQ | | |
|---|---|---|
| ID NO: | DESCRIPTION | SEQUENCE |
| 1 | Human HER3 | MRANDALQVLGLLFSLARGSEVGNSQAVCPGTLNGLSVTGDAENQYQTLYKLYERCEVVMGNLEIVLTGHN |
| | isoform 1 | ADLSFLQWIREVTGYVLVAMNEFSTLPLPNLRVVRGTQVYDGKFAIFVMLNYNTNSSHALRQLRLTQLTEILS |
| | (UniProt: | GGVYIEKNDKLCHMDTIDWRDIVRDRDAEIVVKDNGRSCPPCHEVCKGRCWGPGSEDCQTLTKTICAPQCN |
| | P21860-1, v1) | GHCFGPNPNQCCHDECAGGCSGPQDTDCFACRHFNDSGACVPRCPQPLVYNKLTFQLEPNPHTKYQYGG |
| | | VCVASCPHNFVVDQTSCVRACPPDKMEVDKNGLKMCEPCGGLCPKACEGTGSGSRFQTVDSSNIDGFVN |
| | | CTKILGNLDFLITGLNGDPWHKIPALDPEKLNVFRTVREITGYLNIQSWPPHMHNFSVFSNLTTIGGRSLYNRG |
| | | FSLLIMKNLNVTSLGFRSLKEISAGRIYISANRQLCYHHSLNWTKVLRGPTEERLDIKHNRPRRDCVAEGKVC |
| | | DPLCSSGGCWGPGPGQCLSCRNYSRGGVCVTHCNFLNGEPREFAHEAECFSCHPECQPMEGTATQNGS |
| | | GSDTCAQCAHFRDGPHCVSSCPHGVLGAKGPIYKYPDVQNECRPCHENCTQGCKGPELQDCLGQTLVLIG |
| | | KTHLTMALTVIAGLVVIFMMLGGTFLYWRGRRIQNKRAMRRYLERGESIEPLDPSEKANKVLARIFKETELRK |
| | | LKVLGSGVFGTVHKGVWIPEGESIKIPVCIKVIEDKSGRQSFQAVTDHMLAIGSLDHAHIVRLLGLCPGSSLQL |
| | | VTQYLPLGSLLDHVRQHRGALGPQLLLNWGVQIAKGMYYLEEHGMVHRNLAARNVLLKSPSQVQVADFGV |
| | | ADLLPPDDKQLLYSEAKTPIKWMALESIHFGKYTHQSDVWSYGVTVWELMTFGAEPYAGLRLAEVPDLLEK |
| | | GERLAQPQICTIDVYMVMVKCWMIDENIRPTFKELANEFTRMARDPPRYLVIKRESGPGIAPGPEPHGLTNK |
| | | KLEEVELEPELDLDLDLEAEEDNLATTTLGSALSLPVGTLNRPRGSQSLLSPSSGYMPMNQGNLGESCQES |
| | | AVSGSSERCPRPVSLHPMPRGCLASESSEGHVTGSEAELQEKVSMCRSRSRSRSPRPRGDSAYHSQRHS |
| | | LLTPVTPLSPPGLEEEDVNGYVMPDTHLKGTPSSREGTLSSVGLSSVLGTEEEDEDEEYEYMNRRRRHSPP |
| | | HPPRPSSLEELGYEYMDVGSDLSASLGSTQSCPLHPVPIMPTAGTTPDEDYEYMNRQRDGGGPGGDYAA |
| | | MGACPASEQGYEEMRAFQGPGHQAPHVHYARLKTLRSLEATDSAFDNPDYWHSRLFPKANAQRT |
| 2 | Human HER3 | MRANDALQVLGLLFSLARGSEVGNSQAVCPGTLNGLSVTGDAENQYQTLYKLYERCEVVMGNLEIVLTGHN |
| | isoform 2 | ADLSFLQWIREVTGYVLVAMNEFSTLPLPNLRVVRGTQVYDGKFAIFVMLNYNTNSSHALRQLRLTQLTGQF |
| | (UniProt: | PMVPSGLTPQPAQDWYLLDDDPRLLTLSASSKVPVTLAAV |
| | P21860-2) | |
| 3 | Human HER3 | MRANDALQVLGLLFSLARGSEVGNSQAVCPGTLNGLSVTGDAENQYQTLYKLYERCEVVMGNLEIVLTGHN |
| | isoform 3 | ADLSFLQWIREVTGYVLVAMNEFSTLPLPNLRVVRGTQVYDGKFAIFVMLNYNTNSSHALRQLRLTQLTEILS |
| | (UniProt: | GGVYIEKNDKLCHMDTIDWRDIVRDRDAEIVVKDNGRSCPPCHEVCKGRCWGPGSEDCQTLTKTICAPQCN |
| | P21860-3) | GHCFGPNPNQCCHDECAGGCSGPQDTDCFACRHFNDSGACVPRCPQPLVYNKLTFQLEPNPHTKYQYGG |
| | | VCVASCPHNFVVDQTSCVRACPPDKMEVDKNGLKMCEPCGGLCPKAF |
| 4 | Human HER3 | MGNLEIVLTGHNADLSFLQWIREVTGYVLVAMNEFSTLPLPNLRVVRGTQVYDGKFAIFVMLNYNTNSSHAL |
| | isoform 4 | RQLRLTQLTEILSGGVYIEKNDKLCHMDTIDWRDIVRDRDAEIVVKDNGRSCPPCHEVCKGRCWGPGSEDC |
| | (UniProt: | QTLTKTICAPQCNGHCFGPNPNQCCHDECAGGCSGPQDTDCFACRHFNDSGACVPRCPQPLVYNKLTFQL |
| | P21860-4) | EPNPHTKYQYGGVCVASCPHNFVVDQTSCVRACPPDKMEVDKNGLKMCEPCGGLCPKACEGTGSGSRFQ |
| | | TVDSSNIDGFVNCTKILGNLDFLITGLNGDPWHKIPALDPEKLNVFRTVREITGYLNIQSWPPHMHNFSVFSNL |
| | | TTIGGRSLYNRGFSLLIMKNLNVTSLGFRSLKEISAGRIYISANRQLCYHHSLNWTKVLRGPTEERLDIKHNRP |
| | | RRDCVAEGKVCDPLCSSGGCWGPGPGQCLSCRNYSRGGVCVTHCNFLNGEPREFAHEAECFSCHPECQ |
| | | PMEGTATCNGSGSDTCAQCAHFRDGPHCVSSCPHGVLGAKGPIYKYPDVQNECRPCHENCTQGCKGPEL |
| | | QDCLGQTLVLIGKTHLTMALTVIAGLVVIFMMLGGTFLYWRGRRIQNKRAMRRYLERGESIEPLDPSEKANKV |
| | | LARIFKETELRKLKVLGSGVFGTVHKGVWIPEGESIKIPVCIKVIEDKSGRQSFQAVTDHMLAIGSLDHAHIVRL |
| | | LGLCPGSSLQLVTQYLPLGSLLDHVRQHRGALGPQLLLNWGVQIAKGMYYLEEHGMVHRNLAARNVLLKSP |
| | | SQVQVADFGVADLLPPDDKQLLYSEAKTPIKWMALESIHFGKYTHQSDVWSYGVTVWELMTFGAEPYAGLR |
| | | LAEVPDLLEKGERLAQPQICTIDVYMVMVKCWMIDENIRPTFKELANEFTRMARDPPRYLVIKRESGPGIAPG |
| | | PEPHGLTNKKLEEVELEPELDLDLDLEAEEDNLATTTLGSALSLPVGTLNRPRGSQSLLSPSSGYMPMNQGN |
| | | LGESCQESAVSGSSERCPRPVSLHPMPRGCLASESSEGHVTGSEAELQEKVSMCRSRSRSRSPRPRGDS |
| | | AYHSQRHSLLTPVTPLSPPGLEEEDVNGYVMPDTHLKGTPSSREGTLSSVGLSSVLGTEEEDEDEEYEYMN |
| | | RRRRHSPPHPPRPSSLEELGYEYMDVGSDLSASLGSTQSCPLHPVPIMPTAGTTPDEDYEYMNRQRDGGG |
| | | PGGDYAAMGACPASEQGYEEMRAFQGPGHQAPHVHYARLKTLRSLEATDSAFDNPDYWHSRLFPKANAQ |
| | | RT |
| 5 | Human HER3 | MALTVIAGLVVIFMMLGGTFLYWRGRRIQNKRAMRRYLERGESIEPLDPSEKANKVLARIFKETELRKLKVLG |
| | isoform 5 | SGVFGTVHKGVWIPEGESIKIPVCIKVIEDKSGRQSFQAVTDHMLAIGSLDHAHIVRLLGLCPGSSLQLVTQYL |
| | (UniProt: | PLGSLLDHVRQHRGALGPQLLLNWGVQIAKGMYYLEEHGMVHRNLAARNVLLKSPSQVQVADFGVADLLP |
| | P21860-5) | PDDKQLLYSEAKTPIKWMALESIHFGKYTHQSDVWSYGVTVWELMTFGAEPYAGLRLAEVPDLLEKGERLA |
| | | QPQICTIDVYMVMVKCWMIDENIRPTFKELANEFTRMARDPPRYLVIKRESGPGIAPGPEPHGLTNKKLEEVE |
| | | LEPELDLDLDLEAEEDNLATTTLGSALSLPVGTLNRPRGSQSLLSPSSGYMPMNQGNLGESCQESAVSGSS |
| | | ERCPRPVSLHPMPRGCLASESSEGHVTGSEAELQEKVSMCRSRSRSRSPRPRGDSAYHSQRHSLLTPVTP |
| | | LSPPGLEEEDVNGYVMPDTHLKGTPSSREGTLSSVGLSSVLGTEEEDEDEEYEYMNRRRRHSPPHPPRPS |
| | | SLEELGYEYMDVGSDLSASLGSTQSCPLHPVPIMPTAGTTPDEDYEYMNRQRDGGGPGGDYAAMGACPA |
| | | SEQGYEEMRAFQGPGHQAPHVHYARLKTLRSLEATDSAFDNPDYWHSRLFPKANAQRT |
| 6 | Mature human | SEVGNSQAVCPGTLNGLSVTGDAENQYQTLYKLYERCEVVMGNLEIVLTGHNADLSFLQWIREVTGYVLVA |
| | HER3 isoform 1 | MNEFSTLPLPNLRVVRGTQVYDGKFAIFVMLNYNTNSSHALRQLRLTQLTEILSGGVYIEKNDKLCHMDTIDW |
| | (UniProt: | RDIVRDRDAEIVVKDNGRSCPPCHEVCKGRCWGPGSEDCQTLTKTICAPQCNGHCFGPNPNQCCHDECA |
| | P21860-1, v1 | GGCSGPQDTDCFACRHFNDSGACVPRCPQPLVYNKLTFQLEPNPHTKYQYGGVCVASCPHNFVVDQTSC |
| | positions 20 to | VRACPPDKMEVDKNGLKMCEPCGGLCPKACEGTGSGSRFQTVDSSNIDGFVNCTKILGNLDFLITGLNGDP |
| | 1342) | WHKIPALDPEKLNVFRTVREITGYLNIQSWPPHMHNFSVFSNLTTIGGRSLYNRGFSLLIMKNLNVTSLGFRS |
| | | LKEISAGRIYISANRQLCYHHSLNWTKVLRGPTEERLDIKHNRPRRDCVAEGKVCDPLCSSGGCWGPGPGQ |
| | | CLSCRNYSRGGVCVTHCNFLNGEPREFAHEAECFSCHPECQPMEGTATCNGSGSDTCAQCAHFRDGPHC |
| | | VSSCPHGVLGAKGPIYKYPDVQNECRPCHENCTQGCKGPELQDCLGQTLVLIGKTHLTMALTVIAGLVVIFM |
| | | MLGGTFLYWRGRRIQNKRAMRRYLERGESIEPLDPSEKANKVLARIFKETELRKLKVLGSGVFGTVHKGVWI |
| | | PEGESIKIPVCIKVIEDKSGRQSFQAVTDHMLAIGSLDHAHIVRLLGLCPGSSLQLVTQYLPLGSLLDHVRQHR |
| | | GALGPQLLLNWGVQIAKGMYYLEEHGMVHRNLAARNVLLKSPSQVQVADFGVADLLPPDDKQLLYSEAKTP |
| | | IKWMALESIHFGKYTHQSDVWSYGVTVWELMTFGAEPYAGLRLAEVPDLLEKGERLAQPQICTIDVYMVMV |
| | | KCWMIDENIRPTFKELANEFTRMARDPPRYLVIKRESGPGIAPGPEPHGLTNKKLEEVELEPELDLDLDLEAE |
| | | EDNLATTTLGSALSLPVGTLNRPRGSQSLLSPSSGYMPMNQGNLGESCQESAVSGSSERCPRPVSLHPMP |
| | | RGCLASESSEGHVTGSEAELQEKVSMCRSRSRSRSPRPRGDSAYHSQRHSLLTPVTPLSPPGLEEEDVNG |
| | | YVMPDTHLKGTPSSREGTLSSVGLSSVLGTEEEDEDEEYEYMNRRRRHSPPHPPRPSSLEELGYEYMDVG |
| | | SDLSASLGSTQSCPLHPVPIMPTAGTTPDEDYEYMNRQRDGGGPGGDYAAMGACPASEQGYEEMRAFQG |
| | | PGHQAPHVHYARLKTLRSLEATDSAFDNPDYWHSRLFPKANAQRT |
| 7 | Mature human | SEVGNSQAVCPGTLNGLSVTGDAENQYQTLYKLYERCEVVMGNLEIVLTGHNADLSFLQWIREVTGYVLVA |
| | HER3 isoform 2 | MNEFSTLPLPNLRVVRGTQVYDGKFAIFVMLNYNTNSSHALRQLRLTQLTGQFPMVPSGLTPQPAQDWYLL |
| | (UniProt: | DDDPRLLTLSASSKVPVTLAAV |
| | P21860-2 | |
| | positions 20 to | |
| | 183) | |
| 8 | Mature human | SEVGNSQAVCPGTLNGLSVTGDAENQYQTLYKLYERCEVVMGNLEIVLTGHNADLSFLQWIREVTGYVLVA |
| | HER3 isoform 3 | MNEFSTLPLPNLRVVRGTQVYDGKFAIFVMLNYNTNSSHALRQLRLTQLTEILSGGVYIEKNDKLCHMDTIDW |
| | (UniProt: | RDIVRDRDAEIVVKDNGRSCPPCHEVCKGRCWGPGSEDCQTLTKTICAPQCNGHCFGPNPNQCCHDECA |
| | P21860-3 | GGCSGPQDTDCFACRHFNDSGACVPRCPQPLVYNKLTFQLEPNPHTKYQYGGVCVASCPHNFVVDQTSC |
| | positions 20 to | VRACPPDKMEVDKNGLKMCEPCGGLCPKAF |
| | 331) | |
| 9 | Human HER3 | SEVGNSQAVCPGTLNGLSVTGDAENQYQTLYKLYERCEVVMGNLEIVLTGHNADLSFLQWIREVTGYVLVA |
| | isoform 1 | MNEFSTLPLPNLRVVRGTQVYDGKFAIFVMLNYNTNSSHALRQLRLTQLTEILSGGVYIEKNDKLCHMDTIDW |
| | extracellular | RDIVRDRDAEIVVKDNGRSCPPCHEVCKGRCWGPGSEDCQTLTKTICAPQCNGHCFGPNPNQCCHDECA |
| | region (UniProt: | GGCSGPQDTDCFACRHFNDSGACVPRCPQPLVYNKLTFQLEPNPHTKYQYGGVCVASCPHNFVVDQTSC |
| | P21860-1, v1 | VRACPPDKMEVDKNGLKMCEPCGGLCPKACEGTGSGSRFQTVDSSNIDGFVNCTKILGNLDFLITGLNGDP |
| | positions 20 to | WHKIPALDPEKLNVFRTVREITGYLNIQSWPPHMHNFSVFSNLTTIGGRSLYNRGFSLLIMKNLNVTSLGFRS |
| | 643) | LKEISAGRIYISANRQLCYHHSLNWTKVLRGPTEERLDIKHNRPRRDCVAEGKVCDPLCSSGGCWGPGPGQ |
| | | CLSCRNYSRGGVCVTHCNFLNGEPREFAHEAECFSCHPECQPMEGTATCNGSGSDTCAQCAHFRDGPHC |
| | | VSSCPHGVLGAKGPIYKYPDVQNECRPCHENCTQGCKGPELQDCLGQTLVLIGKTHLT |
| 10 | Human HER3 | MALTVIAGLVVIFMMLGGTFL |
| | isoform 1 | |
| | transmembrane | |
| | domain (UniProt: | |
| | P21860-1, v1 | |
| | positions 644 to | |
| | 664) | |
| 11 | Human HER3 | YWRGRRIQNKRAMRRYLERGESIEPLDPSEKANKVLARIFKETELRKLKVLGSGVFGTVHKGVWIPEGESIKI |
| | isoform 1 | PVCIKVIEDKSGRQSFQAVTDHMLAIGSLDHAHIVRLLGLCPGSSLQLVTQYLPLGSLLDHVRQHRGALGPQL |
| | cytoplasmic | LLNWGVQIAKGMYYLEEHGMVHRNLAARNVLLKSPSQVQVADFGVADLLPPDDKQLLYSEAKTPIKWMALE |
| | domain (UniProt: | SIHFGKYTHQSDVWSYGVTVWELMTFGAEPYAGLRLAEVPDLLEKGERLAQPQICTIDVYMVMVKCWMIDE |
| | P21860-1, v1 | NIRPTFKELANEFTRMARDPPRYLVIKRESGPGIAPGPEPHGLTNKKLEEVELEPELDLDLDLEAEEDNLATTT |
| | positions 665 to | LGSALSLPVGTLNRPRGSQSLLSPSSGYMPMNQGNLGESCQESAVSGSSERCPRPVSLHPMPRGCLASES |
| | 1342) | SEGHVTGSEAELQEKVSMCRSRSRSRSPRPRGDSAYHSQRHSLLTPVTPLSPPGLEEEDVNGYVMPDTHL |
| | | KGTPSSREGTLSSVGLSSVLGTEEEDEDEEYEYMNRRRRHSPPHPPRPSSLEELGYEYMDVGSDLSASLG |
| | | STQSCPLHPVPIMPTAGTTPDEDYEYMNRQRDGGGPGGDYAAMGACPASEQGYEEMRAFQGPGHQAPH |
| | | VHYARLKTLRSLEATDSAFDNPDYWHSRLFPKANAQRT |
| 12 | Human HER3 | YWRGRRIQNKRAMRRYLERGESIEPLDPSEKANKVLARIFKETE |
| | isoform 1 | |
| | juxtamembrane | |
| | segment | |
| | (UniProt: | |
| | P21860-1, v1 | |
| | positions 665 to | |
| | 708) | |
| 13 | Human HER3 | LRKLKVLGSGVFGTVHKGVWIPEGESIKIPVCIKVIEDKSGRQSFQAVTDHMLAIGSLDH |
| | isoform 1 protein | AHIVRLLGLCPGSSLQLVTQYLPLGSLLDHVRQHRGALGPQLLLNWGVQIAKGMYYLEEH |
| | kinase domain | GMVHRNLAARNVLLKSPSQVQVADFGVADLLPPDDKQLLYSEAKTPIKWMALESIHFGKY |
| | (UniProt: | THQSDVWSYGVTVWELMTFGAEPYAGLRLAEVPDLLEKGERLAQPQICTIDVYMVMVKCW |
| | P21860-1, v1 | MIDENIRPTFKELANEFT |
| | positions 709 to | |
| | 966) | |
| 14 | Human HER3 | RMARDPPRYLVIKRESGPGIAPGPEPHGLTNKKLEEVELEPELDLDLDLEAEEDNLATTTLGSALSLPVGTLN |
| | isoform 1 C | RPRGSQSLLSPSSGYMPMNQGNLGESCQESAVSGSSERCPRPVSLHPMPRGCLASESSEGHVTGSEAEL |
| | terminal | QEKVSMCRSRSRSRSPRPRGDSAYHSQRHSLLTPVTPLSPPGLEEEDVNGYVMPDTHLKGTPSSREGTLS |
| | segment | SVGLSSVLGTEEEDEDEEYEYMNRRRRHSPPHPPRPSSLEELGYEYMDVGSDLSASLGSTQSCPLHPVPIM |
| | (UniProt: | PTAGTTPDEDYEYMNRQRDGGGPGGDYAAMGACPASEQGYEEMRAFQGPGHQAPHVHYARLKTLRSLE |
| | P21860-1, v1 | ATDSAFDNPDYWHSRLFPKANAQRT |
| | positions 967 to | |
| | 1342) | |
| 15 | Human HER3 | SEVGNSQAVCPGTLNGLSVTGDAENQYQTLYKLYERCEVVMGNLEIVLTGHNADLSFLQWIREVTGYVLVA |
| | extracellular | MNEFSTLPLPNLRVVRGTQVYDGKFAIFVMLNYNTNSSHALRQLRLTQLTEILSGGVYIEKNDKLCHMDTIDW |
| | region | RDIVRDRDAEIVVKDNGRSC |
| | subdomain I | |
| | (UniProt: | |
| | P21860-1, v1 | |
| | positions 20 to | |
| | 183) | |
| 16 | Human HER3 | PPCHEVCKGRCWGPGSEDCQTLTKTICAPQCNGHCFGPNPNQCCHDECAGGCSGPQDTDCFACRHFND |
| | extracellular | SGACVPRCPQPLVYNKLTFQLEPNPHTKYQYGGVCVASCPHNFVVDQTSCVRACPPDKMEVDKNGLKMC |
| | region | EPCGGLCPK |
| | subdomain II | |
| | (UniProt: | |
| | P21860-1, v1 | |
| | positions 184 to | |
| | 329) | |
| 17 | Human HER3 | ACEGTGSGSRFQTVDSSNIDGFVNCTKILGNLDFLITGLNGDPWHKIPALDPEKLNVFRTVREITGYLNIQSW |
| | extracellular | PPHMHNFSVFSNLTTIGGRSLYNRGFSLLIMKNLNVTSLGFRSLKEISAGRIYISANRQLCYHHSLNWTKVLR |
| | region | GPTEERLDIKHNRPRRDCVA |
| | subdomain III | |
| | (UniProt: | |
| | P21860-1, v1 | |
| | positions 330 to | |
| | 495) | |
| 18 | Human HER3 | EGKVCDPLCSSGGCWGPGPGQCLSCRNYSRGGVCVTHCNFLNGEPREFAHEAECFSCHPECQPMEGTA |
| | extracellular | TCNGSGSDTCAQCAHFRDGPHCVSSCPHGVLGAKGPIYKYPDVQNECRPCHENCTQGCKGPELQDCLGQ |
| | region | TLVLIGKTHLT |
| | subdomain IV | |
| | (UniProt: | |
| | P21860-1, v1 | |
| | positions 496 to | |
| | 643) | |
| 19 | Human HER3 | QPLVYNKLTFQLEPNPH |
| | extracellular | |
| | region | |
| | subdomain II | |
| | dimerization loop | |
| | (UniProt: | |
| | P21860-1, v1 | |
| | positions 261 to | |
| | 278) | |
| 20 | Rhesus | MGNLEIVLTGHNADLSFLQWIREVTGYVLVAMNEFSTLPLPNLRVVRGTQVYDGKFAIFVMLNYNTNSSHAL |
| | macaque HER3 | RQLRLTQLTEILSGGVYIEKNDKLCHMDTIDWKDIVRDQDAEIVVKDNGRSCPLCHEVCKGRCWGPGPEDC |
| | (UniProt: | QTLTKTICAPQCNGHCFGPNPNQCCHDECAGGCSGPQDTDCFACRHFNDSGACVPRCPQPLVYNKLTFQL |
| | F7HEH3-1, v2) | EPNPHTKYQYGGVCVASCPHNFVVDQTSCVRACPPDKMEVDKNGLKMCEPCGGLCPKACEGTGSGSRFQ |
| | | TVDSSNIDGFVNCTKILGNLDFLITGLNGDPWHKIPALDPEKLNVFRTVREITGYLNIQSWPPHMYNFSVFSNL |
| | | TTIGGRSLYNRGFSLLIMKNLNVTSLGFRSLKEISAGRIYISANRQLCYHHSLNWTKVLRGPTEERLDIKHNRP |
| | | RRDCVAEGKVCDPLCSSGGCWGPGPGQCLSCRNYSRGGVCVTHCNFLNGEPREFAHEAECFSCHPECQ |
| | | PMEGTATCNGSGSDTCAQCAHFRDGPHCVSSCPHGVLGAKGPIYKYPDVQNECRPCHENCTQGCKGPEL |
| | | QDCLGQTLVLIGKTHLTMALTVIAGLVVIFMMLGGTFLYWRGRRIQNKRAMRRYLERGESIEPLDPSEKANKV |
| | | LARIFKETELRKLKVLGSGVFGTVHKGVWIPEGESIKIPVCIKIIEDKSGRQSFQAVTDHMLAIGSLDHAHIVRLL |
| | | GLCPGSSLQLVTQYLPLGSLLDHVRQHRGALGPQLLLNWGVQIAKGMYYLEEHGMVHRNLAARNVLLKSPS |
| | | QVQVADFGVADLLPPDDKQLLYSEAKTPIKWMALESIHFGKYTHQSDVWSYGVTVWELMTFGAEPYAGLRL |
| | | AEVPDLLEKGERLAQPQICTIDVYMVMVKCWMIDENIRPTFKELANEFTRMARDPPRYLVIKRESGPGIAPGP |
| | | EPHGLTNKKLEEVELEPELDLDLDLEAEEDNLATTTLGSALSLPVGTLNRPRGSQSLLSPSSGYMPMNQGNL |
| | | GEAFQESAVSGSSEWCPRPVSLHPMPRGCLASESSEGHVTGSEAELQEKVSTCRSRSRSRSPRPRGDSA |
| | | YHSQRHSLLTPVTPLSPPGLEEEDVNGYVMPDTHLKGTPSSREGTLSSVGLSSVLGTEEEDEDEEYEYMNR |
| | | RRRHSPPRPPRPSSLEELGYEYMDVGSDLSASLGSTQSCPLHPVPVMPTAGTTPDEDYEYMNRQRGGSG |
| | | PGGDYAAMGACPASEQGYEEMRAFQGPGHQAPHVHYAHLKTLRSLEATDSAFDNPDYWHSRLFPKANAQ |
| | | RT |
| 21 | 10D1 heavy | DVQLQESGPDLVKPSQSLSLTCTVTGYSITSGYSWHWIRQFPGNKLEWMGSIHYSGGTNYNPSLKSRISITR |
| | chain variable | DTSKNQFFLQLNSVTTEDTATYFCARMTTAPRYPFDYWGQGTTLTVSS |
| | region | |
| 22 | 10D1_c75 heavy | DVQLQEWGAGLLKPSETLSLTCAVTGYSITSGYSWHWIRQFPGNGLEWIGSIHYSGGTNYNPTLKSRITISR |
| | chain variable | DTSKNQFSLKLSSVTAADTAVYFCARMTTAPRYPFDYWGQGTLVTVSS |
| | region | |
| 23 | 10D1_c76 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLKLSSVTAADTAVYFCARMTTAPRYPFDYWGQGTLVTVSS |
| | region | |
| 24 | 10D1_c77 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLKLSSVTAADTAVYFCARMTTAPRYPFDYWGQGTLVTVSS |
| | region | |
| 25 | 10D1_c78v1 | DVQLQEWGAGLLKPSETLSLTCAVTGYSITSGYSWHWIRQFPGNGLEWIGSIHYSGGTNYNPSLKSRITISR |
| | heavy chain | DTSKNQFSLKLSSVTAADTAVYFCARMTTAPRYPFDYWGQGTLVTVSS |
| | variable region | |
| 26 | 10D1_c78v2 | DVQLQEWGAGLLKPSETLSLTCAVTGYSITSGYSWHWIRQFPGKGLEWIGSIHYSGGTNYNPSLKSRITISRD |
| | heavy chain | TSKNQFSLKLSSVTAADTAVYFCARMTTAPRYPFDYWGQGTLVTVSS |
| | variable region | |
| 27 | 10D1_11B | DVQLQEWGAGLLKPSETLSLTCAVYGYSITSGYSWHWIRQPPGKGLEWIGSIHYSGGTNYNPSLKSRVTISR |
| | heavy chain | DTSKNQFSLKLSSVTAADTAVYYCARMTTAPRYPFDYWGQGTLVTVSS |
| | variable region | |
| 28 | 10D1_c85v1 | DVQLQEWGAGLLKPSETLSLTCAVTGYSITSGYSWHWIRQFPGNGLEWIGSIRYSGGTNYNPSLKSRITISR |
| | heavy chain | DTSKNQFSLKLGSVTAADTAVYFCARMTTAPRYPFDYWGQGTLVTVSS |
| | variable region | |
| 29 | 10D1_c85v2 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARMTTAPRYPFDYWGQGTLVTVSS |
| | variable region | |
| 30 | 10D1_c85o1 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARETTAPRYPFDYWGQGTLVTVSS |
| | variable region | |
| 31 | 10D1_c85o2 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARGTTAPRYPFDYWGQGTLVTVSS |
| | variable region | |
| 32 | 10D1_c87 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLRLSSVTAADTAVYFCARMTTAPRYPFDYWGQGTLVTVSS |
| | region | |
| 33 | 10D1_c89 heavy | QVQLQESGPGLVKPSQTLSLTCTVSGYSITSGYSWHWIRQHPGKGLEWIGSIRYSGGTDYNPSLKSLVTISA |
| | chain variable | DTSKNQFSLKLSSVTAADTAVYYCARMTTAPWYPFDYWGQGTTVTVSS |
| | region | |
| 34 | 10D1_c90 heavy | QVQLQESGPGLVKPSQTLFLTCTVSGYSITSGYSWHWIRQHPGKGLEWIGSIRYSGGTDYNPSLKSLVTISV |
| | chain variable | DTSKNQFSLKLSSVTAADTAVYYCARMTTAPWYPFDYWGQGTTVTVSS |
| | region | |
| 35 | 10D1_c91 heavy | QVQLQESGPGLVKPSQTLSLTCTVSGYYITSGYSWHWIRQHPGKGLEWIGSIRYSGGTDYNPSLKSLATISA |
| | chain variable | DTSKNQFSLKLSSVTAADTAVYYCARMTTAPWYPFDYWGQGTAVTVSS |
| | region | |
| 36 | 10D1_c92 heavy | DVQLQEWGAGLLKPSETLSLTCAVTGYSITSGYSWHWIRQFPGNGLEWIGSIHYSGGTNYNPTLKSRITISR |
| | chain variable | DTSKNQFSLKLSSVTAADTAVYFCARMTTAPRYPFDYWGQGTLVTVSS |
| | region | |
| 37 | 10D1_c93 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLRLSSVTAADTAVYFCARMTTAPRYPFDYWGQGTLVTVSS |
| | region | |
| 38 | 10D1, | GYSITSGYS |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c85v1, | |
| | 10D1_c85v2, | |
| | 10D1_c85o1, | |
| | 10D1_c85o2, | |
| | 10D1_c87, | |
| | 10D1_c89, | |
| | 10D1_c90, | |
| | 10D1_c92, | |
| | 10D1_c93 heavy | |
| | chain CDR1 | |
| 39 | 10D1_c91 heavy | GYYITSGYS |
| | chain CDR1 | |
| 40 | 10D1 derived | GYX₁ITSGYS |
| | consensus | wherein X₁ = S or Y |
| | heavy chain | |
| | CDR1 | |
| 41 | 10D1, | IHYSGGT |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c87, | |
| | 10D1_c92, | |
| | 10D1_c93 heavy | |
| | chain CDR2 | |
| 42 | 10D1_c85v1, | IRYSGGT |
| | 10D1_c85v2, | |
| | 10D1_c85o1, | |
| | 10D1_c85o2, | |
| | 10D1_c89, | |
| | 10D1_c90, | |
| | 10D1_c91 heavy | |
| | chain CDR2 | |
| 43 | 10D1 derived | IX₂YSGGT |
| | consensus | wherein X₂ = H or R |
| | heavy chain | |
| | CDR2 | |
| 44 | 10D1, | ARMTTAPRYPFDY |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c85v1, | |
| | 10D1_c85v2, | |
| | 10D1_c87, | |
| | 10D1_c92, | |
| | 10D1_c93 heavy | |
| | chain CDR3 | |
| 45 | 10D1_c89, | ARMTTAPWYPFDY |
| | 10D1_c90, | |
| | 10D1_c91 heavy | |
| | chain CDR3 | |
| 46 | 10D1_c85o1 | ARETTAPRYPFDY |
| | heavy chain | |
| | CDR3 | |
| 47 | 10D1_c85o2 | ARGTTAPRYPFDY |
| | heavy chain | |
| | CDR3 | |
| 48 | 10D1 derived | ARX₃TTAPX₄YPFDY |
| | consensus | wherein X₃ = M, E or G; X₄ = R or W |
| | heavy chain | |
| | CDR3 | |
| 49 | 10D1 light chain | DIVMTQSQKFMSTSVGDRVSVTCKASQIVGSNVAWYQQKPGQSPKPLIYSASYRYSGVPDRFTASGSGTDF |
| | variable region | TLTITNVQSEDLAEYFCQQYSSHPLTFGAGTKLELK |
| 50 | 10D1_c75 light | DIVMTQSPSSLSASVGDLVTITCKASQIVGSNVAWYQMKPGKSPKPLIYSASYLYFGVPSRFSGSGSGTDFT |
| | chain variable | LTISSLQPEDVAEYFCQQYSSHPLTFGPGTKVEIK |
| | region | |
| 51 | 10D1_c76 light | DIVMTQSPSSLSASGGDRVTITCKASQIVGYNVAWYQQKPGKSPKPLIYSASYLYSDVPSRFSASGSGTDFT |
| | chain variable | LTISSLQPEDVAEYFCQQYSSHPLTFGPGTKVEIK |
| | region | |
| 52 | 10D1_c77 light | VIVMTQSPSSLSASVGDRVTITCKASQIVGPNVAWYQQKPGKSPKPLIYSASYGYSDVPSRFSGSGSGTDFT |
| | chain variable | LTISSLQPEDVAEYFCQQYSTHPLTFGPGTKVEIK |
| | region | |
| 53 | 10D1_c78v1, | DIVMTQSPSSLSASVGDRVTITCKASQIVGSNVAWYQQKPGKSPKPLIYSASYGYSDVPSRFSGSGSGTDFT |
| | 10D1_c78v2, | LTISSLRPEDVATYYCQQYSSHPLTFGPGTKVEIK |
| | 10D1_11B light | |
| | chain variable | |
| | region | |
| 54 | 10D1_c85v1, | DIVMTQSPSSLSASVGDRVTITCKASQIVGSNVAWYQQKPGKSPKPLIYSARYQYSGVPFRFSGSGSGTDFT |
| | 10D1_c85v2 | LTISSLQPEDVATYYCQQYSSHPLTFGPGTKVEIK |
| | light chain | |
| | variable region | |
| 55 | 10D1_c85o1 | DIVMTQSPSSLSASVGDRVTITCKASQIVGSNVAWYQQKPGKSPKPLIYSARYQYSGVPFRFSGSGSGTDFT |
| | light chain | LTISSLQPEDVATYYCQQYSSHPLTFGPGTKVEIK |
| | variable region | |
| 56 | 10D1_c85o2 | DIVMTQSPSSLSASVGDRVTITCKASQIVGSNVAWYQQKPGKSPKPLIYSARYQYSGVPFRFSGSGSGTDFT |
| | light chain | LTISSLQPEDVATYYCQQYSSHPLTFGPGTKVEIK |
| | variable region | |
| 57 | 10D1_c87 light | DIVMTQSPSSLSASVGDRVTITCKASQIVGSNVAWYQQMPGKSPEPLIYSASYLYSDVPSRFSGSGSGTDFT |
| | chain variable | MTISSLQPEDVATYYCQQYSSHPLTFGPGTKVEIK |
| | region | |
| 58 | 10D1_c89 light | DIQMTQSPSSVSASVGDRVTITCKASQIVGSNVAWYQQKPGKAPEPLIYSASYLYSGVPSRFSGSGSGTDFT |
| | chain variable | LTISSLQPEDFATYYCQQYSSHPLTFGQGTKLEIK |
| | region | |
| 59 | 10D1_c90 light | DIQMTQSPSSVSASVGDRVTFTCKASQIVGSNVAWYQQKPGKAPEPLIYSASYLYSSVPSRFSGSGSGTEF |
| | chain variable | TMTISSLEPEDFATYYCQQYTTHPLTFGPGTKVEIK |
| | region | |
| 60 | 10D1_c91 light | DIQMTQSPSSVSASVGDRVTITCKASQIVGSNVAWYQQKPGKAPMPLIYSASYGYSGVPSRFSGSGSGTDF |
| | chain variable | TLTISSLQPEDFATYYCQQYSSHPLTFGQGTKLEIK |
| | region | |
| 61 | 10D1_c92 light | DIVMTQSPSSLSASVGDLVTITCKASQIVGSNVAWYQMKLGKSPKPLIYSASYLYFGVPSRFSGSGSGTDFTL |
| | chain variable | TISSLQPEDVAEYFCQQYFSHPLTFGPGTKVEIK |
| | region | |
| 62 | 10D1_c93 light | DIVMTQSPSSLSASVGDRVTITCKASQIVGSNVAWYQQKPGKSPKPLIYSASYLYSDVPSRFSGSGSGTDFT |
| | chain variable | MTISSLQPEDVATYYCQQYSSHPLTFGPGTKVEIK |
| | region | |
| 63 | 10D1, | QIVGSN |
| | 10D1_c75, | |
| | 10D1_c78v1, | |
| | 10D1_c78v2, | |
| | 10D1_11B, | |
| | 10D1_c85v1, | |
| | 10D1_c85v2, | |
| | 10D1_c85o1, | |
| | 10D1_c85o2, | |
| | 10D1_c87, | |
| | 10D1_c89, | |
| | 10D1_c90, | |
| | 10D1_c91, | |
| | 10D1_c92, | |
| | 10D1_c93 light | |
| | chain CDR1 | |
| 64 | 10D1_c76 light | QIVGYN |
| | chain CDR1 | |
| 65 | 10D1_c77 light | QIVGPN |
| | chain CDR1 | |
| 66 | 10D1 derived | QIVGX₅N |
| | consensus light | wherein X₅ = S, Y or P |
| | chain CDR1 | |
| 67 | 10D1, | SAS |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c87, | |
| | 10D1_c89, | |
| | 10D1_c90, | |
| | 10D1_c91, | |
| | 10D1_c92, | |
| | 10D1_c93 | |
| | light chain CDR2 | |
| 68 | 10D1_c85v1, | SAR |
| | 10D1_c85v2, | |
| | 10D1_c85o1, | |
| | 10D1_c85o2 | |
| | light chain CDR2 | |
| 69 | 10D1 derived | SAX₆ |
| | consensus light | wherein X₆ = S or R |
| | chain CDR2 | |
| 70 | 10D1, | QQYSSHPLT |
| | 10D1_c75, | |
| | 10D1_c76, | |
| | 10D1_c78v1, | |
| | 10D1_c78v2, | |
| | 10D1_11B, | |
| | 10D1_c85v1, | |
| | 10D1_c85v2, | |
| | 10D1_c85o1, | |
| | 10D1_c85o2, | |
| | 10D1_c87, | |
| | 10D1_c89, | |
| | 10D1_c91, | |
| | 10D1_c93 light | |
| | chain CDR3 | |
| 71 | 10D1_c77 light | QQYSTHPLT |
| | chain CDR3 | |
| 72 | 10D1_c90 light | QQYTTHPLT |
| | chain CDR3 | |
| 73 | 10D1_c92 light | QQYFSHPLT |
| | chain CDR3 | |
| 74 | 10D1 derived | QQYX₇X₈HPLT |
| | consensus light | wherein X₇ = S, T or F; X₈ = S or T |
| | chain CDR3 | |
| 75 | 10D1F hlgG1 | QVQLQESGPGLVKPSQTLSLTCTVSGYSITSGYSWHWIRQHPGKGLEWIGSIRYSGGTDYNPSLKSLVTISA |
| | HC | DTSKNQFSLKLSSVTAADTAVYYCARMTTAPWYPFDYWGQGTTVTVSSASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKSRWQQGNVFSCS |
| | | VMHEALHNHYTQKSLSLSPGK |
| 76 | 10D1F KLC | DIQMTQSPSSVSASVGDRVTITCKASQIVGSNVAWYQQKPGKAPEPLIYSASYLYSGVPSRFSGSGSGTDFT |
| | | LTISSLQPEDFATYYCQQYSSHPLTFGQGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVYACEVTHQGLSSPVTKSFNRGEC |
| 77 | HER3 binding | CFGPNPNQCCHDECAGGC |
| | site for 10D1- | |
| | derived clones | |
| 78 | Binding site | PNPNQ |
| | motif 1 | |
| 79 | Binding site | DECAG |
| | motif 2 | |
| 80 | Human EGFR | MRPSGTAGAALLALLAALCPASRALEEKKVCQGTSNKLTQLGTFEDHFLSLQRMFNNCEVVLGNLEITYVQR |
| | isoform 1 | NYDLSFLKTIQEVAGYVLIALNTVERIPLENLQIIRGNMYYENSYALAVLSNYDANKTGLKELPMRNLQEILHGA |
| | (UniProt: | VRFSNNPALCNVESIQWRDIVSSDFLSNMSMDFQNHLGSCQKCDPSCPNGSCWGAGEENCQKLTKIICAQ |
| | P00533-1, v1) | QCSGRCRGKSPSDCCHNQCAAGCTGPRESDCLVCRKFRDEATCKDTCPPLMLYNPTTYQMDVNPEGKYS |
| | | FGATCVKKCPRNYVVTDHGSCVRACGADSYEMEEDGVRKCKKCEGPCRKVCNGIGIGEFKDSLSINATNIK |
| | | HFKNCTSISGDLHILPVAFRGDSFTHTPPLDPQELDILKTVKEITGFLLIQAWPENRTDLHAFENLEIIRGRTKQ |
| | | HGQFSLAVVSLNITSLGLRSLKEISDGDVIISGNKNLCYANTINWKKLFGTSGQKTKIISNRGENSCKATGQVC |
| | | HALCSPEGCWGPEPRDCVSCRNVSRGRECVDKCNLLEGEPREFVENSECIQCHPECLPQAMNITCTGRGP |
| | | DNCIQCAHYIDGPHCVKTCPAGVMGENNTLVWKYADAGHVCHLCHPNCTYGCTGPGLEGCPTNGPKIPSIA |
| | | TGMVGALLLLLVVALGIGLFMRRRHIVRKRTLRRLLQERELVEPLTPSGEAPNQALLRILKETEFKKIKVLGSG |
| | | AFGTVYKGLWIPEGEKVKIPVAIKELREATSPKANKEILDEAYVMASVDNPHVCRLLGICLTSTVQLITQLMPF |
| | | GCLLDYVREHKDNIGSQYLLNWCVQIAKGMNYLEDRRLVHRDLAARNVLVKTPQHVKITDFGLAKLLGAEEK |
| | | EYHAEGGKVPIKWMALESILHRIYTHQSDVWSYGVTVWELMTFGSKPYDGIPASEISSILEKGERLPQPPICTI |
| | | DVYMIMVKCWMIDADSRPKFRELIIEFSKMARDPQRYLVIQGDERMHLPSPTDSNFYRALMDEEDMDDVVD |
| | | ADEYLIPQQGFFSSPSTSRTPLLSSLSATSNNSTVACIDRNGLQSCPIKEDSFLQRYSSDPTGALTEDSIDDTF |
| | | LPVPEYINQSVPKRPAGSVQNPVYHNQPLNPAPSRDPHYQDPHSTAVGNPEYLNTVQPTCVNSTFDSPAH |
| | | WAQKGSHQISLDNPDYQQDFFPKEAKPNGIFKGSTAENAEYLRVAPQSSEFIGA |
| 81 | Human EGFR | MRPSGTAGAALLALLAALCPASRALEEKKVCQGTSNKLTQLGTFEDHFLSLQRMFNNCEVVLGNLEITYVQR |
| | isoform 2 | NYDLSFLKTIQEVAGYVLIALNTVERIPLENLQIIRGNMYYENSYALAVLSNYDANKTGLKELPMRNLQEILHGA |
| | (UniProt: | VRFSNNPALCNVESIQWRDIVSSDFLSNMSMDFQNHLGSCQKCDPSCPNGSCWGAGEENCQKLTKIICAQ |
| | P21860-2) | QCSGRCRGKSPSDCCHNQCAAGCTGPRESDCLVCRKFRDEATCKDTCPPLMLYNPTTYQMDVNPEGKYS |
| | | FGATCVKKCPRNYVVTDHGSCVRACGADSYEMEEDGVRKCKKCEGPCRKVCNGIGIGEFKDSLSINATNIK |
| | | HFKNCTSISGDLHILPVAFRGDSFTHTPPLDPQELDILKTVKEITGLS |
| 82 | Human EGFR | MRPSGTAGAALLALLAALCPASRALEEKKVCQGTSNKLTQLGTFEDHFLSLQRMFNNCEVVLGNLEITYVQR |
| | isoform 3 | NYDLSFLKTIQEVAGYVLIALNTVERIPLENLQIIRGNMYYENSYALAVLSNYDANKTGLKELPMRNLQEILHGA |
| | (UniProt: | VRFSNNPALCNVESIQWRDIVSSDFLSNMSMDFQNHLGSCQKCDPSCPNGSCWGAGEENCQKLTKIICAQ |
| | P21860-3) | QCSGRCRGKSPSDCCHNQCAAGCTGPRESDCLVCRKFRDEATCKDTCPPLMLYNPTTYQMDVNPEGKYS |
| | | FGATCVKKCPRNYVVTDHGSCVRACGADSYEMEEDGVRKCKKCEGPCRKVCNGIGIGEFKDSLSINATNIK |
| | | HFKNCTSISGDLHILPVAFRGDSFTHTPPLDPQELDILKTVKEITGFLLIQAWPENRTDLHAFENLEIIRGRTKQ |
| | | HGQFSLAVVSLNITSLGLRSLKEISDGDVIISGNKNLCYANTINWKKLFGTSGQKTKIISNRGENSCKATGQVC |
| | | HALCSPEGCWGPEPRDCVSCRNVSRGRECVDKCNLLEGEPREFVENSECIQCHPECLPQAMNITCTGRGP |
| | | DNCIQCAHYIDGPHCVKTCPAGVMGENNTLVWKYADAGHVCHLCHPNCTYGPGNESLKAMLFCLFKLSSC |
| | | NQSNDGSVSHQSGSPAAQESCLGWIPSLLPSEFQLGWGGCSHLHAWPSASVIITASSCH |
| 83 | Human EGFR | MRPSGTAGAALLALLAALCPASRALEEKKVCQGTSNKLTQLGTFEDHFLSLQRMFNNCEVVLGNLEITYVQR |
| | isoform 4 | NYDLSFLKTIQEVAGYVLIALNTVERIPLENLQIIRGNMYYENSYALAVLSNYDANKTGLKELPMRNLQEILHGA |
| | (UniProt: | VRFSNNPALCNVESIQWRDIVSSDFLSNMSMDFQNHLGSCQKCDPSCPNGSCWGAGEENCQKLTKIICAQ |
| | P21860-4) | QCSGRCRGKSPSDCCHNQCAAGCTGPRESDCLVCRKFRDEATCKDTCPPLMLYNPTTYQMDVNPEGKYS |
| | | FGATCVKKCPRNYVVTDHGSCVRACGADSYEMEEDGVRKCKKCEGPCRKVCNGIGIGEFKDSLSINATNIK |
| | | HFKNCTSISGDLHILPVAFRGDSFTHTPPLDPQELDILKTVKEITGFLLIQAWPENRTDLHAFENLEIIRGRTKQ |
| | | HGQFSLAVVSLNITSLGLRSLKEISDGDVIISGNKNLCYANTINWKKLFGTSGQKTKIISNRGENSCKATGQVC |
| | | HALCSPEGCWGPEPRDCVSCRNVSRGRECVDKCNLLEGEPREFVENSECIQCHPECLPQAMNITCTGRGP |
| | | DNCIQCAHYIDGPHCVKTCPAGVMGENNTLVWKYADAGHVCHLCHPNCTYGS |
| 84 | Mature human | LEEKKVCQGTSNKLTQLGTFEDHFLSLQRMFNNCEVVLGNLEITYVQRNYDLSFLKTIQEVAGYVLIALNTVE |
| | EGFR isoform 1 | RIPLENLQIIRGNMYYENSYALAVLSNYDANKTGLKELPMRNLQEILHGAVRFSNNPALCNVESIQWRDIVSS |
| | | DFLSNMSMDFQNHLGSCQKCDPSCPNGSCWGAGEENCQKLTKIICAQQCSGRCRGKSPSDCCHNQCAA |
| | | GCTGPRESDCLVCRKFRDEATCKDTCPPLMLYNPTTYQMDVNPEGKYSFGATCVKKCPRNYVVTDHGSCV |
| | | RACGADSYEMEEDGVRKCKKCEGPCRKVCNGIGIGEFKDSLSINATNIKHFKNCTSISGDLHILPVAFRGDSF |
| | | THTPPLDPQELDILKTVKEITGFLLIQAWPENRTDLHAFENLEIIRGRTKQHGQFSLAVVSLNITSLGLRSLKEIS |
| | | DGDVIISGNKNLCYANTINWKKLFGTSGQKTKIISNRGENSCKATGQVCHALCSPEGCWGPEPRDCVSCRN |
| | | VSRGRECVDKCNLLEGEPREFVENSECIQCHPECLPQAMNITCTGRGPDNCIQCAHYIDGPHCVKTCPAGV |
| | | MGENNTLVWKYADAGHVCHLCHPNCTYGCTGPGLEGCPTNGPKIPSIATGMVGALLLLLVVALGIGLFMRR |
| | | RHIVRKRTLRRLLQERELVEPLTPSGEAPNQALLRILKETEFKKIKVLGSGAFGTVYKGLWIPEGEKVKIPVAIK |
| | | ELREATSPKANKEILDEAYVMASVDNPHVCRLLGICLTSTVQLITQLMPFGCLLDYVREHKDNIGSQYLLNWC |
| | | VQIAKGMNYLEDRRLVHRDLAARNVLVKTPQHVKITDFGLAKLLGAEEKEYHAEGGKVPIKWMALESILHRIY |
| | | THQSDVWSYGVTVWELMTFGSKPYDGIPASEISSILEKGERLPQPPICTIDVYMIMVKCWMIDADSRPKFREL |
| | | IIEFSKMARDPQRYLVIQGDERMHLPSPTDSNFYRALMDEEDMDDVVDADEYLIPQQGFFSSPSTSRTPLLS |
| | | SLSATSNNSTVACIDRNGLQSCPIKEDSFLQRYSSDPTGALTEDSIDDTFLPVPEYINQSVPKRPAGSVQNPV |
| | | YHNQPLNPAPSRDPHYQDPHSTAVGNPEYLNTVQPTCVNSTFDSPAHWAQKGSHQISLDNPDYQQDFFPK |
| | | EAKPNGIFKGSTAENAEYLRVAPQSSEFIGA |
| 85 | Mature human | LEEKKVCQGTSNKLTQLGTFEDHFLSLQRMFNNCEVVLGNLEITYVQRNYDLSFLKTIQEVAGYVLIALNTVE |
| | EGFR isoform 2 | RIPLENLQIIRGNMYYENSYALAVLSNYDANKTGLKELPMRNLQEILHGAVRFSNNPALCNVESIQWRDIVSS |
| | | DFLSNMSMDFQNHLGSCQKCDPSCPNGSCWGAGEENCQKLTKIICAQQCSGRCRGKSPSDCCHNQCAA |
| | | GCTGPRESDCLVCRKFRDEATCKDTCPPLMLYNPTTYQMDVNPEGKYSFGATCVKKCPRNYVVTDHGSCV |
| | | RACGADSYEMEEDGVRKCKKCEGPCRKVCNGIGIGEFKDSLSINATNIKHFKNCTSISGDLHILPVAFRGDSF |
| | | THTPPLDPQELDILKTVKEITGLS |
| 86 | Mature human | LEEKKVCQGTSNKLTQLGTFEDHFLSLQRMFNNCEVVLGNLEITYVQRNYDLSFLKTIQEVAGYVLIALNTVE |
| | EGFR isoform 3 | RIPLENLQIIRGNMYYENSYALAVLSNYDANKTGLKELPMRNLQEILHGAVRFSNNPALCNVESIQWRDIVSS |
| | | DFLSNMSMDFQNHLGSCQKCDPSCPNGSCWGAGEENCQKLTKIICAQQCSGRCRGKSPSDCCHNQCAA |
| | | GCTGPRESDCLVCRKFRDEATCKDTCPPLMLYNPTTYQMDVNPEGKYSFGATCVKKCPRNYVVTDHGSCV |
| | | RACGADSYEMEEDGVRKCKKCEGPCRKVCNGIGIGEFKDSLSINATNIKHFKNCTSISGDLHILPVAFRGDSF |
| | | THTPPLDPQELDILKTVKEITGFLLIQAWPENRTDLHAFENLEIIRGRTKQHGQFSLAVVSLNITSLGLRSLKEIS |
| | | DGDVIISGNKNLCYANTINWKKLFGTSGQKTKIISNRGENSCKATGQVCHALCSPEGCWGPEPRDCVSCRN |
| | | VSRGRECVDKCNLLEGEPREFVENSECIQCHPECLPQAMNITCTGRGPDNCIQCAHYIDGPHCVKTCPAGV |
| | | MGENNTLVWKYADAGHVCHLCHPNCTYGPGNESLKAMLFCLFKLSSCNQSNDGSVSHQSGSPAAQESCL |
| | | GWIPSLLPSEFQLGWGGCSHLHAWPSASVIITASSCH |
| 87 | Mature human | LEEKKVCQGTSNKLTQLGTFEDHFLSLQRMFNNCEVVLGNLEITYVQRNYDLSFLKTIQEVAGYVLIALNTVE |
| | EGFR isoform 4 | RIPLENLQIIRGNMYYENSYALAVLSNYDANKTGLKELPMRNLQEILHGAVRFSNNPALCNVESIQWRDIVSS |
| | | DFLSNMSMDFQNHLGSCQKCDPSCPNGSCWGAGEENCQKLTKIICAQQCSGRCRGKSPSDCCHNQCAA |
| | | GCTGPRESDCLVCRKFRDEATCKDTCPPLMLYNPTTYQMDVNPEGKYSFGATCVKKCPRNYVVTDHGSCV |
| | | RACGADSYEMEEDGVRKCKKCEGPCRKVCNGIGIGEFKDSLSINATNIKHFKNCTSISGDLHILPVAFRGDSF |
| | | THTPPLDPQELDILKTVKEITGFLLIQAWPENRTDLHAFENLEIIRGRTKQHGQFSLAVVSLNITSLGLRSLKEIS |
| | | DGDVIISGNKNLCYANTINWKKLFGTSGQKTKIISNRGENSCKATGQVCHALCSPEGCWGPEPRDCVSCRN |
| | | VSRGRECVDKCNLLEGEPREFVENSECIQCHPECLPQAMNITCTGRGPDNCIQCAHYIDGPHCVKTCPAGV |
| | | MGENNTLVWKYADAGHVCHLCHPNCTYGS |
| 88 | Human EGFR | LEEKKVCQGTSNKLTQLGTFEDHFLSLQRMFNNCEVVLGNLEITYVQRNYDLSFLKTIQEVAGYVLIALNTVE |
| | isoform 1 | RIPLENLQIIRGNMYYENSYALAVLSNYDANKTGLKELPMRNLQEILHGAVRFSNNPALCNVESIQWRDIVSS |
| | extracellular | DFLSNMSMDFQNHLGSCQKCDPSCPNGSCWGAGEENCQKLTKIICAQQCSGRCRGKSPSDCCHNQCAA |
| | region | GCTGPRESDCLVCRKFRDEATCKDTCPPLMLYNPTTYQMDVNPEGKYSFGATCVKKCPRNYVVTDHGSCV |
| | | RACGADSYEMEEDGVRKCKKCEGPCRKVCNGIGIGEFKDSLSINATNIKHFKNCTSISGDLHILPVAFRGDSF |
| | | THTPPLDPQELDILKTVKEITGFLLIQAWPENRTDLHAFENLEIIRGRTKQHGQFSLAVVSLNITSLGLRSLKEIS |
| | | DGDVIISGNKNLCYANTINWKKLFGTSGQKTKIISNRGENSCKATGQVCHALCSPEGCWGPEPRDCVSCRN |
| | | VSRGRECVDKCNLLEGEPREFVENSECIQCHPECLPQAMNITCTGRGPDNCIQCAHYIDGPHCVKTCPAGV |
| | | MGENNTLVWKYADAGHVCHLCHPNCTYGCTGPGLEGCPTNGPKIPS |
| 89 | Human EGFR | IATGMVGALLLLLVVALGIGLFM |
| | isoform 1 | |
| | transmembrane | |
| | domain | |
| 90 | Human EGFR | RRRHIVRKRTLRRLLQERELVEPLTPSGEAPNQALLRILKETEFKKIKVLGSGAFGTVYKGLWIPEGEKVKIPV |
| | isoform 1 | AIKELREATSPKANKEILDEAYVMASVDNPHVCRLLGICLTSTVQLITQLMPFGCLLDYVREHKDNIGSQYLLN |
| | cytoplasmic | WCVQIAKGMNYLEDRRLVHRDLAARNVLVKTPQHVKITDFGLAKLLGAEEKEYHAEGGKVPIKWMALESILH |
| | domain | RIYTHQSDVWSYGVTVWELMTFGSKPYDGIPASEISSILEKGERLPQPPICTIDVYMIMVKCWMIDADSRPKF |
| | | RELIIEFSKMARDPQRYLVIQGDERMHLPSPTDSNFYRALMDEEDMDDVVDADEYLIPQQGFFSSPSTSRTP |
| | | LLSSLSATSNNSTVACIDRNGLQSCPIKEDSFLQRYSSDPTGALTEDSIDDTFLPVPEYINQSVPKRPAGSVQ |
| | | NPVYHNQPLNPAPSRDPHYQDPHSTAVGNPEYLNTVQPTCVNSTFDSPAHWAQKGSHQISLDNPDYQQDF |
| | | FPKEAKPNGIFKGSTAENAEYLRVAPQSSEFIGA |
| 91 | cetuximab VH | QVQLKQSGPGLVQPSQSLSITCTVSGFSLTNYGVHWVRQSPGKGLEWLGVIWSGGNTDYNTPFTSRLSINK |
| | | DNSKSQVFFKMNSLQSNDTAIYYCARALTYYDYEFAYWGQGTLVTVSA |
| 92 | cetuximab HC- | GFSLTNYG |
| | CDR1 | |
| 93 | cetuximab HC- | IWSGGNT |
| | CDR2 | |
| 94 | cetuximab HC- | ARALTYYDYEFAY |
| | CDR3 | |
| 95 | cetuximab VL | DILLTQSPVILSVSPGERVSFSCRASQSIGTNIHWYQQRTNGSPRLLIKYASESISGIPSRFSGSGSGTDFTLSI |
| | | NSVESEDIADYYCQQNNNWPTTFGAGTKLELK |
| 96 | cetuximab LC- | QSIGTN |
| | CDR1 | |
| 97 | cetuximab LC- | YAS |
| | CDR2 | |
| 98 | cetuximab LC- | QQNNNWPTT |
| | CDR3 | |
| 99 | cetuximab HC | QVQLKQSGPGLVQPSQSLSITCTVSGFSLTNYGVHWVRQSPGKGLEWLGVIWSGGNTDYNTPFTSRLSINK |
| | | DNSKSQVFFKMNSLQSNDTAIYYCARALTYYDYEFAYWGQGTLVTVSAASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKSRWQQGNVFSCS |
| | | VMHEALHNHYTQKSLSLSPGK |
| 100 | cetuximab LC | DILLTQSPVILSVSPGERVSFSCRASQSIGTNIHWYQQRTNGSPRLLIKYASESISGIPSRFSGSGSGTDFTLSI |
| | | NSVESEDIADYYCQQNNNWPTTFGAGTKLELKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTHQGLSSPVTKSFNRGEC |

The present disclosure includes the combination of the aspects and preferred features described except where such a combination is clearly impermissible or expressly avoided.

The section headings used herein are for organizational purposes only and are not to be construed as limiting the subject-matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise’, and variations such as ‘comprises’ and ‘comprising’, will be understood to imply the inclusion of a stated integer or step or group of integers or steps but not the exclusion of any other integer or step or group of integers or steps.

It must be noted that, as used in the specification and the appended claims, the singular forms ‘a’, ‘an’, and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about’, it will be understood that the particular value forms another embodiment.

Where a nucleic acid sequence is disclosed or referred to herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

FIGS. 1A and 1B. Graphs showing the effects of treatment of mice having a KYSE-150 cell-derived mouse model of esophageal squamous cell carcinoma with 10D1F (HMBD-001), cetuximab, 10D1F in combination with cetuximab, or vehicle control (PBS). FIG. 1A shows tumor volume over time, for mice in the different treatment groups. FIG. 1B shows bodyweight over time, for mice in the different treatment groups.

FIGS. 2A and 2B. Graphs showing the effects of treatment of mice having a OE21 cell-derived mouse model of esophageal squamous cell carcinoma with 10D1F (HMBD-001), cetuximab, 10D1F in combination with cetuximab, or vehicle control (PBS). FIG. 2A shows tumor volume over time, for mice in the different treatment groups. FIG. 2B shows bodyweight over time, for mice in the different treatment groups.

FIGS. 3A and 3B. Graphs showing the effects of treatment of mice having a LIM1215 cell-derived mouse model of colon adenocarcinoma with 10D1F (HMBD-001), cetuximab, 10D1F in combination with cetuximab, or vehicle control (PBS). FIG. 3A shows tumor volume over time, for mice in the different treatment groups. FIG. 3B shows bodyweight over time, for mice in the different treatment groups.

FIGS. 4A and 4B. Graphs showing the effects of treatment of mice having a CAL-27 cell-derived mouse model of tongue squamous cell carcinoma with 10D1F (HMBD-001), cetuximab, 10D1F in combination with cetuximab, or vehicle control (PBS). FIG. 4A shows tumor volume over time, for mice in the different treatment groups. FIG. 4B shows bodyweight over time, for mice in the different treatment groups.

FIGS. 5A and 5B. Graphs showing the effects of treatment of mice having a FaDu cell-derived mouse model of hypopharyngeal squamous cell carcinoma with 10D1F (HMBD-001), cetuximab, 10D1F in combination with cetuximab, or vehicle control (PBS). FIG. 5A shows tumor volume over time, for mice in the different treatment groups. FIG. 5B shows bodyweight over time, for mice in the different treatment groups.

FIGS. 6A and 6B. Graphs showing the effects of treatment of mice having a BxPC-3 cell-derived mouse model of pancreatic ductal adenocarcinoma with 10D1F (HMBD-001), cetuximab, 10D1F in combination with cetuximab, or vehicle control (PBS). FIG. 6A shows tumor volume over time, for mice in the different treatment groups. FIG. 6B shows bodyweight over time, for mice in the different treatment groups.

FIGS. 7A and 7B. Graphs showing the effects of treatment of mice having a human patient-derived mouse model (CTG-2552) of lung squamous cell carcinoma with 10D1F (HMBD-001), cetuximab, 10D1F in combination with cetuximab, or isotype control. FIG. 7A shows tumor volume over time, for mice in the different treatment groups. FIG. 7B shows bodyweight over time, for mice in the different treatment groups.

FIGS. 8A, 8B and 8C. Graphs showing the effects of treatment of mice having a a cell line-derived xenograft (CDX) model of lung squamous cell carcinoma with 10D1F (HMBD-001), cetuximab (5 mg/kg and 10 mg/kg), docetaxel, 10D1F in combination with cetuximab (5 mg/kg and 10 mg/kg), 10D1F in combination with docetaxel, cetuximab (5 mg/kg and 10 mg/kg) in combination with docetaxel, 10D1F in combination with cetuximab (5 mg/kg and 10 mg/kg) and docetaxel, or vehicle control (PBS). FIG. 8A shows tumor volume over time, for mice in the different treatment groups (5 mg/kg cetuximab in the treatment groups including cetuximab). FIG. 8B shows tumor volume over time, for mice in the different treatment groups (10 mg/kg cetuximab in the treatment groups including cetuximab). FIG. 8C shows bodyweight over time, for mice in the different treatment groups.

### EXAMPLES

### Example 1: Characterization of 10D1F in WO 2019/185878 A1 and WO 2021/048274 A1

The HER3-binding antibody clone designated 10D1F is described in WO 2019/185878 A1 (incorporated by reference in its entirety).

10D1F comprises the heavy chain variable region shown in SEQ ID NO:36 of WO 2019/185878 A1 (=SEQ ID NO:33 of the present disclosure), and the light chain variable region shown in SEQ ID NO:83 of WO 2019/185878 A1 (=SEQ ID NO:58 of the present disclosure). 10D1F is also referred to in WO 2019/185878 A1 as ‘10D1_c89’, and is sometimes referred to herein as HMBD-001.

Example 2.2 of WO 2019/185878 A1 describes a molecule (molecule [16]) comprising the VH and VL regions of 10D1F in human IgG1/Vκ format (10D1F hlgG1), formed of SEQ ID NO:206 of WO 2019/185878 A1 (=SEQ ID NO:75 of the present disclosure) and SEQ ID NO:207 of WO 2019/185878 A1 (=SEQ ID NO:76 of the present disclosure).

Examples 8.1 to 8.3 and FIGS. 42 to 46 of WO 2019/185878 A1 show that 10D1F hlgG1 binds to human HER3 with high affinity and specificity (displaying no cross-reactivity with other human EGFR family members), while retaining high-affinity binding to cyno, mouse and rat HER3.

Example 8.6 and FIGS. 49A and 49B of WO 2019/185878 A1 demonstrate that 10D1F hlgG1 binds to HER3 in a ligand (NRG)-independent fashion, and through a topologically distant epitope of HER3 to the epitope bound by anti-HER3 antibodies M-05-74 and M-08-11. Example 8.10 and FIG. 78 of WO 2021/048274 A1 demonstrate that 10D1F hlgG1 binds to human HER3 with subpicomolar affinity in the presence or absence of human NRG1.

WO 2021/048274 A1 at Example 3.5 also discloses that antibody clone 10D1 and 10D1-derived clones (including 10D1F) bind to human HER3 in the region corresponding to positions 218 to 235 of SEQ ID NO: 1 (i.e. SEQ ID NO:77 of the present disclosure), and that within this region, two consensus binding site motifs were identified (shown in SEQ ID NOs: 78 and 79 of the present disclosure).

Example 4.1 and FIG. 65, and Example 8.7 and FIG. 52 of WO 2019/185878 A1 demonstrate that 10D1F hlgG1 is highly potent at inhibiting interaction between HER3 and HER2, and does so in a dose-dependent manner. Example 8.7 and FIG. 53 of WO 2019/185878 A1 show that 10D1F hlgG1 inhibits interaction between HER3 and EGFR in a dose-dependent fashion.

Example 8.8 and FIG. 54 of WO 2019/185878 A1 show that 10D1F hlgG1 induces ADCC activity against HER3 overexpressing cells in a dose-dependent manner.

Example 8.9 and FIGS. 55, 63 and 64 of WO 2019/185878 A1 demonstrate that 10D1F hlgG1 inhibits HER3-mediated signaling in cells of HER3-expressing cancer cell lines in vitro.

Example 11 and FIG. 71 of WO 2019/185878 A1 show that 10D1F hlgG1 also inhibits HER3-mediated signaling in HER3-expressing human cancer cell line-derived xenograft tumors in vivo. Example 14 and FIG. 79 of WO 2021/048274 A1 demonstrate that 10D1F is extremely potent at inhibiting growth of xenograft tumors derived from a human cancer cell line harbouring an NRG gene fusion.

Examples 9.3, 9.4 and FIGS. 59, 60, 61, 62, 74 and 77 of WO 2019/185878 A1 demonstrate that 10D1F potently inhibits the growth of cancer cells in vitro, and also potently inhibits growth of human cancer cell line-derived xenograft tumors in vivo. Example 10 and FIGS. 67 and 68 of WO 2019/185878 A1 show that 10D1F hlgG1 inhibits in vitro proliferation of thyroid cancer cell lines harbouring the V600E BRAF mutation.

Example 12 and FIGS. 72 and 73 of WO 2019/185878 A1 show that 10D1F hlgG1 is not substantially internalized by HER3-expressing cells.

Example 13 and FIGS. 75 and 76 of WO 2019/185878 A1 demonstrate the utility of 10D1F hlgG1 to be employed for the detection of HER3.

Example 8.4 and FIG. 47A of WO 2019/185878 A1 show that 10D1F hlgG1 is thermostable, having a melting temperature of 70.0° C. as determined by Differential Scanning Fluorimetry.

Example 9.1, 9.2 and FIGS. 56, 57, 58 and 69, 70 of WO 2019/185878 A1 evidence that 10D1F hlgG1 has favorable pharmacological and toxicological profiles.

### Example 2: Evaluation of Therapeutic Efficacy of Combination Treatment Using 10D1F and Cetuximab

The therapeutic efficacy of the combination of 10D1F hlgG1 (i.e. the antibody formed by the polypeptide consisting of SEQ ID NO:75 and the polypeptide consisting of SEQ ID NO:76) and cetuximab (i.e. the antibody formed by the polypeptide consisting of SEQ ID NO:99 and the polypeptide consisting of SEQ ID NO: 100) was investigated in various different cancers in vivo, in human cell and/or patient-derived xenograft models.

Mice approximately 6-8 weeks old were housed under specific pathogen-free conditions and treated in compliance with the Institutional Animal Care and Use Committee (IACUC) guidelines. Human cancer cell-derived tumors were established by admixture of cells with equal volume of Matrigel (Corning, USA), and subcutaneous implantation into the right or left flanks of mice, as indicated. Treatment was initiated when tumors reached approximately 100 to 300 mm³.

### ESCC CDX (1):

Model: Human cell line-derived model of esophageal squamous cell carcinoma Cell line: KYSE-150 (CVCL_1348). KYSE-150 cells display high expression of NRG1 and HER3 (Meetze et al., Clinical Cancer Research (2015) 21 (5): 1106-1114).

Model established by: injection of 1×10⁷ cells into right flanks of NOD/SCID mice.

### Treatment Groups:

Vehicle control (phosphate buffered saline; IP injection, weekly; n=10).
HMBD-001 (IP injection of 20 mg/kg bodyweight, weekly; n=10).
cetuximab (IP injection of 10 mg/kg bodyweight, weekly; n=10).
HMBD-001+cetuximab (IP injection of 20 mg/kg bodyweight of HMBD-001+10 mg/kg
bodyweight of cetuximab, weekly; n=10).

### ESCC CDX (2):

Model: Human cell line-derived model of esophageal squamous cell carcinoma
Cell line: OE21 (CVCL_2661). OE21 cells express high levels of EGFR dimers (Fichter et al., Int J Cancer (2014) 135 (7): 1517-1530).
Model established by: injection of 1×10⁶ cells into right flanks of NOD/SCID mice.
Treatment groups:
   Vehicle control (phosphate buffered saline; IP injection, twice weekly; n=10).
   HMBD-001 (IP injection of 20 mg/kg bodyweight, twice weekly; n=10).
   cetuximab (IP injection of 10 mg/kg bodyweight, twice weekly; n=10).
   HMBD-001+cetuximab (IP injection of 20 mg/kg bodyweight of HMBD-001+10 mg/kg
   bodyweight of cetuximab, twice weekly; n=10).

### CRC CDX:

Model: Human cell line-derived model of colon adenocarcinoma
Cell line: LIM1215 (CVCL_2574). LIM1215 cells express normal levels of EGFR, but are sensitive to cetuximab (Misale et al., Nature, (2012) 486 (7404): 532-536).
Model established by: injection of 2×10⁶ cells into right flanks of Nude mice.

### Treatment Groups:

Vehicle control (phosphate buffered saline; IP injection, twice weekly; n=10).
HMBD-001 (IP injection of 20 mg/kg bodyweight, twice weekly; n=11).
cetuximab (IP injection of 10 mg/kg bodyweight, twice weekly; n=12).
HMBD-001+cetuximab (IP injection of 20 mg/kg bodyweight of HMBD-001+10 mg/kg
bodyweight of cetuximab, twice weekly; n=12).

### HNSCC CDX (1):

Model: Human cell line-derived model of tongue squamous cell carcinoma
Cell line: CAL-27 (CVCL_1107). CAL-27 cells overexpress EGFR (Licitra et al., Ann Oncol (2011) 22(8):1886-1893).
Model established by: injection of 5×10⁶ cells into right flanks of NOD SCID gamma (NSG) mice.
Treatment groups:
   Vehicle control (phosphate buffered saline; IP injection, twice weekly; n=8).
   HMBD-001 (IP injection of 20 mg/kg bodyweight, twice weekly; n=8).
   cetuximab (IP injection of 10 mg/kg bodyweight, twice weekly; n=8).
   HMBD-001+cetuximab (IP injection of 20 mg/kg bodyweight of HMBD-001+10 mg/kg
   bodyweight of cetuximab, twice weekly; n=8).

### HNSCC CDX (2):

Model: Human cell line-derived model of hypopharyngeal squamous cell carcinoma
Cell line: FaDu (CVCL_1218). FaDu cells display high expression of EGFR and NRG1 (Xiao et al., Mol Cancer Ther (2016) 15 (4): 689-701).
Model established by: injection of 1×10⁶ cells into right flanks of NCr Nude mice.

### Treatment Groups:

Vehicle control (phosphate buffered saline; IP injection, twice weekly; n=10).
HMBD-001 (IP injection of 20 mg/kg bodyweight, twice weekly; n=10).
cetuximab (IP injection of 10 mg/kg bodyweight, twice weekly; n=10).
HMBD-001+cetuximab (IP injection of 20 mg/kg bodyweight of HMBD-001+10 mg/kg
bodyweight of cetuximab, twice weekly; n=10).

### PDAC CDX:

Model: Human cell line-derived model of pancreatic ductal adenocarcinoma
Cell line: BxPC-3 (CVCL_0186).
Model established by: injection of 5×10⁶ cells into right flanks of NOD/SCID mice.

### Treatment Groups:

Vehicle control (phosphate buffered saline; IP injection, twice weekly; n=11).
HMBD-001 (IP injection of 20 mg/kg bodyweight, twice weekly; n=11).
cetuximab (IP injection of 10 mg/kg bodyweight, twice weekly; n=11).
HMBD-001+cetuximab (IP injection of 20 mg/kg bodyweight of HMBD-001+10 mg/kg
bodyweight of cetuximab, twice weekly; n=11).

### LUSC PDX:

Model: Human patient-derived model (CTG-2552) of lung squamous cell carcinoma
Model established by: implantation of 3-4 tumor fragments into left flanks of Athymic, Foxn 1nu Nude mice.

### Treatment Groups:

Isotype control (Human IgG1 kappa isotype control; IP injection of 20 mg/kg bodyweight, weekly; n=10).
HMBD-001 (IP injection of 20 mg/kg bodyweight, weekly; n=10).
cetuximab (IP injection of 10 mg/kg bodyweight, weekly; n=10).
HMBD-001+cetuximab (IP injection of 20 mg/kg bodyweight of HMBD-001+10 mg/kg
bodyweight of cetuximab, weekly; n=10).

Tumor volume was measured 2 times a week using a digital caliper, and calculated using the formula [L×W×W)/2]. Study end point was reached once the mean tumor volume of the control group reached 1500 mm³. Bodyweights of the mice were also monitored.

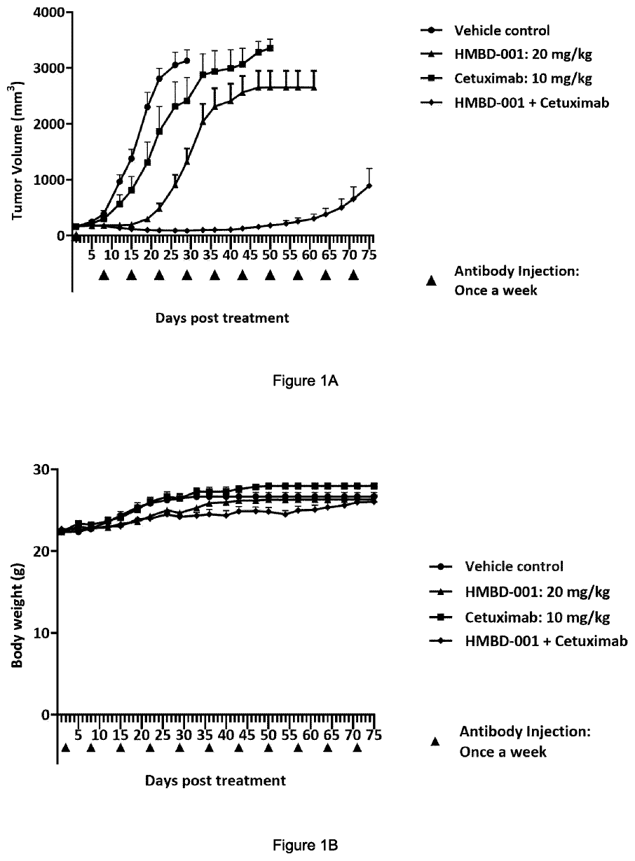

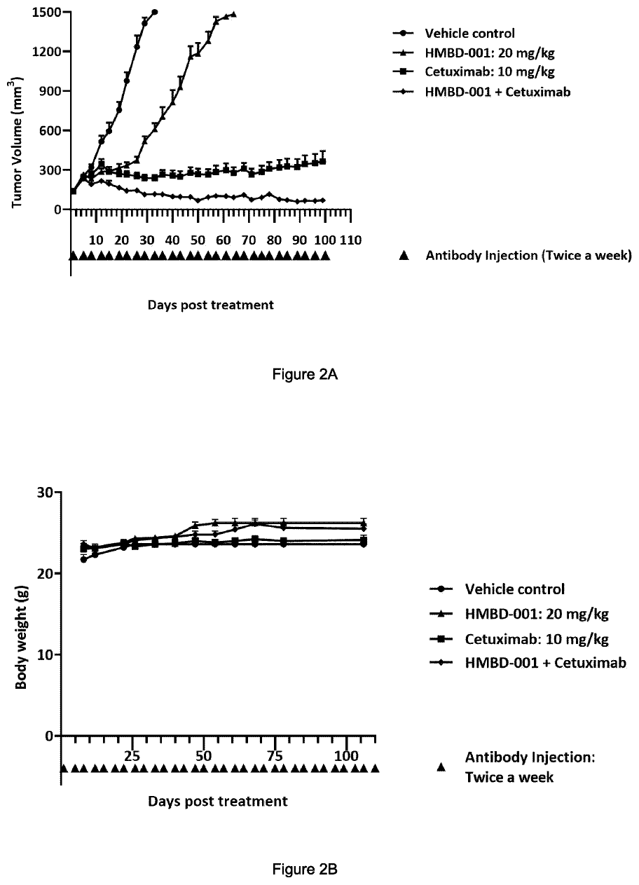

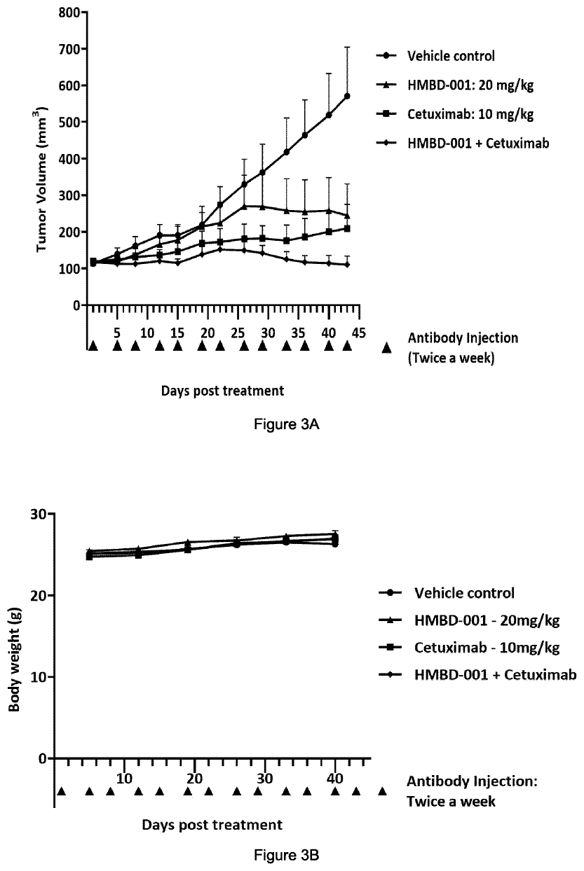

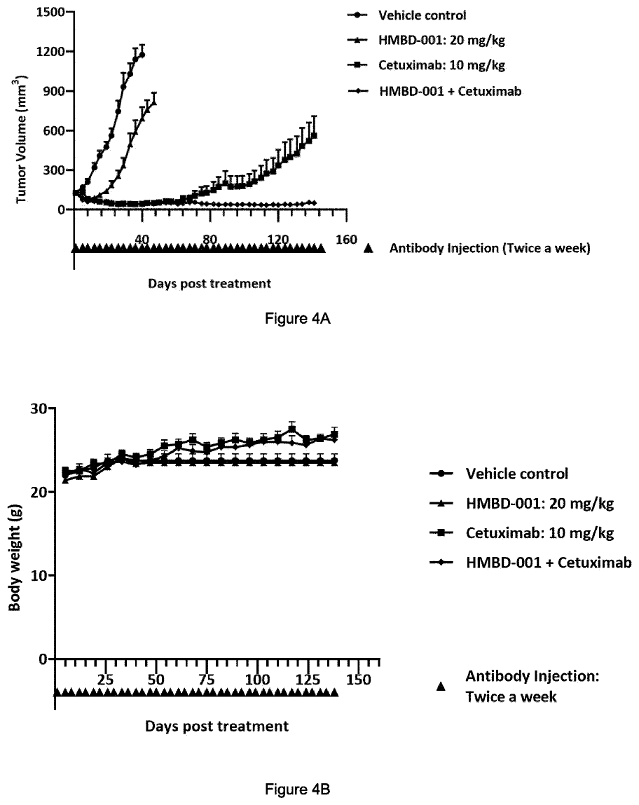

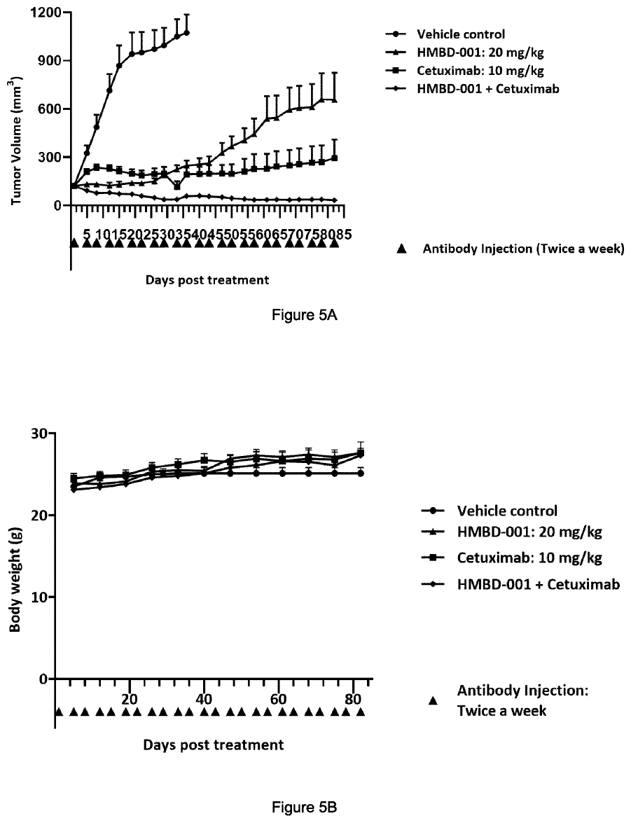

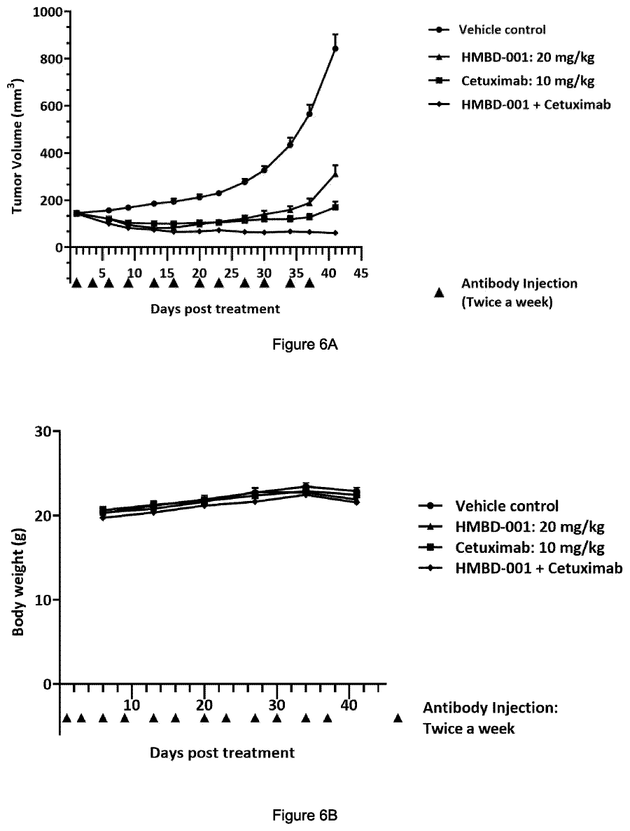

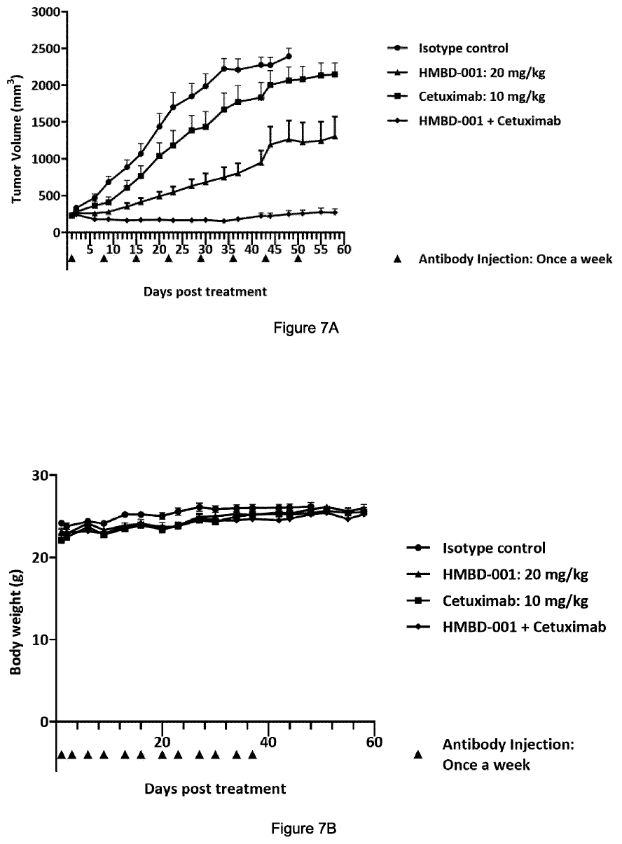

The results of the experiments are shown in FIGS. 1 to 7.

Combination treatment with HMBD-001 and cetuximab achieved greater tumor growth inhibition than treatment with either agent alone in the ESCC, CRC, HNSCC and PDAC CDX models, and in the LUSC PDX model.

Discussion—Anti-HER3 antibody, 10D1F, in combination with an EGFR inhibitor effectively inhibits tumor growth in biomarker selected pre-clinical models of squamous cell carcinomas.

Squamous cell carcinomas (SCC) originate from stratified squamous epithelium in diverse anatomical locations that share common histological features and genetic mutations. Non-melanoma skin cancer, head and neck cancer (HNSCC), esophageal cancer (ESCC), and non-small cell lung cancer (sqNSCLC) make up the majority of SCC cases and together represent the largest subtype of cancer. There is a significant unmet need for effective treatment options post progression of standard-of-care therapies. Alterations in receptor tyrosine kinase signaling pathways are commonly found in SCCs, e.g. EGFR is expressed in 90% of HNSCC, 76% of ESCC and 82% of sqNSCLC. However, the combination of EGFR targeting antibody cetuximab with chemotherapy confers limited clinical benefit and even initially sensitive tumors often develop resistance. Studies in SCC have implicated HER3, a key dimerization partner of HER family members, as one likely cause of treatment failure, through HER3 heterodimer activation of PI3K/AKT and MAPK/ERK pathways. In this study, we investigated the potential of combining HER3 and EGFR inhibitors to improve efficacy and overcome resistance.

Previous attempts to target HER3 have shown limited clinical efficacy due to suboptimal HER3 inhibition, especially the lack of efficacy in ligand-independent activation mechanisms, as well as a lack of biomarker stratification for patients where HER3 inhibition would confer benefit. 10D1F is a clinical-stage anti-HER3 antibody, that is being evaluated in a first in human, open-label, multi-center, dose escalation and expansion Phase I/IIa trial in patients with HER3 expressing advanced solid tumors (NCT05057013). 10D1F was rationally developed to uniquely block the HER3 heterodimerization interface to potently inhibit all HER3 dimer formation, including ligand-dependent and independent HER3 dimerization.

Here, we show that dual blockade of EGFR and HER3 using cetuximab and 10D1F respectively, robustly inhibits tumor growth and is superior to either monotherapy. In multiple cell and patient derived SCC xenograft models, including models of HNSCC, ESCC and sqNSCLC, selected based on a novel gene signature that is robustly predictive of response, 10D1F in combination with cetuximab demonstrated up to 100% tumor growth inhibition and the abrogation of PI3K signaling. This combination was well tolerated with no relapse observed, even after a prolonged period of over 100 days in selected models.

### Example 3: Evaluation of Therapeutic Efficacy of Combination Treatment Using 10D1F. Cetuximab, and Docetaxel

The therapeutic efficacy of the combination of 10D1F hlgG1 (i.e. the antibody formed by the polypeptide consisting of SEQ ID NO:75 and the polypeptide consisting of SEQ ID NO:76), cetuximab (i.e. the antibody formed by the polypeptide consisting of SEQ ID NO:99 and the polypeptide consisting of SEQ ID NO: 100), and docetaxel was investigated in lung squamous cell carcinoma (LUSC) in vivo, in a cell line-derived xenograft (CDX) model. Cell line: HARA (KCC-C1). HARA cells display high expression of parathyroid hormone-related protein (PTHrP) and interleukin 1 (IL-1) (Ichinose et al., Cancer Letters (1993) 74 (1-2): 119-124).

NOD/SCID mice approximately 6-8 weeks old were housed under specific pathogen-free conditions and treated in compliance with the Institutional Animal Care and Use Committee (IACUC) guidelines. Human cell line-derived tumors were established by admixture of cells with equal volume of Matrigel (Corning, USA), and subcutaneous implantation of 2×10⁶ cells into the right flanks of mice. Treatment was initiated when tumors reached approximately 150 to 200 mm³.

### Treatment Groups:

Vehicle control (phosphate buffered saline; IP injection, weekly; n=8).
HMBD-001 (IP injection of 20 mg/kg bodyweight, weekly; n=8).
Cetuximab (IP injection of 5 mg/kg bodyweight, weekly; n=8).
Cetuximab (IP injection of 10 mg/kg bodyweight, weekly; n=8).
Docetaxel (IP injection of 10 mg/kg bodyweight, weekly; n=8).
HMBD-001+cetuximab (IP injection of 20 mg/kg bodyweight of HMBD-001+5 mg/kg bodyweight of cetuximab, weekly; n=8).
HMBD-001+cetuximab (IP injection of 20 mg/kg bodyweight of HMBD-001+10 mg/kg bodyweight of cetuximab, weekly; n=8).
HMBD-001+docetaxel (IP injection of 20 mg/kg bodyweight of HMBD-001+10 mg/kg bodyweight of docetaxel, weekly; n=8).
Cetuximab+docetaxel (IP injection of 5 mg/kg bodyweight of cetuximab+10 mg/kg bodyweight of docetaxel, weekly; n=8).
Cetuximab+docetaxel (IP injection of 10 mg/kg bodyweight of cetuximab+10 mg/kg bodyweight of docetaxel, weekly; n=8).
HMBD-001+cetuximab+docetaxel (IP injection of 20 mg/kg bodyweight of HMBD-001+5 mg/kg bodyweight of cetuximab+10 mg/kg bodyweight of docetaxel, weekly; n=8).
HMBD-001+cetuximab+docetaxel (IP injection of 20 mg/kg bodyweight of HMBD-001+10 mg/kg bodyweight of cetuximab+10 mg/kg bodyweight of docetaxel, weekly; n=8).

Tumor volume was measured 2 times a week using a digital caliper, and calculated using the formula [L×W×W)/2]. Bodyweights of the mice were also monitored.

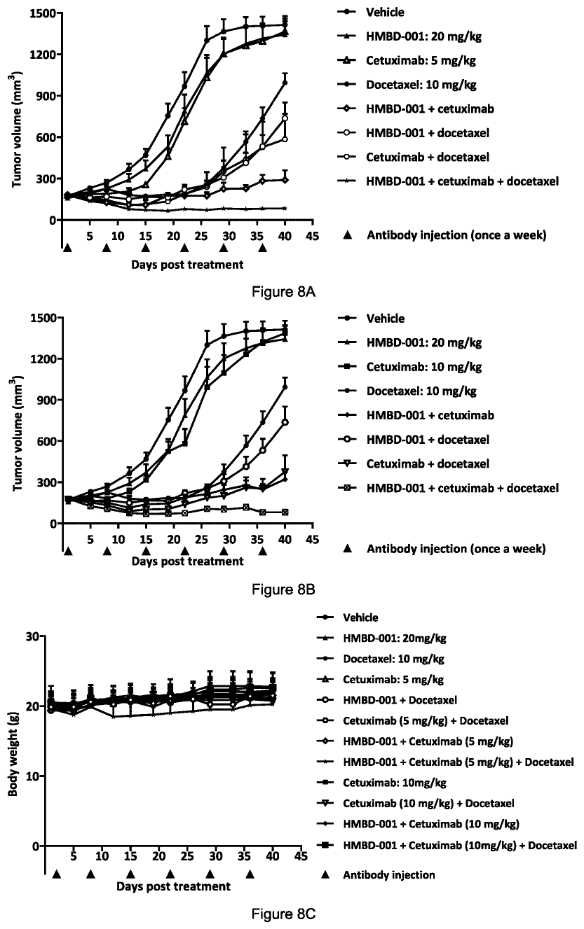

The results of the experiment are shown in FIG. 8. Specifically, FIGS. 8A and 8B show tumor volume over time, for mice in the different treatment groups (8A: 5 mg/kg cetuximab in the treatment groups including cetuximab; 8B: 10 mg/kg cetuximab in the treatment groups including cetuximab). FIG. 8C shows bodyweight over time, for mice in the different treatment groups.

Combination treatment with HMBD-001, cetuximab and docetaxel achieved greater tumor growth inhibition than treatment with any of the agents alone, and any combination of two of the agents, in the LUSC CDX model. In fact, at both dose levels of cetuximab (5 mg/kg and 10 mg/kg), combination treatment with HMBD-001, cetuximab and docetaxel achieved an overall reduction in tumor volume at 40 days post initial treatment, which was not achieved by any other treatment group. This combination was well tolerated as indicated by the bodyweight of the mice over the treatment period (FIG. 8C).

Discussion—Anti-HER3 antibody, 10D1F, in combination with an EGFR inhibitor and a taxane (docetaxel) effectively inhibits tumor growth in a cell line derived xenograft model of lung squamous cell carcinoma (LUSC).

In this study, we investigated the potential of combining HER3 and EGFR inhibitors with a taxane (docetaxel) to improve efficacy.

Here, we show that the addition of docetaxel to a dual blockade of EGFR and HER3 using cetuximab and 10D1F respectively, robustly inhibits tumor growth and is superior to monotherapy with any of the three agents individually, and any combination therapy using two of the three agents. In fact, at both dose levels of cetuximab (5 mg/kg and 10 mg/kg), combination treatment with HMBD-001, cetuximab and docetaxel achieved an overall reduction in tumor volume at 40 days post initial treatment, which was not achieved by any other treatment group. In a cell line-derived LUSC xenograft model, 10D1F in combination with cetuximab and docetaxel demonstrated >100% tumor growth inhibition and reduction in overall tumor volume at 40 days after initial treatment. This combination was well tolerated with no relapse observed.

1. An antigen-binding molecule that binds to HER3 for use in a method of treating or preventing a cancer, wherein the method comprises administering an antigen-binding molecule that binds to EGFR, and wherein the antigen-binding molecule that binds to HER3 binds to the region of HER3 shown in SEQ ID NO: 77.

2. Use of an antigen-binding molecule that binds to HER3 in the manufacture of a medicament for use in a method of treating or preventing a cancer, wherein the method comprises administering an antigen-binding molecule that binds to EGFR, and wherein the antigen-binding molecule that binds to HER3 binds to the region of HER3 shown in SEQ ID NO:77.

3. A method of treating or preventing a cancer, comprising administering to a subject a therapeutically- or prophylactically-effective amount of (i) an antigen-binding molecule that binds to HER3 and (ii) an antigen-binding molecule that binds to EGFR; wherein the antigen-binding molecule that binds to HER3 binds to the region of HER3 shown in SEQ ID NO:77.

4. The antigen-binding molecule for use, the use, or the method according to any one of claims 1 to 3, wherein the method of treating or preventing a cancer further comprises administering docetaxel.

5. A pharmaceutical combination, comprising an antigen-binding molecule that binds to HER3 and an antigen-binding molecule that binds to EGFR; wherein the antigen-binding molecule that binds to HER3 binds to the region of HER3 shown in SEQ ID NO:77.

6. A pharmaceutical combination according to claim 5, for use in a method of treating or preventing a cancer.

7. Use of a pharmaceutical combination according to claim 5, in the manufacture of a medicament for use in a method of treating or preventing a cancer.

8. A method of treating or preventing a cancer, comprising administering to a subject a therapeutically- or prophylactically-effective amount of a pharmaceutical combination according to claim 5.

9. The pharmaceutical combination for use, the use, or the method according to any one of claims 6-8, wherein the method of treating or preventing a cancer further comprises administering docetaxel.

10. The antigen-binding molecule for use, the use, or the method according to any one of claims 1 to 9, wherein the antigen-binding molecule that binds to HER3 comprises:
(i) a heavy chain variable (VH) region incorporating the following CDRs:
HC-CDR1 having the amino acid sequence of SEQ ID NO:40
HC-CDR2 having the amino acid sequence of SEQ ID NO:43
HC-CDR3 having the amino acid sequence of SEQ ID NO:48; and
(ii) a light chain variable (VL) region incorporating the following CDRs:
LC-CDR1 having the amino acid sequence of SEQ ID NO:66
LC-CDR2 having the amino acid sequence of SEQ ID NO:69
LC-CDR3 having the amino acid sequence of SEQ ID NO:74.

11. The antigen-binding molecule for use, the use, or the method according to any one of claims 1 to 10, wherein the antigen-binding molecule that binds to HER3 comprises:
(i) a VH region incorporating the following CDRs:
HC-CDR1 having the amino acid sequence of SEQ ID NO:38
HC-CDR2 having the amino acid sequence of SEQ ID NO:42
HC-CDR3 having the amino acid sequence of SEQ ID NO:45; and
(ii) a VL region incorporating the following CDRs:
LC-CDR1 having the amino acid sequence of SEQ ID NO:63
LC-CDR2 having the amino acid sequence of SEQ ID NO:67
LC-CDR3 having the amino acid sequence of SEQ ID NO:70.

12. The antigen-binding molecule for use, the use, or the method according to any one of claims 1 to 11, wherein the antigen-binding molecule that binds to HER3 comprises:
a VH region comprising an amino acid sequence having at least 70% sequence identity to the amino acid sequence of SEQ ID NO:33; and
a VL region comprising an amino acid sequence having at least 70% sequence identity to the amino acid sequence of SEQ ID NO:58.

13. The antigen-binding molecule for use, the use, or the method according to any one of claims 1 to 12, wherein the antigen-binding molecule that binds to HER3 comprises:
a polypeptide comprising, or consisting of, an amino acid sequence having at least 70% sequence identity to the amino acid sequence of SEQ ID NO:75; and
a polypeptide comprising, or consisting of, an amino acid sequence having at least 70% sequence identity to the amino acid sequence of SEQ ID NO:76.

14. The antigen-binding molecule for use, the use, or the method according to any one of claims 1 to 13, wherein the antigen-binding molecule that binds to EGFR comprises:
(i) a heavy chain variable (VH) region incorporating the following CDRs:
HC-CDR1 having the amino acid sequence of SEQ ID NO:92
HC-CDR2 having the amino acid sequence of SEQ ID NO:93
HC-CDR3 having the amino acid sequence of SEQ ID NO:94; and
(ii) a light chain variable (VL) region incorporating the following CDRs:
LC-CDR1 having the amino acid sequence of SEQ ID NO:96
LC-CDR2 having the amino acid sequence of SEQ ID NO:97
LC-CDR3 having the amino acid sequence of SEQ ID NO:98.

15. The antigen-binding molecule for use, the use, or the method according to any one of claims 1 to 14, wherein the antigen-binding molecule that binds to EGFR comprises:
a VH region comprising an amino acid sequence having at least 70% sequence identity to the amino acid sequence of SEQ ID NO:91; and
a VL region comprising an amino acid sequence having at least 70% sequence identity to the amino acid sequence of SEQ ID NO:95.

16. The antigen-binding molecule for use, the use, or the method according to any one of claims 1 to 15, wherein the antigen-binding molecule that binds to EGFR comprises:
a polypeptide comprising, or consisting of, an amino acid sequence having at least 70% sequence identity to the amino acid sequence of SEQ ID NO:99; and
a polypeptide comprising, or consisting of, an amino acid sequence having at least 70% sequence identity to the amino acid sequence of SEQ ID NO: 100.

17. The antigen-binding molecule for use, the use, or the method according to any one of claims 1 to 16, wherein the cancer is selected from: a cancer comprising cells expressing/overexpressing an EGFR family member, a cancer comprising cells expressing/overexpressing HER3, a cancer comprising cells expressing/overexpressing EGFR, a cancer comprising cells expressing/overexpressing HER3 and EGFR, a cancer comprising cells having a mutation resulting in increased expression of a ligand for HER3, a cancer comprising cells having a mutation resulting in increased expression of a ligand for EGFR, a cancer comprising cells having an NRG gene fusion, a solid tumor, a hematological cancer, a squamous cell cancer, an EGFR-amplified squamous cell carcinoma, breast cancer, breast carcinoma, breast invasive carcinoma, ductal carcinoma, metastatic breast cancer, triple-negative breast cancer, HER2-positive breast cancer, gastric cancer, gastric carcinoma, gastric adenocarcinoma, gastrointestinal adenocarcinoma, colorectal cancer, metastatic colorectal cancer, colon cancer, colorectal carcinoma, colorectal adenocarcinoma, colon adenocarcinoma, head and neck cancer, head and neck squamous cell carcinoma, lung cancer, non-small cell lung cancer, lung adenocarcinoma, invasive mucinous lung adenocarcinoma, lung squamous cell carcinoma, ovarian cancer, ovarian carcinoma, ovarian serous adenocarcinoma, ovarian serous cystadenocarcinoma, fallopian tube cancer, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, oral cavity cancer, oropharyngeal cancer, esophageal cancer, esophageal squamous cell carcinoma, esophageal adenocarcinoma, liver cancer, hepatocellular carcinoma, cholangiocarcinoma, gallbladder cancer, uterine cancer, endometrial cancer, uterine corpus endometrial carcinoma, uterine carcinosarcoma, thyroid cancer, thyroid cancer, thyroid carcinoma, pheochromocytoma, paraganglioma, bladder cancer, bladder urothelial carcinoma, prostate cancer, prostate adenocarcinoma, retinoblastoma, sarcoma, soft tissue sarcoma, peritoneal cancer, thymoma, neuroendocrine tumor, neuroendocrine tumor of the nasopharynx, squamous cell carcinoma of the skin, astrocytoma, low grade astrocytoma, high grade astrocytoma, anaplastic astrocytoma and glioblastoma multiforme.

18. The antigen-binding molecule for use, the use, or the method according to any one of claims 1 to 17, wherein the cancer is selected from: a cancer comprising cells expressing/overexpressing HER3, a cancer comprising cells expressing/overexpressing EGFR, a cancer comprising cells expressing/overexpressing HER3 and EGFR, a squamous cell cancer, an EGFR-amplified squamous cell carcinoma, esophageal cancer, esophageal squamous cell carcinoma, head and neck cancer, head and neck squamous cell carcinoma, colorectal cancer, metastatic colorectal cancer, colon adenocarcinoma, pancreatic cancer, pancreatic adenocarcinoma, pancreatic ductal adenocarcinoma, lung cancer and lung squamous cell carcinoma.


SEQUENCE LISTING (100 sequences)

SEQ ID NO: 1 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - protein, 183 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTGQFPMVPSGLTPQPAQDWYLLDDDPRLLTLSASSKVPVTLAAV

SEQ ID NO: 3 - protein, 331 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 4 - protein, 1283 aa, Homo sapiens
MGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 5 - protein, 699 aa, Homo sapiens
MALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 6 - protein, 1323 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 7 - protein, 164 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTGQFPMVPSGLTPQPAQDWYLLDDDPRLLTLSASSKVPVTLAAV

SEQ ID NO: 8 - protein, 312 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 9 - protein, 624 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLT

SEQ ID NO: 10 - protein, 21 aa, Homo sapiens
MALTVIAGLVVIFMMLGGTFL

SEQ ID NO: 11 - protein, 678 aa, Homo sapiens
YWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 12 - protein, 44 aa, Homo sapiens
YWRGRRIQNKRAMRRYLERGESIEPLDPSEKANKVLARIFKETE

SEQ ID NO: 13 - protein, 258 aa, Homo sapiens
LRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFT

SEQ ID NO: 14 - protein, 376 aa, Homo sapiens
RMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 15 - protein, 164 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSC

SEQ ID NO: 16 - protein, 146 aa, Homo sapiens
PPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPK

SEQ ID NO: 17 - protein, 166 aa, Homo sapiens
ACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVA

SEQ ID NO: 18 - protein, 148 aa, Homo sapiens
EGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLT

SEQ ID NO: 19 - protein, 17 aa, Homo sapiens
QPLVYNKLTFQLEPNPH

SEQ ID NO: 20 - protein, 1283 aa, Macaca mulatta
MGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWKDIVRDQDAEIVVKDNGRSCPLCHEVCKGRCWGPGPEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMYNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKIIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGEAFQESAVSGSSEWCPRPVSLHPMPRGCLASESSEGHVTGSEAELQEKVSTCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPRPPRPSSLEELGYEYMDVGSDLSASLGSTQSCPLHPVPVMPTAGTTPDEDYEYMNRQRGGSGPGGDYAAMGACPASEQGYEEMRAFQGPGHQAPHVHYAHLKTLRSLEATDSAFDNPDYWHSRLFPKANAQRT

SEQ ID NO: 21 - protein, 120 aa, synthetic construct
DVQLQESGPDLVKPSQSLSLTCTVTGYSITSGYSWHWIRQFPGNKLEWMGSIHYSGGTNYNPSLKSRISITRDTSKNQFFLQLNSVTTEDTATYFCARMTTAPRYPFDYWGQGTTLTVSS

SEQ ID NO: 22 - protein, 120 aa, synthetic construct
DVQLQEWGAGLLKPSETLSLTCAVTGYSITSGYSWHWIRQFPGNGLEWIGSIHYSGGTNYNPTLKSRITISRDTSKNQFSLKLSSVTAADTAVYFCARMTTAPRYPFDYWGQGTLVTVSS

SEQ ID NO: 23 - protein, 120 aa, synthetic construct
DVQLQEWGAGLLKPSETLSLTCAVTGYSITSGYSWHWIRQFPGNGLEWIGSIHYSGGTNYNPSLKSRITISRDTSKNQFSLKLSSVTAADTAVYFCARMTTAPRYPFDYWGQGTLVTVSS

SEQ ID NO: 24 - protein, 120 aa, synthetic construct; identical to SEQ ID NO: 23

SEQ ID NO: 25 - protein, 120 aa, synthetic construct; identical to SEQ ID NO: 23

SEQ ID NO: 26 - protein, 120 aa, synthetic construct
DVQLQEWGAGLLKPSETLSLTCAVTGYSITSGYSWHWIRQFPGKGLEWIGSIHYSGGTNYNPSLKSRITISRDTSKNQFSLKLSSVTAADTAVYFCARMTTAPRYPFDYWGQGTLVTVSS

SEQ ID NO: 27 - protein, 120 aa, synthetic construct
DVQLQEWGAGLLKPSETLSLTCAVYGYSITSGYSWHWIRQPPGKGLEWIGSIHYSGGTNYNPSLKSRVTISRDTSKNQFSLKLSSVTAADTAVYYCARMTTAPRYPFDYWGQGTLVTVSS

SEQ ID NO: 28 - protein, 120 aa, synthetic construct
DVQLQEWGAGLLKPSETLSLTCAVTGYSITSGYSWHWIRQFPGNGLEWIGSIRYSGGTNYNPSLKSRITISRDTSKNQFSLKLGSVTAADTAVYFCARMTTAPRYPFDYWGQGTLVTVSS

SEQ ID NO: 29 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARMTTAPRYPFDYWGQGTLVTVSS

SEQ ID NO: 30 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARETTAPRYPFDYWGQGTLVTVSS

SEQ ID NO: 31 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARGTTAPRYPFDYWGQGTLVTVSS

SEQ ID NO: 32 - protein, 120 aa, synthetic construct
DVQLQEWGAGLLKPSETLSLTCAVTGYSITSGYSWHWIRQFPGNGLEWIGSIHYSGGTNYNPSLKSRITISRDTSKNQFSLRLSSVTAADTAVYFCARMTTAPRYPFDYWGQGTLVTVSS

SEQ ID NO: 33 - protein, 120 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSS

SEQ ID NO: 34 - protein, 120 aa, synthetic construct
QVQLQESGPGLVKPSQTLFLTCTVSGYSITSGYSWHWIRQHPGKGLEWIGSIRYSGGTDYNPSLKSLVTISVDTSKNQFSLKLSSVTAADTAVYYCARMTTAPWYPFDYWGQGTTVTVSS

SEQ ID NO: 35 - protein, 120 aa, synthetic construct
QVQLQESGPGLVKPSQTLSLTCTVSGYYITSGYSWHWIRQHPGKGLEWIGSIRYSGGTDYNPSLKSLATISADTSKNQFSLKLSSVTAADTAVYYCARMTTAPWYPFDYWGQGTAVTVSS

SEQ ID NO: 36 - protein, 120 aa, synthetic construct; identical to SEQ ID NO: 22

SEQ ID NO: 37 - protein, 120 aa, synthetic construct; identical to SEQ ID NO: 32

SEQ ID NO: 38 - protein, 9 aa, synthetic construct
GYSITSGYS

SEQ ID NO: 39 - protein, 9 aa, synthetic construct
GYYITSGYS

SEQ ID NO: 40 - protein, 9 aa, synthetic construct
  VARIANT 3: note = X can be S or Y
GYXITSGYS

SEQ ID NO: 41 - protein, 7 aa, synthetic construct
IHYSGGT

SEQ ID NO: 42 - protein, 7 aa, synthetic construct
IRYSGGT

SEQ ID NO: 43 - protein, 7 aa, synthetic construct
  VARIANT 2: note = X can be H or R
IXYSGGT

SEQ ID NO: 44 - protein, 13 aa, synthetic construct
ARMTTAPRYPFDY

SEQ ID NO: 45 - protein, 13 aa, synthetic construct
ARMTTAPWYPFDY

SEQ ID NO: 46 - protein, 13 aa, synthetic construct
ARETTAPRYPFDY

SEQ ID NO: 47 - protein, 13 aa, synthetic construct
ARGTTAPRYPFDY

SEQ ID NO: 48 - protein, 13 aa, synthetic construct
  VARIANT 3: note = X can be M, E or G
  VARIANT 8: note = X can be R or W
ARXTTAPXYPFDY

SEQ ID NO: 49 - protein, 107 aa, synthetic construct
DIVMTQSQKFMSTSVGDRVSVTCKASQIVGSNVAWYQQKPGQSPKPLIYSASYRYSGVPDRFTASGSGTDFTLTITNVQSEDLAEYFCQQYSSHPLTFGAGTKLELK

SEQ ID NO: 50 - protein, 107 aa, synthetic construct
DIVMTQSPSSLSASVGDLVTITCKASQIVGSNVAWYQMKPGKSPKPLIYSASYLYFGVPSRFSGSGSGTDFTLTISSLQPEDVAEYFCQQYSSHPLTFGPGTKVEIK

SEQ ID NO: 51 - protein, 107 aa, synthetic construct
DIVMTQSPSSLSASGGDRVTITCKASQIVGYNVAWYQQKPGKSPKPLIYSASYLYSDVPSRFSASGSGTDFTLTISSLQPEDVAEYFCQQYSSHPLTFGPGTKVEIK

SEQ ID NO: 52 - protein, 107 aa, synthetic construct
VIVMTQSPSSLSASVGDRVTITCKASQIVGPNVAWYQQKPGKSPKPLIYSASYGYSDVPSRFSGSGSGTDFTLTISSLQPEDVAEYFCQQYSTHPLTFGPGTKVEIK

SEQ ID NO: 53 - protein, 107 aa, synthetic construct
DIVMTQSPSSLSASVGDRVTITCKASQIVGSNVAWYQQKPGKSPKPLIYSASYGYSDVPSRFSGSGSGTDFTLTISSLRPEDVATYYCQQYSSHPLTFGPGTKVEIK

SEQ ID NO: 54 - protein, 107 aa, synthetic construct
DIVMTQSPSSLSASVGDRVTITCKASQIVGSNVAWYQQKPGKSPKPLIYSARYQYSGVPFRFSGSGSGTDFTLTISSLQPEDVATYYCQQYSSHPLTFGPGTKVEIK

SEQ ID NO: 55 - protein, 107 aa, synthetic construct; identical to SEQ ID NO: 54

SEQ ID NO: 56 - protein, 107 aa, synthetic construct; identical to SEQ ID NO: 54

SEQ ID NO: 57 - protein, 107 aa, synthetic construct
DIVMTQSPSSLSASVGDRVTITCKASQIVGSNVAWYQQMPGKSPEPLIYSASYLYSDVPSRFSGSGSGTDFTMTISSLQPEDVATYYCQQYSSHPLTFGPGTKVEIK

SEQ ID NO: 58 - protein, 107 aa, synthetic construct
DIQMTQSPSSVSASVGDRVTITCKASQIVGSNVAWYQQKPGKAPEPLIYSASYLYSGVPSRFSGSGSGTDFTLTISSLQPEDFATYYCQQYSSHPLTFGQGTKLEIK

SEQ ID NO: 59 - protein, 107 aa, synthetic construct
DIQMTQSPSSVSASVGDRVTFTCKASQIVGSNVAWYQQKPGKAPEPLIYSASYLYSSVPSRFSGSGSGTEFTMTISSLEPEDFATYYCQQYTTHPLTFGPGTKVEIK

SEQ ID NO: 60 - protein, 107 aa, synthetic construct
DIQMTQSPSSVSASVGDRVTITCKASQIVGSNVAWYQQKPGKAPMPLIYSASYGYSGVPSRFSGSGSGTDFTLTISSLQPEDFATYYCQQYSSHPLTFGQGTKLEIK

SEQ ID NO: 61 - protein, 107 aa, synthetic construct
DIVMTQSPSSLSASVGDLVTITCKASQIVGSNVAWYQMKLGKSPKPLIYSASYLYFGVPSRFSGSGSGTDFTLTISSLQPEDVAEYFCQQYFSHPLTFGPGTKVEIK

SEQ ID NO: 62 - protein, 107 aa, synthetic construct
DIVMTQSPSSLSASVGDRVTITCKASQIVGSNVAWYQQKPGKSPKPLIYSASYLYSDVPSRFSGSGSGTDFTMTISSLQPEDVATYYCQQYSSHPLTFGPGTKVEIK

SEQ ID NO: 63 - protein, 6 aa, synthetic construct
QIVGSN

SEQ ID NO: 64 - protein, 6 aa, synthetic construct
QIVGYN

SEQ ID NO: 65 - protein, 6 aa, synthetic construct
QIVGPN

SEQ ID NO: 66 - protein, 6 aa, synthetic construct
  VARIANT 5: note = X can be S, Y or P
QIVGXN

SEQ ID NO: 67-69 - skipped (3 entries)

SEQ ID NO: 70 - protein, 9 aa, synthetic construct
QQYSSHPLT

SEQ ID NO: 71 - protein, 9 aa, synthetic construct
QQYSTHPLT

SEQ ID NO: 72 - protein, 9 aa, synthetic construct
QQYTTHPLT

SEQ ID NO: 73 - protein, 9 aa, synthetic construct
QQYFSHPLT

SEQ ID NO: 74 - protein, 9 aa, synthetic construct
  VARIANT 4: note = X can be S, T or F
  VARIANT 5: note = X can be S or T
QQYXXHPLT

SEQ ID NO: 75 - protein, 450 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 214 aa, synthetic construct
DIQMTQSPSSVSASVGDRVTITCKASQIVGSNVAWYQQKPGKAPEPLIYSASYLYSGVPSRFSGSGSGTDFTLTISSLQPEDFATYYCQQYSSHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 18 aa, synthetic construct
CFGPNPNQCCHDECAGGC

SEQ ID NO: 78 - protein, 5 aa, synthetic construct
PNPNQ

SEQ ID NO: 79 - protein, 5 aa, synthetic construct
DECAG

SEQ ID NO: 80 - protein, 1210 aa, Homo sapiens [sequence omitted]

SEQ ID NO: 81 - protein, 405 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGLS

SEQ ID NO: 82 - protein, 705 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 83 - protein, 628 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGS

SEQ ID NO: 84 - protein, 1186 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 85 - protein, 381 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGLS

SEQ ID NO: 86 - protein, 681 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 87 - protein, 604 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGS

SEQ ID NO: 88 - protein, 621 aa, Homo sapiens [sequence omitted]

SEQ ID NO: 89 - protein, 23 aa, Homo sapiens
IATGMVGALLLLLVVALGIGLFM

SEQ ID NO: 90 - protein, 542 aa, Homo sapiens
RRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 91 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 92 - protein, 8 aa, synthetic construct
GFSLTNYG

SEQ ID NO: 93 - protein, 7 aa, synthetic construct
IWSGGNT

SEQ ID NO: 94 - protein, 13 aa, synthetic construct
ARALTYYDYEFAY

SEQ ID NO: 95 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 96 - protein, 6 aa, synthetic construct
QSIGTN

SEQ ID NO: 97 - skipped

SEQ ID NO: 98 - protein, 9 aa, synthetic construct
QQNNNWPTT

SEQ ID NO: 99 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC